(19) United States
(12) Patent Application Publication
(10) Pub. No.: US 2024/0024594 A1
(43) Pub. Date: Jan. 25, 2024

(54) NASAL APPLICATOR

(71) Applicant: HATCHMORE LABS GMBH, München (DE)

(72) Inventors: Klaus SINNER, Muenchen (DE); Francois Henri Mathieu Marie SOUREN, Muenchen (DE)

(21) Appl. No.: 18/040,972

(22) PCT Filed: Aug. 9, 2021
(86) PCT No.: PCT/EP2021/072189

§ 371 Date: Feb. 7, 2023

(30) Foreign Application Priority Data
Aug. 10, 2020 (DE) 10 2020 121 044.2
Dec. 30, 2020 (EP) 20217882.8

(51) Int. Cl. A61M 15/00

(52) U.S. Cl. CPC A61M 15/0083; A61M 15/008; A61M 15/009; A61M 15/0066; A61M 2210/0618

(57) ABSTRACT

The present invention relates to a nasal applicator (100) for administering nasally at least one medical substance (S), in particular an analgesic, having a housing (2), respectively comprising or connected to: a substance reservoir (R) for holding a quantity of the substance (S); a requesting device (1) to be actuated by the user with the aim of calling forward a next application dose (Dₙ) of the substance (S); a dispenser (3) for applying application doses (D₀, D₁, D₂, . . . , Dₙ₋₁, Dₙ, Dₙ₊₁, . . . ) upon actuation of the requesting device (1) at a respective delivery time (tA₀, tA₁, tA₂, . . . , tAₙ₋₁, tAₙ, tAₙ₊₁, . . . ); a connection site (4a) for a nasal attachment (4), or a nasal attachment (4) or a nasal piece; an application device (17) for applying an application dose of the substance (S) through the connection site (4a) or the nasal attachment (4) or the nasal piece and/or out of the nasal applicator (100).

The present invention relates to a drug dispensing device, in particular a nasal applicator, according to claim 1. It further relates to a system according to claim 36.

Nasal applicators by which a patient may nasally administer a drug or an agent to themselves are known from practice.

It is an object of the present invention to propose a further nasal applicator.

The object according to the present invention is achieved by the nasal applicator having the features of claim 1, and by the system having the features of claim 36.

According to the present invention, a drug dispensing device (for nasal, inhalative, intravenous, oral, buccal, sublingual, etc. dispensing), in particular a nasal applicator for administering or nasally administering at least one substance, e.g. a medical or non-medical substance, e.g. a medical or non-medical agent, is proposed. The medical substance may in particular be an analgesic. For this purpose, the drug dispensing device or the nasal applicator comprises a housing.

The drug dispensing device comprises, or is connected to, a substance reservoir for holding a quantity of the substance.

The drug dispensing device is connected to, or comprises, a requesting device to be actuated by the user/patient. The requesting device is programmed or differently prepared to allow the user of the drug dispensing device to request a next application dose of the substance by actuating the requesting device. Thus, the purpose or aim of the requesting device is to be able to release an application dose.

The drug dispensing device further comprises, or is connected to, a dispenser. The dispenser is provided for dispensing application doses in response to an actuation of the requesting device. The dispensing occurs, if so, at a respective so-called dispensing point of time.

The drug dispensing device further comprises a connection site for a nasal attachment or nasal piece, or is connected to a nasal attachment or nasal piece.

Finally, the drug dispensing device comprises, or is connected to, an application device for actively applying an application dose of the substance via the connection site or the nasal attachment or the nasal piece and/or out of the nasal applicator.

The system according to the present invention comprises one or several nasal applicators according to the present invention and one or several peripheral devices, wherein one or several of the nasal applicators are in signal communication or communication connection with one or several of the peripheral devices, in particular via the control device of the nasal applicator, or are prepared or programmed for this purpose.

When reference is made herein to a user, then this includes the patient who medicates themselves, the caregiver, the physician, patient relatives, and more, who, for example, at the behest of the patient, uses the drug dispensing device as described herein in medicating the patient.

In all of the above and following statements, the use of the expression “may be” or “may have” and so on, is to be understood synonymously with “preferably is” or “preferably has,” and so on, respectively, and is intended to explain an embodiment according to the present invention.

Whenever numerical words are mentioned herein, the person skilled in the art shall recognize or understand them as indications of a numerical lower limit. Unless it leads the person skilled in the art to an evident contradiction, the person skilled in the art shall comprehend the specification for example of “one” (also “a/an”) as encompassing “at least one”. This understanding is also equally encompassed by the present invention as the interpretation that a numeric word, for example, “one” (also “a/an”) may alternatively mean “exactly one”, wherever this is evidently technically possible for the person skilled in the art. Both understandings are encompassed by the present invention and apply herein to all used numerical words.

Whenever “programmed”, “provided”, or “configured” is referred to herein, it is also disclosed to interchange these terms.

Where reference is made herein to “programmed”, “provided” or “configured” to perform a step or action, it is also disclosed that this step or action is optionally performed automatically, e.g. by the control device.

Advantageous developments of the present invention are each subject-matter of the dependent claims and embodiments.

Whenever an embodiment is mentioned herein, it is then an exemplary embodiment according to the present invention.

When it is disclosed herein that the subject-matter according to the present invention comprises one or several features in a certain embodiment, it is also respectively disclosed herein that the subject-matter according to the present invention does, in other embodiments, likewise according to the present invention, explicitly not comprise this or these features, for example, in the sense of a disclaimer. Therefore, for every embodiment mentioned herein it applies that the converse embodiment, e.g. formulated as negation, is also disclosed.

Embodiments according to the present invention may comprise one or several of the features mentioned above or in the following in any combination being technically possible.

Whenever a suitability, a purpose, a step or a method step is mentioned herein, the present invention also encompasses the corresponding programming or configuring of an apparatus or of a section thereof, suitable for achieving or executing.

Although the present invention is not limited to a nasal applicator, but relates to drug dispensing devices as such, the following will focus on nasal applicators. Statements, explanations, and advantages made with respect to the nasal applicator apply undiminished to drug dispensing devices, and vice versa. In some embodiments, the terms “drug dispensing device” and “nasal applicator” may be synonymous. In several embodiments, these terms are interchangeable. Thus, what is set forth herein for the “drug dispensing device” also applies to the “nasal applicator”, and vice versa.

The applicator may optionally be usable for various forms of application, for example, sublingual, inhalative, and even intravenous. The connection site disclosed herein may be designed to be connected to a wide variety of connections through which a liquid may be conveyed, such as e.g. an impaction baffle or step baffle which breaks up the liquid into small droplets, a two-jet atomizer (impinging jet), a porous membrane, a perforated plate (Rayleigh breakup principle), an infusion tube, a vortex chamber, a nasal cannula, other nasal adapters to produce small droplets, etc. Said connection site may be connected thereto respectively.

In some embodiments, the nasal applicator further comprises a dose chamber. The dose chamber is used to temporarily receive the application dose of the substance held in the substance reservoir.

In several embodiments, the nasal applicator further comprises a loading device for loading the dose chamber with the application dose of the substance from the substance reservoir.

In some embodiments, the loading device is a pump.

In several embodiments, the loading device is a membrane that diffuses fluid into the dose chamber over time.

In other embodiments, the loading device is a dropper, alternatively a bellows, or sleeve, a bolus system, or similar.

In several embodiments, the concentration can be mixed by oneself to simulate different doses. In other words:

Different mixtures of carrier liquid and agent are provided in a dose chamber of constant and/or variable size (e.g. 100 μL) in order to achieve the desired concentration, e.g. 50 μg/100 μL or 60 μg/100 μL, etc.

For this purpose, and optionally additionally or alternatively for other purposes, a second fluid chamber may be provided in addition to the substance reservoir, which second fluid chamber comprises or is provided to receive a second fluid (preferably a liquid) different from the substance of the reservoir, such as a fluid for diluting the substance with the aim of adjusting a desired concentration.

A pump, or further pump, for applying the second fluid from the second fluid chamber may be provided, as may any other loading device such as, e.g. a syringe.

The second fluid chamber, or further, third fluid chamber, may comprise, or be provided for, a desiccant.

Instead of or in addition to the desiccant, an antidote for the, or an, agent of the substance may be provided, a neutralizing agent, to reduce or block the pharmacological effect.

In several embodiments, the application device is arranged to apply, in particular actively, the application dose of the substance present in the dose chamber via the connection site, the nasal attachment or the nasal piece, and/or out of the nasal applicator.

In some embodiments, in the case of a motor-driven direct piston drive, i.e. a system in which the vortex chamber is directly coupled to a syringe and/or cylindrical ampoule syringe (cartridge) that functions here as a reservoir and dose chamber—e.g. by a motor or otherwise—the required release speed, release force and/or pressure may be generated by applying pressure to the pressure plate of the syringe. The amount to be dispensed may be determined and controlled by a flow sensor. The flow sensor is provided to stop the movement of the application device in these embodiments, for example to stop the optional motor when the desired amount to be dispensed has been reached.

In some embodiments, an audio signal may optionally be emitted while and/or as long as a fluid is being delivered. Alternatively or additionally, in several embodiments, an audio signal may be provided after a completed application. Both various audio signals as well as various tonalities and tone pitches are encompassed by the present invention.

In some embodiments, the dose setting and/or the release is manual, or a combination of manual and automatic.

In some embodiments, the application device and/or the loading device comprises an energy storage for mechanical or electrical energy or spring energy.

In certain embodiments, the application device and/or the loading device comprises an energy storage for hydraulic and/or pneumatic energy.

In several embodiments, the stored energy is energy generated by the human body or by gravity. In these embodiments, the energy is stored by changing the density or by converting this energy into another form of energy.

In some embodiments, the application device and/or the loading device comprises a motor, preferably an electric motor.

In several embodiments, the motor may also be driven by compressed air (pneumatic, hydraulic) or by mechanical energy, for example spring energy and/or human applied energy. The motor may also be a pump.

In some embodiments, the application device and/or the loading device includes a spindle.

The spindle may be driven electrically, pneumatically, hydraulically, mechanically, or by spring energy.

The spindle may be designed in different ways, e.g. as a linear system (linear axis) with, toothed belt and spindle drive for a linear movement, as ball bushings, as threaded spindle (ball screw or trapezoidal screw gear), as shaft guide, as grooved shafts, as cylindrical, flanged, rotary, short-stroke, long-stroke torque ball bushings for a torque transmission, as torque shafts with and without groove, as rail guide, as electric, pneumatic, or hydraulic cylinders, as telescopic cylinders (e.g. multi-stage hydraulic piston) or as single or double acting cylinder.

In some embodiments, the spindle may be smooth and driven e.g. by two pairs of ball bearings mounted at an angle and opposite to each other. A rolling ring gear (Uhing gear) may be provided.

In certain embodiments, the nasal applicator does not have a spring element nor a storage device for mechanical energy or spring energy.

In certain embodiments, the loading device and/or the application device does not have a spring element and/or no storage device for mechanical energy or spring energy.

In several embodiments, the motor is connected to the spindle.

In these embodiments, the motor is directly connected to the spindle. Alternatively, the motor and spindle are indirectly connected, i.e. a step-up or step-down gearing may be interposed.

Motor and spindle may be collectively referred to as gear (unit). In some embodiments, it may be a fixed gear, i.e. speed ratio and torque conversion are not variable. In other embodiments, the gear may be a variable gear (stepped and/or continuously variable). In further embodiments, the gear may be a gear for speed stepping and/or torque stepping and/or for reversing the direction of rotation.

Within the gear unit, the step-up or step-down gearing may be form-fit or force-fit, such as e.g. in a toothed (wheel) gear and/or a friction (wheel) gear and/or epicyclic gear (all three (wheel) gearings), a chain gear and/or belt gear (both traction gears, a planetary gear, a pressure gear (e.g. as in the hydraulic gear of a car brake), a linkage gear (e.g. as in the crankshaft gear of an internal combustion engine—crankshaft and connecting rod), a cam gear (e.g. as in the cam gear of a valve control), a screw and worm gear (e.g. as in a vise or jack), a linear chain gear, a friction gear, a power division gear (e.g. a differential gear), a multi-disc chain gear, a rolling ring gear, a coupling gear (e.g. crank gear, Maltese cross gear (Geneva drive)), or a gear with an elastic gear element (e.g. strain wave gearing).

In some embodiments, the nasal applicator further comprises a mechanism for changing the capacity volume of the dose chamber for the application dose of the substance. The changing of the capacity volume may be effected manually or automatically.

Such changing of the capacity volume may be done by changing the stroke path of the loading device or application device. Examples of that are: lengthening/shortening the travel path of the loading device or application device, twisting an inclined plane, by a curve, e.g. a cam, groove, heart curve, etc., an adjustable (end) stop, adjustable pins as travel limiters, a change in the dose chamber diameter, a change in the geometry of the loading device or of the application device (e.g. from round to oval), respectively, a change in the geometry of the dose chamber (e.g., from round to oval), and/or telescopic pistons extending outward or inward.

In some embodiments, it may be provided to change the capacity volume of the dose chamber by providing, by initially moving the piston (such as counterclockwise) by rotating the spindle in a first direction, an accordingly enlarging of the dose chamber in order to draw substance from the reservoir. To apply the application dose thus introduced into the dose chamber, the spindle is then rotated in the opposite direction.

In certain embodiments, it may be provided to effect filling and emptying the dose chamber by a crankshaft gear.

The crankshaft gear may comprise or consist of an eccentric shaft and a thrust rod arranged on or at the eccentric shaft being rotatable relative thereto.

The crankshaft gear serves to convert a rotary motion into an oscillating thrust motion, or vice versa. With reference to the design of the loading device or application device relevant here, this may result in a piston engine with a piston that moves back and forth in a straight line in the cylinder.

The control device may be programmed to both fill the dose chamber by rotating the motor drive in a first direction, and to empty it again by further rotating the motor drive in the same direction, with the aim of applying the next application dose. The quantity of the dose delivered in this case may be determined by rotating the motor drive by an appropriate angular value.

Alternatively, the control device may be programmed to fill the dose chamber by rotating the motor drive in a first direction, and to empty it again by rotating the motor drive in the second direction, opposite to the first, with the aim of applying the next application dose. The amount of the delivered dose may again be determined by rotating the motor drive by a corresponding angle value, which makes the adjustment of the dose variable. The crankshaft gear may be used advantageously in this regard.

In several embodiments, reducing the size of the dose chamber is encompassed by the present invention in which not the stroke path of the applying mechanism is changed, but rather the connecting side is moved.

If the dose chamber accommodates e.g. a maximum volume of 150 μL, then the following method may be appropriate, for which the control device may also be programmed: The dose chamber is filled with 150 μL with or by the suction stroke, i.e. it is completely filled. The application dose to be dispensed, however, is e.g. only 50 μL which is dispensed by reversing the path covered e.g. by the piston, which constitutes the applying stroke. The residual amount remaining in the dose chamber is thus 100 μL in this example. If the next application dose is e.g. 75 μL, the device does not have to perform a suction stroke, but instead performs the applying stroke immediately. The yet remaining amount still present in the dose chamber after this last application is 25 μL. If the subsequent application dose is e.g. 100 μL, this again requires a suction stroke of 75 μL. However, the dose chamber may alternatively be refilled to 150 μL.

Further alternatively, it is encompassed by the present invention that a dose chamber having a constant size (e.g., 150 μL) is always filled with only the desired volume to be applied (e.g., using a micropump, for example with 100 μL. In these embodiments, the application device is moved towards the connection site to first compress air. At a predetermined pressure, the valve opens and the air is forced through the vortex chamber together with the liquid. Alternatively, the compressed air is discharged through an aeration valve.

In some embodiments, the mechanism for changing the capacity volume of the dose chamber for the application dose of the substance is part of the dispenser.

When reference is made herein to a motor, the motor may be designed as a Wankel engine.

In several embodiments, the nasal applicator further comprises an electronic control device.

In some embodiments, the electronic control device is programmed to cause the application device to move at a plurality of speeds, forces, pressures, accelerations, and/or flow rates and/or to apply substance at a plurality of speeds, forces, pressures, accelerations, and/or flow rates. Thus, it may for example be provided to apply at a first, in particular pre-determined, speed, to apply at a second, in particular pre-determined, speed which is different from the first speed, and optionally at still further speeds. These applying speeds may be determined dependent on the amount to be delivered and/or the volume of substance to be delivered and/or the (respective) nature thereof.

In some embodiments, the nasal applicator comprises a second fluid chamber comprising a desiccant or an agent for neutralizing the substance.

In some embodiments, the application device and the loading device comprise, or consist of, identical components.

In several embodiments, the electronic control device is programmed to act on the mechanism for changing the capacity volume of the dose chamber.

In some embodiments, the loading device comprises at least one first one-way valve or check valve for loading the application dose of the substance into the dose chamber and/or the dose chamber is limited by at least one first one-way valve or check valve.

In several embodiments, the loading device optionally comprises at least one valve that opens at a predetermined pressure value (e.g. 4 bar (4000 hPa)). In other words, the dose chamber is pressurized by the application device, as soon as the application device moves, the pressure in the dose chamber increases to the predetermined pressure value (e.g. 4 bar (4000 hPa)), opens the valve and the valve remains open until the dose chamber is empty. Thus, the one-way valve may also display the function of a pressure switching valve.

Alternatively or additionally, it may be provided to open or close a valve by a pressure switching sensor or pressure sensor. This solution covers both the function of a check valve as well as the function of a pressure switching valve (alternatively: pressure sequence valve or switching valve) of another valve, a throttle, a barrier for volume flow, etc.

In some embodiments, the valve is mechanically switchable, in others electrically or electromagnetically, and in still other embodiments pneumatically or hydraulically.

A mechanically switchable valve is, e.g., a check valve with a spring element. Other embodiments of a mechanically switched valve are, e.g., disc check valve or ball check valve, swing check valve, or back pressure valve.

In several embodiments, the valve is a switchable three-way valve. This may also be mechanically, electrically or electromagnetically, pneumatically or hydraulically switchable.

In some embodiments, the one-way valve is integrated or partially integrated into the application device or loading device.

In several embodiments, the one-way valve is replaced by blocking the plug in the substance reservoir after the dose chamber is filled.

The one-way valve may be integrated into the vortex chamber, e.g., beyond the connection site, or in the nasal attachment, etc.

The vortex chamber may be shaped to perform the function of a valve. The vortex chamber may have such a valve, or may be limited to one side or defined in its extent by such a valve.

In some embodiments, for loading the dose chamber with the application dose of the substance, the loading device comprises at least one second valve, e.g., any valve as discussed herein or other valve, in particular a one-way valve or check valve. Alternatively or additionally, the dose chamber is delimited by at least one such valve, for example, a second one-way valve or check valve.

The second check valve, thereby, preferably comprises an opposite opening direction with respect to the dose chamber than the first check valve.

In certain embodiments, the function of the first valve and the function of the second valve may be provided together in a single component.

In several embodiments, the nasal applicator further comprises a locking device for temporarily locking the dispenser during a locking period that rebegins at or after the respective dispensing point of time of the dispensed application dose. The locking period has a locking period begin and a locking period end.

In some embodiments, the nasal applicator further comprises a dose-detection device for detecting the amount of the application dose dispensed by the dispenser at each dispensing point of time.

In several embodiments, the nasal applicator further comprises a data storage for storing at least the dispensing points of time of one or more previously dispensed application doses. Alternatively or additionally, the amounts of these application doses are also stored.

In certain embodiments, the dose chamber comprises or consists of a chamber for receiving the substance or the dosed amount of the substance. In particular, in these embodiments, the dose chamber is of variable size or variable capacity volume.

In other embodiments, the dose chamber is of non-variable size and/or of always the same capacity volume.

In certain embodiments, the dispenser is configured and arranged such that to dispense the amount of substance to be dispensed as a spray and/or atomized.

In certain embodiments, the nasal applicator does not comprise an energy storage and/or means for applying a predetermined amount of energy, particularly mechanical energy, to the energy storage.

In certain embodiments, the nasal applicator does not comprise means for releasing the predetermined amount of energy from the energy storage to the dose chamber in order to thereby subject the fluid therein to a predetermined increase in pressure from a low pressure to a higher pressure and initiate a dispensing of the fluid from the dose chamber.

In some embodiments, the drug dispensing device may further be connected to, or comprise, a locking device.

In several embodiments, the locking device is configured to temporarily lock the dispenser during a, in particular, predefined locking period. The locking period begins, for example after the dispensing of one, some or each application dose. It may thus be provided that a locking period begins again at or after the respective dispensing point of time of the dispensed application dose. The locking period is defined by its respective locking period begin and its respective locking period end. Locking periods set by the locking device may be of different durations or each may have the same duration.

Additionally, in several embodiments, the drug dispensing device comprises, or is connected to a dose-detection device. The dose-detection device is programmed to detect the amount (e.g. as a quantity, in units of weight, in units of volume, in units of substance such as moles, etc.) of the application dose respectively dispensed by the dispenser at each respective dispensing point of time. It may also be programmed to detect the amount of an application dose referred to herein as the initial dose.

Further, in several embodiments, the drug dispensing device is connected to, or comprises, an electronic control device.

The drug dispensing device may comprise, or be connected to, a data storage for storage. It may store and/or have at least the dispensing points of time of one or more application doses that have already been dispensed, which are thus, for example, before the next dispensing point of time or in the past, and/or the amounts of these application doses.

The drug dispensing device may comprise, or be connected to, a detection device programmed to detect the actuation of the requesting device by the user. The detection device may detect at least one actuation of the requesting device by the user that has occurred or is occurring at an actuation point of time as an actuation behavior and is preferably configured, programmed and/or provided for this purpose accordingly. The actuation behavior aims at releasing, dispensing or having dispensed a next or future application dose and/or checking the circumstances, for example whether a previous dispensing point of time has already been before a sufficiently long time period in the past. The actuation behavior may be the actuation of the requesting device or may be detected therefrom by evaluation.

The electronic control device may be programmed to read data stored in the data storage. It is further programmed to evaluate the actuation behavior of the user detected by the detection device.

The result of the evaluation of the user's actuation behavior by the electronic control device may, in some embodiments, influence the predetermination of the amount of the next or future application dose.

The electronic control device may be programmed to determine the time that has elapsed since the dispensing point of time of one or more already dispensed application doses until the actuation of the requesting device and to take this into account, particularly quantitatively, in the predetermination.

The electronic control device may be programmed to determine and take into account, particularly quantitatively, the time that has elapsed since the dispensing point of time of one or more of the already dispensed application doses until the actuation of the requesting device, in predetermining, at a predetermination point of time, the amount of the next or further application dose that is dispensable to the user at a next dispensing point of time in the event that the user actuates the requesting device outside of a locking period that may still be ongoing when the requesting device is actuated.

In some embodiments, the nasal applicator and/or one or more devices thereof are programmed to predetermine the amount of the next or further application dose already at a predetermination point of time that lies within a locking period.

In several embodiments, the nasal applicator and/or its devices are not programmed to be able to enter a time period or point of time on the part of the user after which/at which the effect or a predetermined effect of the administered amount of agent is to be terminated, broken down or fallen below.

In some embodiments, the nasal applicator and/or its devices are not programmed to compensate for or change the predetermined amount of the next or further application dose using a stored and/or retrieved compensation factor for the amount.

In several embodiments, the nasal applicator is not designed or configured to evaporate or vaporize an application dose.

In several embodiments, the substance in the reservoir is permanently pressurized, such as by a spring arranged in order to act compressively on the internal volume of the reservoir.

The dose chamber may be designed to be, when being equipped with the next or further application dose to be dispensed, brought into the volume required in order to receive this dose by a corresponding movement by, for example, the motor, the spindle and/or the loading device. It may subsequently be reduced, for applying the dose.

In some embodiments, the dose chamber has a variable size or a variable capacity volume. This is preferably automatically adjusted before or for the purpose of equipping it with the next or further dose by motor, spindle and/or loading device.

In several embodiments, the control device is programmed to first increase the volume of the dose chamber for the filling thereof and then decrease it again for the emptying thereof, using motor, spindle and/or loading device. For this purpose, motor and/or spindle preferably rotate both in a first direction and later in a second direction opposite to the first.

In some embodiments, the dose chamber is delimited by inner wall sections of a piston, a movable plunger, and/or by two valves. In several embodiments, the dose chamber is formed thereby.

In several embodiments, one or several of the valves delimiting the dose chamber are check valves or other mechanically actuated valves.

In some embodiments, the valves delimiting the dose chamber are stationary, which may increase their precision. Stationary here means that they are not movable with, for example, the movement of the piston.

In certain embodiments, the valves delimiting the dose chamber are arranged at or associated with a common end of the dose chamber, e.g., are both arranged in an upper area, or both associated with the nasal attachment.

In several embodiments, the control device is programmed to move the loading device both in a first direction and subsequently in a second direction opposite to the first direction after actuation of the requesting device with the aim to administer the next or further application dose.

In some embodiments, the nasal applicator does not comprise a piston pump.

In several embodiments, the nasal applicator does not comprise a device for delimiting the maximum possible stroke path of a piston pump.

In some embodiments, the nasal applicator does not comprise an electrical valve-actuation device.

Further, it may be programmed in order to, for example during the use of the nasal applicator and/or preferably without the intervention of a medical staff member, the manufacturer or other persons, predetermine or to determine, for instance select, calculate or define, at a predetermination point of time (or determination point of time), preferably out of a plurality of possible amounts, the amount of the next application dose—which is optionally not known prior to the beginning of the treatment and variable, e.g., at least within prespecified thresholds—e.g. from a plurality of possible or definable application doses wherein the application dose is dispensable to the user as the next application dose, e.g. at a next dispensing point of time, or it is dispensable for a delivery to said user in the case that said user actuates the requesting device outside a locking period, if such a locking period is set. The amount of the next application dose may be unknown in advance. The amount of the next application dose may not be stored in advance, i.e., may not be taken from a storage; rather, it must be determined or predetermined. In some embodiments, the amount of the next application dose is unknown until the control device has predetermined or determined it, such as by calculating it. Thus, it may be unknown until the predetermination point of time. It may be variable.

The electronic control device may be programmed to predetermine the amount of the next application dose only after a previous actuation of the requesting device.

The electronic control device may be programmed not to predetermine the amount of the next application dose until at least the first actuation of the requesting device.

The electronic control device may be programmed to determine the amount of the respective next application dose for each application dose, or at least for several application doses.

The electronic control device may be programmed to store the amounts of dispensed application doses detected by the dose-detection device in the data storage.

Such a predetermination point of time lies at or after the actuation point of time. The predetermination step is performed taking into account data read out from the data storage. The data read out here comprises preferably at least the dispensing point of time of one or more of the application doses already dispensed on the one hand, or, on the other hand, the time elapsed between the dispensing point(s) of time of the application doses already dispensed and the dispensing point of time lying at or after the next actuation point of time. Alternatively or additionally, the read data preferably also comprises the amount of one or several of the application doses already dispensed, in particular by the drug dispensing device.

In several embodiments, the amount of the next application dose is different from the amount of at least one, several, or of all previously predetermined prior application doses.

In certain embodiments, the read data does not encompass data input by the patient with respect to the patient's condition, or is not based exclusively thereon, rather at most, also. Such data may be patient-specific control data and/or data representing (health) complaint duration, (health) complaint severity, and/or (health) complaint frequency.

In several embodiments, the dispenser is not the dose-detection device. While the dispenser is dispensing the application dose to the patient, the dose-detection device detects the amount of what was actually dispensed.

In some embodiments, the dose-detection device does not determine the amount of the next application dose.

In some embodiments, the nasal applicator does not comprise a display that would be controlled to indicate a remaining number of application doses and/or a number of application doses that have already been dispensed.

In some embodiments, the nasal applicator does not have a plurality of vials, particularly not identical vials.

In several embodiments, no reminder signal, e.g. directed to the user, to actuate the requesting device again, is issued after a predetermined maximum time period has elapsed.

In some embodiments, the data storage further comprises pharmacological, pharmacodynamic, pharmacometric and/or pharmacokinetic data, in particular concerning the substance that is, or is to be, applied. These data may be or comprise models, in particular PK models, or PK curves. This data may be formulas and/or may be or may have been generated based thereon, e.g. on-line, by the electronic control device and/or in real time. They may have been detected or compiled on a patient-specific basis. Alternatively or additionally, other data associated with the substance may be encompassed by, or stored, in the data storage.

The data may comprise, for example the dose half-life and/or the elimination half-life or terminal half-life of the substance.

The dose half-life is the time required for the concentration (e.g., in plasma, blood, site of action (CNS, central nervous system), adipose tissue, liver, etc.) to decrease to the half (50%) of the administered dose.

The elimination or terminal half-life is the time required for the concentration prevailing at pseudo-equilibrium to decrease to half the value (50%).

The electronic control device is optionally programmed to additionally read out such data from the data storage.

Furthermore, the predetermination of the next or further application dose of the substance is carried out with additional consideration of the additionally read-out data. Thereby, the predetermination point of time, and optionally also the actuation point of time, may correspond to the dispensing point of time of the next application dose, but it may also be before the dispensing point of time.

The locking period end of the locking period may correspond to, or be derived from, the point of time of the maximum concentration (this point of time is also referred to herein as: tₘₐₓ) between the dispensing of two successive application doses of the substance.

In certain embodiments, the locking period may depend on the concentration maximum and may accordingly be or become set by the physician or other authorized persons or automatically by the drug dispensing device, alternatively on a defined concentration, alternatively on the time until this defined concentration is fallen below.

In several embodiments, the electronic control device is further programmed to prompt a dispensing of the next or further application dose at the predetermined amount at the corresponding dispensing point of time using the dispenser. The electronic control device is further programmed to subsequently store or cause to be stored in the data storage the amount of an application dose and/or a concentration resulting therefrom, for example as an amount indication or volume indication etc. of the substance, of the dispensed next application dose. Further, the control device is preferably programmed to prompt, e.g. simultaneously with or subsequent to the delivery of the application dose, the locking device to temporarily block the dispenser for a specific locking period.

The locking period begins e.g. from or after the dispensing point of time of this next application dose and is defined by a locking period begin and/or a locking period end, which may also apply to any other locking periods mentioned herein.

The dispensing point of time of the dispensed next application dose may be stored in the data storage, and the control device may be programmed for this purpose.

In some embodiments, the electronic control device is further programmed to execute, or prompt or initiate, the sequence of the above-mentioned further steps also in connection with one or several of the dispensing of further application doses subsequent to the next application dose, which are subsequent in time to the next application dose.

In several embodiments, the electronic control device is programmed to read out data from the data storage, which data encompasses, e.g., PK curves and/or PK models, and which represent a time course of the concentration of the substance in the body, in particular in the blood, of the patient. Based on these curves or models, the electronic control device may calculate a next maximum of the concentration. The next maximum may be the maximum that the concentration takes between immediately successive dispensing points of time. It may be the result of cumulative application doses.

In certain embodiments, this maximum of concentration is calculated and taken for the definition of the locking period. When defining the locking period, the point of time at which this maximum is reached may be taken into account.

In some embodiments, the electronic control device is further programmed to determine the next application dose such that the next maximum of the concentration will not exceed a predetermined maximum value or a predetermined maximum. In these embodiments, the determination of the application dose is carried out based on data stored in the data storage, particularly PK curves or models, and a therapeutic maximum maintained in the data storage. The therapeutic maximum may be the maximum of a single application dose or the maximum of a sum of several applications or the maximum of a sum of several applications over a certain time or within a certain time interval.

In several embodiments, evaluating the detected actuation behavior of the user encompasses determining the actuation point of time at which the user wishes to request the next or further application dose. Alternatively, evaluating the user behavior encompasses determining the time period that lies between this actuation point of time and the locking period end of the last expired locking period.

In this, predetermining encompasses considering the determined next actuation point of time or considering the determined time period.

In some embodiments, the medication application device or nasal applicator further comprises a feedback device. It serves the user to provide feedback, for example, with respect to their well-being, pain condition, or similar. The feedback device, which may be a switch, a touch surface, etc., may e.g. be provided in order to be actuated by the user when, or as long as, they are pain-free or otherwise able to make a statement about the effect of the substance in their body. Thus, it may be agreed that the user actuates the feedback device for the first time, e.g. presses it, when the pain is perceived as tolerable for the first time after the application dose last applied to the user. It may further be agreed that the user no longer actuates the feedback device from the point of time when they first feel pain again (due to decreasing concentration of the substance in the body).

The feedback device may be identical to the requesting device, e.g. with its own switch evaluation, for example by being longer, shorter, more frequent, or otherwise differently, actuated than the requesting device, alternatively, it may be a separate device for actuation by the user.

Here, the detection device is programmed to detect a completed actuation of the feedback device by the user at least at one, preferably first, feedback point of time.

Further, the control device is programmed herein for determining the concentration of the substance present in the user's body or blood at least at the one feedback point of time.

In this, determining can always be calculating, reading out and/or reading (off).

In addition, the control device is programmed to set a target concentration or its maximum value to a value respectively below, above or equal to the determined concentration.

Whenever reference is made herein to a “target concentration,” this may be the concentration that produces a desired therapeutic effect.

In several embodiments, the detection device is further programmed to detect at least one actuation of the requesting device by the user at different actuation points of time, particularly after the locking period. Alternatively or additionally, the detection device is programmed to detect a, feedback point of time, preferably a last feedback point of time, from which further actuations of the feedback device by the user are absent at least until the dispensing point of time of the next application dose. Optionally, the present invention also encompasses the storage of these feedback points of time.

The control device may be further programmed for determining concentrations of the substance in the body, in particular in the blood, for example on the basis of the read-out data, such as, e.g., PK curves, PK models, etc. In this, for example, both the concentration at the, preferably first, feedback point of time and the concentration at the, preferably first, actuation point of time (e.g. since the end of the last locking period) or at the last feedback point of time are relevant. These concentrations may be or become associated with the respective points of time.

Furthermore, the control device may be further programmed for determining or selecting a temporal concentration course from a group of temporal concentration courses. In determining the temporal concentration course, both the concentration at the, preferably first, feedback point of time and the concentration at the, preferably first, actuation point of time or the last feedback point of time are considered.

When reference is made herein to a “group of temporal concentration courses,” this means a group of concentration courses that relate to or are derived from the same dose (e.g., 50 μg of fentanyl). Specifically, concentration courses encompass those that reflect the temporal course of concentration for the mean of all patients in a collective studied based on that dose, or the mean plus/minus one or several standard deviations. A variety of such courses or data may be stored in the data storage. From it, the group of temporal courses under consideration may be selected.

Further, the control device may be programmed for taking into account the temporal individual concentration course thus determined upon predetermining the amount of one or several of the subsequent application doses, in particular for taking into account the determined individual PK curve.

In some embodiments of the nasal applicator, the control device is further programmed for taking into account the determined target concentration or its maximum value upon predetermining the amount of the next application dose. Alternatively or additionally, it may be provided that the determined temporal individual concentration course, in particular the determined individual PK curve, is taken into account during predetermination.

In several embodiments, the control device is further programmed, after delivery of a starting dose or an initial dose at an earlier point of time, at a respective first (or herein referred to as “initial”) point of time of actuation of the requesting device by the user (preferably after expiration of a locking period following the dispensing of the initial dose, in which no application dose is applied to the user upon actuation of the requesting device), for determining a concentration associated with this actuation point of time, namely for each of the concentration courses (stored in the data storage) of the/a group, or to derive it therefrom. This first actuation point of time is temporally after the dispensing point of time of the initial dose. The control device is further programmed for defining the concentrations thus determined as respective estimated target concentration of the associated temporal concentration course. If, in simplified terms, there has been an initial dose, and if the user has wanted to request a further application dose after the expiry of a locking period following this, a concentration is assigned to the time at which the user attempted to request the further application dose for each of the temporal concentration courses considered. Since the concentration thus assigned was determined by calculation and is the result of a hypothesis that still requires verification and confirmation, the concentration thus determined is referred to herein as the estimated target concentration. If the group of considered temporal courses has three elements (courses), three estimated target concentration values are obtained. One makes, in other words, three hypotheses. They state how high the concentration in the body should be, if the first, the second or the third concentration course from the group would be true for the considered user.

An initial dose may also be referred to herein as a first or preceding application dose dispensed at a dispensing point of time, which may accordingly be the first dispensing point of time. The initial dose may be the actual very first application by the drug dispensing device.

The control device may further be programmed for applying the first application dose following the initial dose in response to actuation of the requesting device at the first actuation point of time.

Furthermore, the control device may be programmed for determining a point of time at which, after application of this first application dose, the concentration in the body, in particular in the blood, will or should have dropped again to the estimated target concentration of the associated temporal concentration course. This point of time is also referred to herein as the calculated target concentration point of time. In order to respectively calculate it (i.e. for each concentration course from the group), the same temporal concentration courses of the group are used again, respectively, for which a calculated target concentration point of time has already been respectively determined. It should be noted that differences between the amount of the initial dose and the subsequent, first application dose are taken into account here. For example, if the initial dose was 50 μg of fentanyl, then the three selected temporal concentration courses of the group have indicated how these 50 μg of fentanyl might be proven in the body. A first of the three concentration courses might, for example, assume that the course will behave or run as observed on average in a collective. A second might, for example, assume that the course will behave as the first course did by plus one standard deviation, plus 10%, and so on. A third may, for example, assume that the course will behave like the first course by minus one standard deviation, minus 10%, and so on. However, if the subsequent, first application dose is now for example not also 50 μg, but only 30 μg of fentanyl, the same three selected temporal concentration courses of the group are respectively assumed. They are only no longer calculated on 50 μg, but on 30 μg fentanyl.

The control device may further be programmed for directly or indirectly detecting or determining the second actuation point of time for requesting a second application dose or an application dose following the subsequent, first application dose itself, e.g. second, application dose.

In addition, the control device may be programmed for determining a temporal concentration course from the group as an individual temporal concentration course, in particular as an individual PK curve. In this, the respective difference between the calculated target concentration point of time of each temporal concentration course from the group and the second actuation point of time is taken into account. In particular, the temporal concentration course which has the smallest difference between its calculated target concentration point of time and the second actuation point of time is considered to be the individual temporal concentration course.

The control device may further be programmed for taking into account the determined temporal individual concentration course upon predetermining the amount of one or several of the subsequent application doses, wherein the temporal individual concentration course may be in particular an individual PK curve or an individual PK model.

In some embodiments, the control device is further programmed for determining the concentration of the substance present in the user's body or blood. The determining is carried out at least at one considered actuation point of time or at least at one considered predetermination point of time. Further, the control device is programmed for setting this concentration as the target concentration.

Here, the control device is further programmed for setting, at the next predetermination point of time, the amount of the/a next application dose in such a way that the concentration in the user's body or blood will only drop to the target concentration again after a predetermined time period has elapsed. This time period may be adjustable, for example by the attending physician, and begins with the dispensing point of time of the next application dose.

For this purpose, analogously as described above, the invariable temporal individual concentration course, i.e. the PK curve of the user, may be taken as a basis and/or taken into account.

In several embodiments, the electronic control device is further programmed for taking into account data read from the data storage in the step of predetermining the amount of the next application dose. This data encompasses both the dispensing point of time of one or more of the already dispensed application doses, on the one hand, and the amount of one or several of the already dispensed application doses, on the other hand. It is encompassed by the present invention that, in this regard, as an alternative to the dispensing point of time, the time elapsed between the dispensing point(s) of time of the already dispensed application doses and the dispensing point of time after the next actuation point of time may be considered.

In some embodiments, evaluating the user's actuation behavior detected by the detection device encompasses detecting actuations of the requesting device by the user at actuation points of time within a locking period. This may be done in any arbitrary, a particular, i.e., the first and/or the second, etc., or in any locking period. In certain embodiments, storing this actuation behavior in or on a device suitable for this purpose, e.g. the data storage mentioned herein, is also optionally provided.

The control device is further programmed for determining a concentration of the substance in the body, in particular in the blood, which is or may be associated with the respective actuation points of time within one of the locking periods.

The control device is further programmed for setting a target concentration or its minimum value to a value above, below or equal to the associated concentration, respectively.

In certain embodiments, the procedure mentioned supra may be repeated multiple times, i.e. looped. The target concentration thus approximated, or the minimum value thus approximated, may optionally be reset to zero (0) after one or after each locking period.

In several embodiments, the detection device of the nasal applicator according to the present invention is further programmed for detecting and optionally storing at least one actuation of the requesting device by the user at least at one actuation point of time after the locking period.

In this, the control device is further programmed for determining a concentration in the body, in particular in the blood, which is associated with the actuation point of time after the locking period, respectively.

Further, the control device is programmed for determining a temporal concentration course of the group. In this, both at least one, preferably the last, actuation point of time within the (arbitrary) locking period and at least one, preferably the first, actuation point of time after the locking period are taken into account.

The control device is further programmed for taking into account the determined temporal individual concentration course, in particular the determined individual PK curve, upon predetermining the amount of one or several of the subsequent application doses.

In some embodiments of the nasal applicator, the control device is further programmed for taking into account the determined target concentration or its minimum value and/or the determined temporal individual concentration course, in particular the determined individual PK curve, upon predetermining the amount of the next application dose.

In several embodiments, evaluating the user's actuation behavior detected by the detection device encompasses evaluating actuations of the requesting device by the user at actuation points of time within one of the locking periods. This may be done in an arbitrary locking period, a particular one, i.e. the first and/or the second, etc., or in any of the locking periods. In certain embodiments, storing this actuation behavior in or on a device suitable for this purpose is also optionally provided.

Further, the control device may be programmed for determining an increase in the time intervals between successive actuation points of time within one of the (considered) locking periods, alternatively the absence of further actuation points of time within the considered locking period. In addition, the control device may be programmed for determining a point of time from which the degree of increase or the absence satisfies predetermined criteria.

For example, it may be determined as criteria that as soon as, e.g., 50%-75% of the locking period T_vainₙ has elapsed, it may be assumed that the actuation points of time are substantially further apart in a predetermined manner than before, and/or if no actuation has occurred for a, e.g. predetermined, time period, it is assumed that the reaction to the therapeutic effect has become noticeable/perceptible and/or the target concentration has been reached.

For some embodiments, it may be defined that if computationally, e.g. 80% or other predetermined portion of the calculated maximum concentration has been reached and there are still unsuccessful requesting attempts, i.e. the desired therapeutic effect has not yet been achieved, then the end of the current locking period is advanced because the remaining, e.g. 20% of the concentration will not be sufficient to bring the user to the target concentration (C_{ED}) under the currently applied regimen.

For some embodiments, it may be defined that if computationally, e.g. 80% of the calculated maximum concentration has been reached and there are still unsuccessful requesting attempts, i.e. the desired therapeutic effect has not yet been achieved, then the next predetermination point of time is advanced knowing that there will likely be a requesting attempt by the user at the end of the locking period.

If these time intervals become larger, this means that the desired therapeutic effect will begin to take effect, perhaps slowly, but surely. Thus, in the event that an actuation by the user occurs after the end of the locking period, the dose may be smaller or calculated as if there were no therapeutic effect.

Further, the control device may be programmed for determining a concentration of the substance in the body, particularly in the blood, associated with the point of time.

The control device may be further programmed for setting the target concentration or its minimum value to a value respectively above, below, or equal to the associated concentration.

In some embodiments, the electronic control device is further programmed to evaluate at least one actuation of the requesting device by the user at least at one actuation point of time, which is after the locking period.

Optionally, storing the actuation in or on a device suitable for this purpose is also encompassed by the present invention.

In this, the control device is further programmed for determining a concentration in the body, in particular in the blood, for the actuation point(s) of time after the locking period.

In this regard, the control device may be further programmed for determining a temporal concentration course of the/any group taking into account both an actuation point of time, preferably the last actuation point of time, within the locking period and an actuation point of time, preferably the first actuation point of time, after the locking period.

In addition, the control device may be programmed for taking into account the determined temporal individual concentration course, in particular the determined individual PK curve, upon predetermining the amount of one or several of the subsequent application doses.

In several embodiments, the control device of the nasal applicator is further programmed for taking into account the determined target concentration or its minimum value upon predetermining the amount of the next application dose.

Alternatively or additionally, the control device may take into account the determined temporal individual concentration course, in particular the determined individual PK curve.

In some embodiments, the control device is further programmed for limiting the amount of the next application dose and/or for limiting the concentration resulting therefrom.

Alternatively or additionally, the control device is programmed for limiting the cumulative amount of all application doses delivered within a predetermined time period and/or for limiting the concentrations resulting therefrom.

Again alternatively or additionally, the control device is programmed for limiting the cumulative amount of all dispensed application doses and/or for limiting the concentrations resulting therefrom.

For this purpose, the control device optionally accesses data stored in the data storage which data is relating to the substance.

In several embodiments, the nasal applicator according to the present invention is suitable for nasal administration of an application dose of substances which are or comprise, in particular, medical agents.

In other embodiments, the present invention relates to a drug dispensing device which is not a nasal applicator. What is stated herein with respect to the nasal applicator, therefore, also applies without restriction to a drug dispensing device as a nasal applicator, in particular when it is portable and can be operated by the user, e.g. a patient, alone and is usually also operated unaided or without special knowledge.

In particular, a drug dispensing device is suitable for other modes of administration, for example for further transmucosal administration, in particular via the oral mucosa, for example in the cheek pocket (buccal) or under the tongue (sublingual), for intravenous or also for inhalative administration.

In some embodiments, the dispenser is or comprises a particle generator or droplet generator, for example a vortex chamber, a spray head, a nozzle, a cannula, a dropper, a perforated plate, a baffle, a porous membrane, or similar.

In several embodiments, the dispenser comprises an energy application mechanism and/or a conveying mechanism. This may be mechanical and/or electronic, such as a micropump, piston pump, rotary pump, peristaltic pump, centrifugal pump, or similar.

In some embodiments, the dispenser comprises an analog, in particular continuous, and/or a digital (0 or 1) control for dosing. This may be part of the electronic control device of the nasal applicator. In particular, an application dose or dispensed amount of the substance corresponds to an amount in the range between 1 μL to 300 μL per actuation.

In several embodiments, a high degree of reproducibility and accuracy in dispensing the amount may be provided by a user-independent actuation (alternatively: releasing) by always using the same releasing parameters, such as releasing speed, releasing force, acceleration, flow rate, pressure, rotation, stroke, etc.

In some embodiments, the nasal applicator comprises a dose valve.

In certain embodiments, the nasal applicator comprises an elastomer ring, particularly a removable elastomer ring, for sliding it onto a nasal attachment (also known as a nose dip). The elastomer ring serves to provide the most airtight seal possible between the nostril and an exterior.

In some embodiments, the nasal applicator comprises, for example commercially available or conventional, nasal spray cartridges having an integrated conveying device or pumping device, respectively. By this integrated conveying device or pumping device, different dosage sizes may be dispensed after venting (priming), for example by extending or shortening the stroke path of this device.

In some embodiments, a conveying device or pumping device is released mechanically, i.e. manually, by the user/patient.

For this purpose, for example a spring energy may be manually or externally energetically charged, adjusted, and released.

In several embodiments, the dose-detection device of the nasal applicator comprises a sensor configured to determine the dispensed substance or its amount of agent. Said determination of the dose or its volume or amount may be performed, e.g. by direct measurement, e.g. by a flow sensor, etc. and/or by derived measurement. In a derived measurement, the dose is determined or derived by calculation. This may be done, e.g. by measuring revolutions (min−1), angles, stroke path, path, stroke number, time, valve actuations (number and/or time), etc.

Alternatively or additionally, the detection of the dose may be done by a spray sensor, which allows to determine whether droplets of the atomized fluid or aerosol actually form or are formed in the area of the nasal attachment. For example, the spray sensor uses the effect that the atomized fluid or aerosol scatters light and operates as a so-called scattered light sensor.

In some embodiments, the dose-detection device may be integrated in e.g. the control device of the nasal applicator. In other embodiments, it is provided separately.

In several embodiments, the dose detection may be carried out by a flow sensor, a flow-through sensor, or by a droplet distribution measurement and/or particle distribution measurement.

In some embodiments, the droplet distribution and/or particle size distribution may be measured in order to determine the dose.

In several embodiments, the droplet size distribution and/or particle size distribution may be set or adjusted or adapted by the releasing parameters, such as, e.g., releasing speed, releasing force, pressure, acceleration, flow rate etc. This is particularly provided in order to easily adjust the droplet distribution and/or particle size distribution to different agents.

In some embodiments, the data storage of the nasal applicator comprises at least one internal storage component that is non-volatile and can be overwritten or written to multiple times and/or an external storage component that can be replaceable in particular (e.g., SSD memory card, USB memory, HDD, etc.). Alternatively or additionally, a network, a smart device, a charging station (docking station), or the internet may be considered as external storage.

In several embodiments, it may be provided, e.g. as a final step of the procedure released by the control device, to delete the personal data and/or individual control data from the data storage of the nasal applicator and/or to reset the nasal applicator to the factory defaults at the end of the treatment or therapy without, or after, an automatic data backup in or on a (further) internal or external storage device suitable for this purpose. Advantageously, this allows the nasal applicator, or sections thereof, to be delivered or passed on to additional patients after the patient's therapy has ended, without data protection concerns opposing thereto.

In some embodiments, the data storage, in particular the external data storage, may be implemented as a digital pain badge and may be used by the user/patient, for example when traveling or during inspections, to provide proof or evidence to the treating physicians and/or inspectors.

In several embodiments, the user/patient interacting with the nasal applicator may be considered as a pre-programmed, self-regulating system that eliminates the need for programming and eliminates potential sources of error. A similar approach is known from the use of IV-PCA Smartpumps for the administration of opiates. The pre-programmed, self-regulating system encompasses specific dose thresholds (for example dose limits, concentration limits, time limits, limits based on a therapeutic effect, i.e., for example a lower dose for a low pain score). These dose thresholds may be dose limits per time, a specific number of releasings or actuations, limits of a single dose, a daily dose limit, a concentration limit, and/or similar. When the dose limits are reached, two consequences may occur: The dispensing of further application doses may either be paused, or the dose may be reduced so that a therapeutic effect can no longer occur. In particular, however, a placebo effect is still enabled here.

In some embodiments, the user/patient may be considered as a control loop and the releasing behavior of the nasal applicator may be adjusted according to the user/patient response in terms of dose and therapy based on the detected or determined pharmacological data. For this purpose, direct measured values (actual values), e.g. the blood sugar value or the opioid concentration in the blood, may on the one hand be used, or on the other hand, indirect measured values (derived and/or estimated values), e.g. pharmacokinetic (“PK”) curves of a (reference) population may be used. Direct measured values are preferably used to adapt the releasing behavior.

In several embodiments, the electronic control device is further programmed to predetermine a predetermination point of time of an application dose of the substance, taking into account subjective therapeutic observations of a patient-specific therapeutic effect, such as, e.g., pain. These observations may be or encompass the patient's pain level (pain score), pain type, pain duration, pain intensity, and/or pain frequency.

This means that the user/patient determines what is still bearable for them or how much pain they feel. This may be done, for example, by manual input on a one-dimensional rating scale for determining pain intensity. Such scales may be, for example:
a numerical rating scale (NRS, for example from 1 (=pain-free) to 10 (=strongest intensity)),
a verbal rating scale (VRS, with descriptions of pain intensity, for example “none”, “moderate”, “medium”, “severe”, “very severe”, or descriptions of temporal occurrence, for example “never”, “rarely”, “sometimes”, “frequently”, “always”),
visual analog scale (VAS, for example as a line whose endpoints represent extreme states, such as “no pain” and “unbearable pain,” on which the user/patient marks their subjective perception of pain without perceiving an appropriate scale),
functional activity scale (FAS, e.g. with the classifications “no limitation” if the user can perform a certain activity without limitation (e.g. due to pain), “mild limitation”, if the activity can only be performed to a limited extent (e.g. due to pain) and “significant limitation”, if the activity cannot be performed (e.g. due to pain or due to side effects of pain therapy), or
impairment of function such as, e.g., insomnia, impairment of the ability to eat and/or drink, and/or impairment of deep breathing.

In some embodiments, objective and/or subjective observations may be considered. These may encompass, for example:
vital signs (such as blood pressure, heart rate, temperature, etc.),
pupillary light reflex,
delirium
hallucinations
vertigo
tolerance
addiction
increased sensitivity to pain (hyperalgesia)
sedation,
side effects (such as itching, nausea, vomiting, etc.),
physiological observations,
total dose/volume and/or total concentration of substance/agent,
remaining volume in the container,
sleeping pattern or sleep behavior.

In several embodiments, the present invention may be counted among the so-called personalized medicine, in which the user/patient is considered as a pre-programmed, self-regulating system, and in which a therapy as individual as possible is aimed at with or based on dose limits (absolute) and/or dose limits per time, concentration limits and/or quantity limits and/or limitation of the number of releases and/or limitations for a single dose and/or daily dose, etc., as well as alternatives listed and elaborated below. The more input or information on the status of the user/patient is available, the more individualized the therapy can be. In certain embodiments, in particular when concentration limits are considered in this regard, doses of different amounts may result.

In some embodiments, the electronic control device is further programmed for predetermining a predetermination point of time of an application dose of the substance, wherein patient-specific setup data is alternatively or additionally entered and/or considered. These setup data may for example comprise or arise from:
data from the (electronic) patient record,
data from the individual anamnesis,
data from a pre-operative consultation (pre-op),
data collected during surgery (for example during anesthesia),
data collected in a PACU (Post Anaesthesia Care Unit, recovery room, etc.), and/or
individual patient anthropometric data (for example weight, age, height, gender, etc.).

In several embodiments, subjective therapeutic observations, e.g. using a pain score as described herein, may alternatively or additionally be entered and considered.

In some embodiments, the electronic control device is further programmed for predetermining a predetermination point of time of an application dose of the substance by alternatively or additionally considering objective therapeutic observations. These observations may be verified, preferably automatically, by using sensor data for vital signs. The vital signs may encompass for example:
heart rate (HR),
heart rate variability (HRV),
(partial) blood oxygen saturation (SpO2),
blood pressure,
blood pressure trending (BPT),
electrochemical skin reactions,
temperature,
photoplethysmogram (PPG),
electrocardiogram (ECG),
respiratory rate,
blood glucose level,
stress,
carbon monoxide (CO) measurement
electroencephalography (EEG).

In several embodiments, it is possible to personalize, adapt, modify, etc. the method initiated by the control device. This may be done by a user interface, management software and/or via peripheral devices.

In some embodiments, the method initiated by the control device generates value tables for controlling the nasal applicator.

In several embodiments, the method initiated by the control device calculates different releasing parameters for the nasal applicator depending on the position of the device.

In some embodiments, the circadian, ultradian, and/or infradian rhythms of the user/patient may be taken into account.

In several embodiments, the nasal applicator transmits the data from the data storage wired, for example using LAN, Powerline, or PowerLAN, and/or wirelessly using appropriate protocols, for example Wi-Fi, Bluetooth, WLAN, GPS, RFID, NFC, barcode, QR code, ZigBee, Wibree, WiMAX, IrDA, WPAN, infrared, etc., and/or is in signal communication with the data storage.

When a signal communication or communication connection between two components is mentioned herein, this may be understood to mean a connection that exits during use. It may also be understood that a preparation for such a (wired, wireless or otherwise implemented) signal communication exists, for example by coupling both components, for example by pairing, etc.

Pairing is a process to establish an initial link between electronic units for the purpose of communication. The best-known example of this is the establishing of a Bluetooth connection, by which various devices (e.g. smartphone, headphones) are connected to each other. Pairing is sometimes also referred to as bonding.

In some embodiments, the data disclosed herein are transmitted using an interface between the smart device and the computer.

In several embodiments, the data disclosed herein are transmitted in a web-based manner, i.e. no software installation is required.

In some embodiments, the data disclosed herein are transmitted using encrypted data transmission.

In several embodiments, the data disclosed herein are preferably transmitted over an adjustable range.

In some embodiments, low-energy solutions are preferably provided for the nasal applicator and the devices connected thereto in order to consume as little power as possible.

In several embodiments, a fleet management is provided for the nasal applicator and the devices connected thereto, i.e. managing, scheduling, controlling, and monitoring multiple nasal applicators with the connected devices.

In some embodiments, there is a possibility of integrating the nasal applicator including the devices connected thereto into an existing IT infrastructure. Interfaces may be provided for this purpose, e.g., with clinical devices and/or with other medical devices and/or with diagnostic devices and/or with a laboratory, etc.

In several embodiments, interfaces may be provided to electronic patient records and/or to hospital quality reports and/or to national, European and international data collection initiatives or databases. Thus, data from, for example, internal and external research, internal and external survey data (e.g. patient satisfaction, adverse events, etc.) can be accessed. Within the European Union, for example the International Statistical Classification of Diseases and Related Health Problems (ICD-10) is of interest; within Germany, Diagnosis Related Groups (DGRG) and Quality Improvement in Postoperative Pain Therapy (QUIPS) may provide comparative data; and in the United States, Risk Evaluation and Mitigation Strategies (REMS) programs, DEA surveillance programs, and DEA documentation, respectively.

In some embodiments, a modular/expandable dock or docking station is provided for the nasal applicator or the electronic control unit thereof and/or other peripheral devices. The dock or docking station is used to store and/or manage the nasal applicator and/or peripheral devices. The dock or docking station is suitable for storing and/or managing the applicator and/or peripheral devices. For example, the dock may be provided for charging, software updates, pairing, hardware checks and/or releases, etc.

The dock or docking station also counts as a peripheral device in some embodiments. Optionally, however, in several embodiments, the dock or docking station does not serve to transfer data between the dock or docking station on the one hand and a nasal applicator physically in contact with it and/or a server or cloud server on the other hand, while in others it does.

In several embodiments, the dock or docking station is provided as a standalone IT infrastructure, e.g. on the internet or intranet (via hub/router/switch). Alternatively or additionally, the dock is integratable into and/or integrated in an existing IT infrastructure via appropriate interfaces.

In several embodiments of the present invention, the data interfaces are wired, e.g. configured as USB, USB-C, Thunderbolt, LAN, etc.

In some embodiments, it may be provided to gain access to outsourced system intelligence using e.g. IBM Watson, and thus to provide coupling to existing hospital information systems (HIS/PACS) via common interfaces such as HL7 and DICOM. For example, a technician may thus be provided with access to the nasal applicator, to the devices connected to the nasal applicator, to interfaces, and/or to software.

In several embodiments, a central server is provided.

In some embodiments, the nasal applicator, particularly its electronic control device, comprises an energy storage device, particularly a rechargeable battery or accumulator.

In several embodiments, the energy storage device may have an application-related and/or indication-related battery design. In these embodiments, a differently sized energy storage device may be provided depending on the indication, in particular energy storage devices with different service lifetimes or validity periods. For example, a three-day energy supply may be sufficient postoperatively, whereas a longer energy supply (e.g., over two weeks) may be advantageous in the treatment of migraine.

In some embodiments, wireless charging of the energy storage may be provided, for example using pins (e.g. in a charging cradle) or without pins (e.g. Qi, using inductive energy transfer).

In several embodiments, wired charging of the energy storage may be provided, for example using a power supply or using Power over Ethernet (PoE).

In some embodiments, replaceable batteries may be provided as energy storage device, such as disposable batteries (button cells, AA, AAA, AAAA, block batteries, etc.), rechargeable batteries, rechargeable battery packs, etc.

In several embodiments, an external power connection may be provided as energy storage or PoE may be used.

In some embodiments, a solar cell may provide the necessary power. The solar cell may be provided directly on the nasal applicator or, e.g., on a charging dock or charging station.

In several embodiments, the necessary energy may be generated by a winding mechanism, for example as in a clockwork, or by so-called “energy harvesting”, i.e. for example by pressing a button or lever once or several times. The energy generated in this way may be used immediately or later for the application of application doses.

In some embodiments, the substance reservoir of the nasal applicator is provided integrally, in other embodiments it is provided non-integrally, in particular co-packed, i.e. co-packed with the nasal applicator and/or an associated component. In both integral and non-integral embodiments, the substance reservoir is (re)fillable, in particular from the outside.

In several embodiments, the substance reservoir is a pre-filled disposable container and/or an on-site fillable disposable container, e.g. made of glass, plastic, metal, non-ferrous metal, composite materials, etc., or of a combination of these materials.

In some embodiments, the substance reservoir is provided with a position-independent releasing device, i.e. releasing may take place with releasing functionality in all axes and degrees of freedom. This may be implemented, for example by a collapsible and/or an energized receptacle and/or similar.

In several embodiments, the substance reservoir has a constant fill volume, in other embodiments, the substance reservoir has a variable fill volume.

In some embodiments, the substance reservoir may be fillable with and/or suitable for substances free of preservative, and in other embodiments, the substance reservoir may be fillable with and/or suitable for substances containing preservatives.

In several embodiments, the substance reservoir is provided with at least one connection device that serves to connect the substance reservoir to the dispenser, in particular in a releasable manner. This can be done by a Luer connection, a needle, a screw cap, a plug-in connection, a click connection, by pressing or similar. Furthermore, mechanical pins and/or a form-fit connection may also be encompassed by the present invention for connection.

In some embodiments, a device, in particular a barcode/QR code and/or RFID-device, may be provided on the substance reservoir for detecting and/or storing transmittable information for traceability of one or several applications. These may further be or encompass, e.g. a label, a QR code, a chip, an RFID/NFC tag, an image, which may each be scanned, etc., or a combination thereof. Applications include, for example, information about the substance such as name, expiration date, pharmacological data, concentration, etc.

In several embodiments, technical solutions may be employed at the substance reservoir for error prevention in terms of the poka-yoke or lock-and-key principle. For this purpose, for example a label, a QR code, a barcode, a chip, an RFID/NFC tag, an image, which may each be scanned, etc., or a combination thereof may be provided on the substance reservoir. Corresponding control devices and/or computing devices with suitable software may likewise be encompassed by the present invention for this purpose. Thus, among other things, improper handling and/or insertion of an incorrect substance reservoir into the nasal applicator can be advantageously avoided. Mechanical pins and/or a corresponding form fit may likewise serve this purpose and are, therefore, also encompassed by the present invention.

In some embodiments, the substance reservoir is a non-integral, pre-filled, disposable container made of, for example, glass, plastic, metal, non-ferrous metal, composite materials, etc., or any combination thereof, which is stored separately from the nasal applicator, in particular from its dispenser. In particular, this avoids compatibility problems between the material(s) of the substance reservoir of the substance. Separate storage may reduce or even avoid the risk of extractables or leachables migrating into the substance.

In some embodiments, the substance reservoir is a refillable, integrated, reusable container with a filter; in other embodiments, without a filter.

In several embodiments, the substance reservoir is an external reusable container with a filter; in other embodiments, without a filter.

In some embodiments, the nasal applicator comprises an additional substance reservoir, in particular a liquid reservoir, in which a further substance may be received.

Preferably, the further substance is different from the first substance. The further substance may be medical or non-medical, e.g. hyaluronic acid, ectoine, aloe vera, etc.

In some embodiments, the nasal applicator or its substance reservoir comprises multiple fluid chambers, such as the aforementioned second fluid chamber. In such embodiments, the nasal applicator may be suitable to support several therapies, e.g., analgesics or cannabinoids on the one hand, and e.g. insulin on the other hand. In certain embodiments, it may be provided to use a fluid from the second fluid chamber and/or in the substance reservoir to neutralize or destroy the substance in the second fluid chamber and/or in the substance reservoir, or vice versa. This may be particularly advantageous in the event of theft or misuse. For this purpose, e.g. a desiccant may be provided in the second fluid chamber and/or in the substance reservoir, which passes through (similar to the principle of a filter or coffee filter) the substance and thus the agent contained therein or by which the agent or the substance is absorbed (for example, as by a sponge) and the desiccant binds the substance or the agent (e.g., its salt).

The desiccant may be provided e.g. in a filter, in the second fluid chamber, or in a sponge, e.g. as granules.

The desiccant may also be provided elsewhere in the nasal applicator, preferably such that, if desired, the substance containing the agent passes through or is stored in or comes to rest in the desiccant.

Desiccants may be, e.g., sodium sulfate, calcium sulfate or calcium chloride.

The filter may be, e.g., a molecular sieve, a filter fleece or an ion exchanger.

Desiccant, particularly as discussed above, may be additionally or alternatively arranged elsewhere in the housing. Hence, not limited to the second chamber and/or the substance reservoir.

In some embodiments, the nasal applicator comprises a cooling device, suitable for cooling one, several or all of the substance reservoir(s), in particular the contents thereof.

In certain embodiments, the nasal applicator comprises a heating device, suitable for heating one, several or all of the substance reservoir(s), in particular the content or contents thereof, respectively. This is particularly advantageous to prevent freezing of the substance or to prevent degeneration/destruction of the substance due to coldness.

In several embodiments, the control device may be provided as a single-use device, and in other embodiments, as a reusable electronic control device. Hybrid embodiments, in which a section of the control device is reusable and a section is provided as a single-use component, are also encompassed by the present invention.

In some embodiments, the control device collects, stores, monitors, processes, calculates, simulates, regulates, and/or controls one, several, or all functionalities of the nasal applicator.

In some embodiments, the nasal applicator is controlled by the control device through an algorithm (embedded software) loaded on the nasal applicator.

In some embodiments, the dispenser may be controlled separately by the control device or together with the dose-detection device.

In several embodiments, the dose-detection device may be controlled separately by the control device or together with the dispenser.

In some embodiments, the nasal applicator is controlled by the control device through a decentrally stored algorithm, e.g. web-based, via a station (ward) computer or smart devices (non-embedded software).

In several embodiments, the control device is in signal communication with further devices, wirelessly or wired, for example in order to interact with peripheral devices; corresponding transmitting and/or receiving devices may be provided. They may be in signal communication with the control device. Peripheral devices may include, but are not limited to:
Patient-authentication devices,
wristbands,
sensor arrays,
docking stations,
computers (PC),
smart devices,
printers,
other medical devices,
devices in a data center,
hub, router, access point,
server,
(cloud) server.

The system according to the present invention may comprise one or several of the aforementioned and/or further peripheral devices.

In several embodiments, peripheral devices of the nasal applicator function as radio cells for a tracking system and/or for generating a geofence.

A geofence is a virtual fence or a virtual boundary, respectively, that “surrounds” a physical location. Like a real fence, a geofence separates a given location from its surroundings—albeit in a digital manner rather than in the form of a physical barrier. Unlike a real fence, however, a geofence may optionally detect movements within the “fencing or enclosure”.

Such a virtual fence may have any size, shape or coupling to peripheral devices. Even a straight line between two points may be possible and provided.

Geofences are created with the aid of e.g. mapping software and allow the user to draw the virtual fence directly over the desired area and/or to establish a coupling, etc., using GPS, W-LAN, WPAN, RFID, Bluetooth signals and/or using other peripheral devices. The digital boundary consists, e.g., of a number of coordinates for latitude and longitude—and in the case of a circular geofence, it consists only of a point which forms the center and by using W-LAN, WPAN, RFID, Bluetooth signals and/or using other peripherals, establishes a coupling, etc.

Geofences may be used to monitor objects within the virtual fence, e.g., by using W-LAN, WPAN, RFID, Bluetooth signals and/or by using a coupling to other peripheral devices etc., or tracking, etc. and/or are used to define and/or activate certain action for an area. In this way, one can, e.g., immediately recognize when a device such as the drug dispensing device according to the present invention, in particular the nasal applicator, is removed from its place of use. Conversely, virtual fences may also be used to keep objects out of a particular area.

In some embodiments, the electronics of the control device may be designed to be redundant, e.g. dual channel, by a monitoring system/watchdog, etc. In particular, this may advantageously contribute to ensuring safe operation, thereby increasing safety and meeting the various risk classes in medical technology or medical device technology.

In some embodiments, the requesting device of the nasal applicator encompasses a release mechanism, which in turn encompasses at least one of the features described below.

For example, the release mechanism may be purely or substantially mechanical, e.g., similar to an insulin pen or a Respimat® soft mist inhaler.

Alternatively, the release mechanism may be a combination of mechanics (release mechanism) e.g. heart curve, actuator, and/or skip or skip release, and electronics (control).

In several embodiments, the release mechanism may be, or encompass, a unidirectional, bidirectional, multidirectional, rotationally controlled, stepwise controlled, and/or encoder controlled and/or stepless piston and/or an actuator, etc.

Additionally thereto, in some embodiments, a counter-directional dosage adjustment mechanism (cf. insulin pen) may be provided. Alternatively, an electronic dosing (cf. electronic insulin pen) is also encompassed by the present invention.

In some embodiments, the release mechanism comprises a pump, e.g., a micropump, piston pump, rotary pump, peristaltic pump, rotodynamic pump, centrifugal pump, or similar.

In several embodiments, the release mechanism is based on a bidirectional two-stroke principle (suction/pressure), e.g., according to the operating principle of the Respimat®.

In some embodiments, the application device or the release mechanism may cause micro-dosing, e.g., in the range of 1 μL to 300 μL.

In several embodiments, the application device or the release mechanism may be programmed or configured to apply a dosage or dose per time unit, e.g., 100 μL/sec.

In several embodiments, a dispensed amount (application dose) may be easily and/or accurately reproduced because by the present invention the amount or magnitude of the application dose is independent of actuation by the user/patient.

High reproducibility and accuracy of the dispensed amount through user-independent release with always the same releasing parameters, e.g., force, velocity, acceleration, pressure, flow rate, stroke/path, general energy input may advantageously contribute to patient safety.

In some embodiments, the release mechanism may initiate releasing by a mechanical or electronic pulse generator. In these embodiments, no application of force to the system is required.

In several embodiments, the application dose of the substance is adjustable, particularly variable, particularly through embodiments of the release mechanism as described herein.

In several embodiments, the substance is in a pressure cartridge.

In some embodiments, the cloud of droplets is generated using “vibrating mesh”, “piezo”, porous materials, and/or evaporation. The delivery amount may be determined by time or by a pre-measured amount, which is then atomized.

In several embodiments, the release mechanism encompasses a dose valve which e.g. remains open as long as it is held depressed. Also encompassed by the present invention is a dose valve with a dose chamber, a time-controlled valve, or a flow-controlled valve, etc.

In several embodiments, the manually adjustable mechanism for changing the capacity volume of the dose chamber is formed e.g. by a rotary knob, as a ratchet or similar, with, e.g., a numerical scale or indications of the quantity, dose, etc. to be dispensed. Here, the stop of the ratchet may or may not act against an energy storage, e.g., a spring or an elastic element. The release or delivery is released, e.g. manually, analogously in the same way as with a classic nasal spray. After the application is complete, the ratchet slips, for example via a lock nut or similar, into the new initial position of the plug. The application device may or may not be connected to the plug, e.g. by a piston.

In some embodiments, the mechanism is manually adjustable, in others it is automatically adjustable or adjustable in a combination of manually and automatically or semi-automatically. This applies to any embodiment of the mechanism encompassed by the invention.

In several embodiments, the requesting device may be located laterally.

In some embodiments, the “ratchet” may be a conventional ratchet. It may have a counter-rotating thread. Again, this may apply to any mechanism referred to herein.

In several embodiments, the mechanism for varying the capacity volume of the dose chamber may simultaneously load an energy storage, such as in the form of a spring energy, and apply it by the requesting device that releases the energy storage.

In some embodiments, the dose setting or dose application may be a combination of manual and automatic action.

In several embodiments, the substance reservoir also represents the dose chamber. This means that an application is carried out from the substance reservoir directly or indirectly via the nasal attachment, without the application dose to be applied first having been received in a chamber or dose chamber that is fluidically at least temporarily separated from the substance reservoir. An independent loading device is, therefore, not required in these embodiments.

In this way, it may be advantageously ensured that the application device always moves, rotates, conveys in the same direction, which allows to compensate for the inevitable play in a simpler way than if a left-right, up-down movement were performed.

Based on measurement results of an optional flow sensor or an optional device for quantity detection, etc. of any kind, in some embodiments the motor for application may be controlled for example by simply switching off the power supply or giving the controller an appropriate signal, in particular to switch off.

In several embodiments, another design is provided in which, e.g., the application dose or its amount is manually set. For example, an energy storage is manually tensioned and manually released.

In some embodiments, the application device may drive or push on a separate plug; alternatively, the plug may be integrated in the application device.

In several embodiments, in particular for a single-use device, the plug and loading device may be non-releasably connected and/or integrated in each other. In other embodiments, particularly for a multiple-use device, plug and loading device may be two individual parts, or the spindle and plug may be separated or separable from each other.

In some embodiments, the nasal attachment may be integrally formed with the housing, or connected thereto, such as plugged on, screwed on, or similar. A connection site may be provided for this purpose. It may be arranged inside or outside an enclosed lumen of the housing.

In several embodiments, the application device comprises a piston with an end-sided plug that enters the substance reservoir, which in these embodiments may also serve as a dose chamber.

In some embodiments, a servomotor is arranged to shift or adjust a stop, for instance by a spindle or an adjusting screw.

In several embodiments, the stop, which could alternatively be provided to be manually adjusted, limits the travel of a release mechanism. In these embodiments, the mechanism may be understood to be part of the application device.

In some embodiments, it may be provided that the release mechanism is biased by the energy storage of a spring or by another energy storage and released in the biased state, e.g. manually. Alternatively, the release mechanism may be driven and/or released by a motor.

In several embodiments, the stop may be configured by an internally threaded outer ring, which may be slidably provided along an externally threaded cylindrical guide.

In some embodiments, the stop may be provided to be shiftable or adjustable by motor and/or manually.

In several embodiments, the requesting device may be in the form of an end-sided push button which is pressed against a spring.

In some embodiments, a valve may be a check valve or a valve that opens only at a certain pressure, e.g., from 4 bar pressure, and closes again after a pressure drop below 4 bar.

In several embodiments, a filter may be provided at the end of the dose chamber or downstream of the connection site.

In some embodiments, the dose chamber is adjusted by an inclined plane. A suitable mechanism may be provided for this purpose. The release and application may be analogously the same as for a conventional nasal spray. An optional device such as a return spring may return the application device and/or other components to their initial position.

In several embodiments, the setting/adjustment of the mechanism may be carried out manually or automatically by e.g. a motor. In this, the movement may be rotational or linear.

In some embodiments, the substance reservoir may be configured with trailing plug, a pouch bottle (bag-in-bottle), a “bag”, a reservoir with a riser tube, etc.

In several embodiments, the mechanism for changing the capacity volume of the dose chamber may be integrated in the nasal attachment or may be disposed upstream or downstream of the connection site.

In some embodiments, the mechanism for changing the capacity volume is the dose chamber itself.

In some embodiments, the plug comprises, or is the piston, which is driven into the substance reservoir by gas pressurized in a pressure vessel. When the requesting device is actuated or depressed, an application dose as provided or prepared in the dose chamber is dispensed.

In several embodiments, the dose chamber is set by inclined planes or similar, such as stops and similar. When the requesting device is manually released, the gas pushes the piston with the plug forward and into the substance reservoir. In these embodiments, the setting and releasing may also be carried out manually or automatically.

In some embodiments, the inclined planes may be arranged, e.g., linearly or circularly (curvilinear).

In some embodiments, the dose chamber or its volume is set by inclined plane(s) and motor, which could alternatively be done by hand. In these embodiments, the spring energy is manually loaded or preloaded, which could alternatively be done by motor or other energy sources.

In several embodiments, the release is implemented using a spring energy. In these embodiments, the stroke may be set automatically or manually and/or the release may take place, e.g., manually, for example, by rotating the spring energy mechanism manually or automatically.

In some embodiments, this rotation may be implemented using a motor.

In several embodiments, the mechanism for changing the capacity volume of the dose chamber is adjustable for the application dose of the substance.

In several embodiments of the nasal applicator, the latter comprises a pump, in particular a micropump, which is preferably not directly coupled to the vortex chamber, which may be present in the nasal attachment, but fills the dose chamber with the desired delivery amount or application dose from the substance reservoir.

In some embodiments, the pump fills the dose chamber. In some embodiments, the pump may be directly connected to the connection site or to the nasal piece.

In several embodiments, the dose chamber may additionally be adjusted manually, by e.g. a ratchet, or automatically, e.g. by a motor. The pump shuts off or is stopped or turned off, for example, after a certain pressure or time is reached.

In some embodiments, a flow sensor may be used for this purpose, which flow sensor controls the pump.

In several embodiments, the substance introduced into the dose chamber by a pump displaces the piston limiting the dose chamber, thereby increasing the capacity volume of the dose chamber.

In some embodiments, a check valve may limit the dose chamber such that substance may flow out of the substance reservoir into the dose chamber, but cannot flow out again through the check valve.

In several embodiments, the pump, which is or may be part of the loading device, may be operated by motor or alternatively by hand, e.g., by a rotary knob or by a lever. Numerical information, which may be provided on the rotary knob in these embodiments, may indicate the adjusted or adjustable dose amount.

In several embodiments, a spring energy may be loaded or tensioned manually or by the motor using a spring. When said spring mechanism is released, the application amount is forced through the vortex chamber of the nasal attachment.

A release may be provided in some embodiments. It has the function of the requesting device. For example, it may wake up the system from a standby state and reload the next dose (i.e., fill the dose chamber).

In several embodiments, the release may be designed to act mechanically or electrically. For example, the release may be used to release a ratchet or send a signal to the electrical control device, which then releases an actuator.

In some embodiments, a valve, which may or may not be integrated in the check valve, seals the connection site. To fill the dose chamber, this valve is closed and another valve is open. As soon as the application device moves, e.g. towards the connection site, the check valve closes. Now all valves are closed and pressure builds up in the chamber.

In several embodiments, one or several of the existing valves may be either electrically switched, open at a minimum pressure, and/or manually opened via the requesting device.

In certain embodiments, the requesting device may engage different latching or snap-in steps depending on whether, or depending on how far (to what degree or level), the dose chamber is filled.

In some embodiments, a spring may be provided that presses on the plug.

In several embodiments, the housing of the nasal applicator comprises a lid which, when open, exposes the interior of the housing such that, e.g. the substance reservoir, optionally designed as a vial, may be removed from the housing and, for example, replaced by another one.

In some embodiments, for establishing a fluid communication between the dose chamber and the substance reservoir, for example, a needle may be provided which pierces e.g. a septum of a vial.

The pump may again be driven by a motor, or alternatively this may be done manually.

In several embodiments, a motor may be used to drive or operate the pump, the mechanism for changing the capacity volume of the dose chamber, and/or the loading of the spring energy. Thereby, the motor may optionally drive the application mechanism or the application device. All these components may be provided and designed in order to be operated and/or controlled manually or automatically in any combination.

In some embodiments, a check valve is preferably provided at one end area of the piston (application device), which check valve closes upon delivery (piston moves towards cover). At the opposite end area, i.e. near the connection site, a valve is provided which is switchable or closed when the dose chamber is filled by the pump and opens when the piston (application device) moves towards this valve. This may or will be implemented by a switchable valve (electrical or mechanical), which opens while releasing. The same could also be implemented with a check valve which opens only at a certain pressure, e.g. 4 bar. Then, while the dose chamber is filled by the pump, this valve would remain closed until the pressure in the dose chamber has increased above the opening pressure of the valve—similar to a pressure relief valve or pressure sequence valve.

In several embodiments, the pump fills the dose chamber, and, using the resulting pressure increase, the loading device and/or the application device are pushed away from the connection site. In this, e.g., a spring energy storage device is loaded. The energy thus stored is then used to move the application device toward the connection site.

In some embodiments, the motor may drive the pump, the loading device, and/or the application device.

In several embodiments, the orientation of the vial may be rotated about, e.g., 180°.

In some embodiments, the release mechanism further comprises or enables a reversible pumping direction (suction). This may be provided in particular e.g. for discarding and/or neutralization of the substance or agent in the event of misuse and/or theft. Thus, for example an air pump or other conveying mechanism or actuator may be provided which forces the plug of the substance reservoir out of the reservoir, hence, the substance comes into contact with the desiccant, thereby enabling the former to be bound by the latter.

In several embodiments, the housing of the nasal applicator functions as a requesting device for actuating the release mechanism.

In some embodiments, the nasal applicator is suitable and/or adapted for operation by right-handed users, and in others, for operation by left-handed users. In certain embodiments, it is suitable and/or adapted for both left-handed and right-handed operation.

In several embodiments, the release of the releasing mechanism and/or the amount of application dose resulting therefrom are independent of user/patient actuation.

In some embodiments of the nasal applicator, it is provided that the user/patient sets the amount of an application dose to be dispensed themselves. This may be done, for example, based on calculations or instructions from a physician. These can be communicated to the user/patient in various ways, for example via a smartphone or similar. In certain embodiments, software, in particular an application (app), for example for a smartphone, may be encompassed by the present invention for this purpose.

In several embodiments, the requesting device comprises at least one of the following devices for activating the release mechanism, or is designed as such:
an integrated release button, for example designed as soft-touch button, push button, switch, area (button) on a touch screen, sensor or similar;
an integrated input device, for example a keyboard, a touch screen, one rotary knob (or more), an integrated sensor or a camera for capturing biometric data, for example fingerprint, face or voice, code, or similar;
an external input device connected, preferably wirelessly, to the nasal applicator, in particular to its requesting device, for example an application (app), a remote control, input via web or cloud, computer, smart device or similar.

In certain embodiments, the external input device may be designed analogous to the examples of embodiments of the integrated input device mentioned herein.

In some embodiments, a particular behavior of the user/patient may control or influence the requesting device. For example, pressing twice for a release may with the first press cause the nasal applicator to “awaken” possibly associated with a system check, an authorization check, etc., a calculation of the application dose and provision of the same, while the second press may finally cause the application dose to be released.

In several embodiments, the nasal applicator may, particularly through one of its devices, generate an output information, preferably automatically, preferably after a dispensing of a substance dose. This output information may be or encompass a log file of the treatment. In this, each detectable signal or interaction, in particular each actuation, may be logged. Alternatively or additionally, the output information may be or encompass the generation of a standardized and/or personalized report related to the user/patient. The output information may be provided by a digital (online) dashboard, by printing functions (e.g. for the report on controlled substance), by export, etc., to a device suitable for this purpose, respectively, preferably wirelessly; in some embodiments also wired and/or with contact pins.

In several embodiments, the release mechanism of the dispenser may be activated in other ways, for example breath-activated or nose-activated, i.e. the release mechanism is activated, for example automatically by a negative pressure sensor when the nasal applicator is inserted into the nose and the patient/user inhales through the nose. A mechanical implementation of this release mechanism is also encompassed by the present invention.

In some embodiments, optics are integrated into the nasal applicator, for example for capturing biometric data, such as fingerprint or face, capturing a QR code, a barcode, or similar.

In several embodiments, the output information may be or encompass an indication of the amount of time (countdown/timer) until the next release. The indication (or display) or expiration of this time period may for example be visual (e.g. by a colored display, an LED, or push message), acoustic (e.g. by an alarm), tactile (e.g. by vibration), or analog. A colored indication may include for example the nasal applicator or a section thereof changing color to signal its status, such as changing to “green” when a new application may be effected.

In some embodiments, the output information may be or encompass an output of step-by-step instructions (e.g. for a setup or advanced operation or similar) using a visual and/or acoustic medium, for example a smartphone or similar.

In some embodiments, step-by-step instructions are provided on the nasal applicator and/or on corresponding peripheral devices and/or on smart devices.

In several embodiments of the present invention, input options and/or output options may each be provided in a barrier-free manner. This includes e.g. input and/or output in Braille, visual, acoustic or tactile input and output, e.g., vibration, sound, etc. This is also particularly advantageous for use of the present invention in poor visibility conditions and/or at night. In particular, these input options and/or output options may be or encompass contactless input options and/or output options.

In several embodiments, the nasal applicator comprises a QR code reader. This may serve for scanning the UDI (“Unique Device Identification”) of the single-use cartridge, for detecting the single-use housing, the applicator electronics, a single-use wristband and/or wristband electronics thereof, and/or for automated reading in a docking station.

In several embodiments, the present invention also encompasses printer functionality. This may among other things serve to generate a report, to produce stickers (e.g. for the controlled substance and/or patient record), etc.

In some embodiments of the present invention, data access is provided directly by an own input and reading display.

Alternatively or additionally, data access may be provided indirectly by “digital online management software” for example by a smartphone, tablet and/or computer and/or by other interfaces.

In some embodiments, the nasal applicator and/or at least one of its devices comprises touch-sensitive surfaces.

In several embodiments, the present invention may comprise a microphone integrated into the nasal applicator and/or a smartphone microphone. This microphone may be used to record e.g. a pain diary.

In certain embodiments, the present invention may encompass a “shake to wake” function. Devices suitable for this purpose, for example an accelerometer, a gyroscope, an angle sensor, a position sensor and/or similar are also encompassed by the present invention.

In several embodiments, said devices are complementarily provided in order to detect improper handling of the nasal applicator, for example falling, shock, vibration, excessive pressure on the applicator, rough handling, or similar.

In some embodiments, the present invention may comprise a camera integrated into the nasal applicator and/or a smartphone camera. This camera may be used to look, for example, inside the nose, ears, etc., to have a diagnosis made after transmitting (the pictures/images) to a physician, and similar (Digital Health Diagnostics). It may also serve to detect the level of sedation, for example by images of pupillary response of the eye.

Patient authentication means that only the authorized user/patient can operate the device. A user may for example be, among others, a physician, nurse, and/or other authorized persons.

In some embodiments, the nasal applicator comprises an integrated authentication module for this purpose; in other embodiments the authentication module is provided externally; in certain embodiments partially integrated. Here, the authentication mechanism underlying the authentication module is preferably coupled to exactly one single, specific device. Signal transmissions required for this purpose may be provided and performed, e.g., as disclosed herein. Corresponding designs of the signal transmission partners involved may be provided.

In some embodiments, an integrated authentication module, i.e. being provided on or in the nasal applicator, may act optically, e.g., by QR code readers or by devices for recognizing and comparing biometric data; in other embodiments, it may act acoustically, e.g., by voice recognition; in further embodiments, it may act tactilely, e.g., by the input of a secret number or password. In this, the authentication module comprises the sensor arrangement, respectively suitable for authentication, and an evaluation device.

In several embodiments, the authentication module may be provided in order to be compatible with further devices.

In certain embodiments, it may be provided to undergo authentication by vibration (for example a particular vibration pattern) or by speech recognition or voice recognition. In further embodiments, an area in which the device is unlocked may be defined using the GPS location of the nasal applicator and/or using a geofencing function.

In some embodiments, a partially integrated or external authentication module may be provided on the patient, while in other embodiments it is not provided on the patient. In partially integrated or external authentication modules, the authentication mechanism may for example be based on token, wireless authentication. The authentication module may be or encompass a wristwatch, an RFID bracelet, a barcode/QR code, a necklace, a ring, a smartphone, a smartwatch, a tablet, or complementary a software for the aforementioned modules (e.g. an app), etc. In this, the authentication module comprises the sensor arrangement and/or evaluation device being respectively suitable for authentication, analogously as described above.

In several embodiments, the authentication module is compatible with standard hospital patient identification means, for example it may be sticked onto or printed on said means, or it may be integratable therein (e.g. into a patient wristband).

In certain embodiments, the standard hospital patient identification means itself serves as the authentication module, for example the imprinted barcode/QR code.

In some embodiments, it may be necessary to use the authentication module or the authentication of an administrator and/or a user group in order to put the nasal applicator into operation or to cause an application dose to be released or to make adjustments to the nasal applicator.

In some embodiments, the authentication module is provided to additionally authenticate technicians and/or system administrators and to grant them access to the nasal applicator software, particularly its control device.

In several embodiments, it may be necessary to use the authentication module in order to authenticate an administrator and/or a user group in order to remotely operate and/or overwrite data on the control device of the nasal applicator (override function). This may be necessary, for example to initiate extra drug delivery. Authentication may hereby be carried out by any of the devices and/or modes of action mentioned herein.

In some embodiments, the range of the authentication module is variable and/or adjustable, preferably continuously.

In several embodiments, the authentication module may be integrated in so-called smart textiles, for example in eyeglasses, shoes, a patient gown, a pillow and/or pillowcase, and/or a bed cover and/or mattress, a work coat, or similar.

In certain embodiments, the authentication module may also be, or is fastened to the edge of the bed, on a bedside table, in an electrical outlet near the patient.

In some embodiments, the authentication module is a device worn in the ear (in-ear wearable) or similar.

In several embodiments, the authentication module is or comprises a subcutaneous implant for authentication and a sensor arrangement suitable for reading the same.

In some embodiments, the authentication module is or comprises a dental crown or dental splint for authentication and a sensor arrangement suitable for reading the same.

In several embodiments, the authentication module is or comprises a camera integrated in the nasal applicator and/or a smartphone camera for capturing data, for example biometric data, alternatively or additionally a microphone, integrated or external, for the purpose of voice recognition.

In several embodiments, the nasal applicator encompasses a monitoring/surveillance system, wherein monitoring is or comprises the observation of the patient and surveillance additionally encompasses or is the observation of the patient's environment. By using said system, for example the so-called adherence to therapy, i.e. the extent to which the patient's behavior is in accordance with the agreed recommendations/prescriptions of a medical practitioner (compliance/adherence), can be monitored.

In some embodiments, the monitoring/surveillance system encompasses automated drug monitoring in real time. This may e.g. detect a consumed quantity, a remaining quantity, the number of applications remaining, etc., and store them internally or externally via a storage device suitable for this purpose.

In several embodiments, the monitoring/surveillance system encompasses automated therapy monitoring in real time. This may e.g. detect application points of time and application amounts, attempted (unsuccessful) applications during a locking interval, etc., and store them internally or externally via a storage device suitable for this purpose.

In some embodiments, the monitoring/surveillance system comprises automated therapy diary management in real time. This may e.g. patient-specifically detect side effects, mood states, and/or the severity, duration, and/or frequency of (health) complaints, etc., and store them internally or externally via a storage device suitable for this purpose.

In several embodiments, the monitoring/surveillance system may be provided to generate a mobility log.

In several embodiments, the monitoring/surveillance system of the nasal applicator encompasses a combination of the aforementioned real-time monitorings.

Real-time monitoring using the monitoring system and/or surveillance system of the nasal applicator advantageously allows for easy and/or accurate logging of the therapy.

In some embodiments, the control device of the nasal applicator comprises integrated, visual (or optical) and/or acoustic status indicators and/or alarm indicators, for example, LED(s), buzzer(s), beep(s) and/or a display to indicate the status or alarm, in particular by an error code or similar.

In some embodiments, the control device of the nasal applicator comprises external, visual and/or acoustic status indicators and/or alarm indicators, for example, in or on peripheral devices, smart devices and/or station (ward) computers. The external status indications and/or alarm indications may also include LED(s), buzzer(s), beep(s) and/or a display, and may further be provided as a software dashboard (app), push message, or similar.

In some embodiments, the nasal applicator is configured to send automatic messages, in particular a call for help and/or an alarm to third parties, e.g. specific groups of people, physicians, caregivers, rescue coordination center or similar. This may be done particularly in the event of unusual behavior, such as deviations from stored movement profiles or movement patterns, or for example in the event of constant drug requesting, exceeding the maximum permitted amount of drug (dosing limit) or similar, and/or if the vital signs of the user/patient are outside predetermined limits. Alternatively or additionally, such notification may be provided by active actuation of an “emergency button” by the user/patient.

In several embodiments, a “safe” state of the nasal applicator is provided. In particular, this is provided in the “off” state. In the “safe” state, the system does not dispense a dose under any circumstances.

In some embodiments, the nasal applicator comprises patient monitoring sensor technology. The sensor technology is capable of detecting patient-specific data. These data may be or include objective data such as position, movement, and/or vital signs (e.g., HR, HRV, SpO2, blood pressure, electrochemical skin response, temperature, photoplethysmogram (PPG), electrocardiogram (ECG), blood pressure trending (BPT), respiratory rate, stress, etc.) or subjective data detected via patient feedback as described herein.

Such monitoring systems may advantageously increase or optimize therapy adherence and, thus, the therapeutic effect (e.g. by reminders, supporting information, value coupons, etc., status inquiry, suggestions, activity tracker, notification of relatives/acquaintances, etc.).

In several embodiments, it may be provided to supplement the nasal applicator with further functions, such as reminders, further supporting information, gamification (e.g., earning points, earning TV minutes that can be exchanged with hospital administration for access to TV programming, value coupons, etc.), status inquiries, suggestions, activity trackers, notification of relatives/acquaintances, participation in surveys, e.g. online surveys, etc.

In some embodiments, the nasal applicator may be provided to enable so-called social monitoring and communication, i.e. to provide a, preferably bidirectional, means of communication to family, friends, caregivers, etc.

In some embodiments, the nasal applicator may comprise an antidote in addition to the medical substance in a further substance reservoir, suitable for, e.g., reducing or preventing side effects or for avoiding overdosing or similar.

In some embodiments, the nasal applicator is configured to perform a respiratory gas analysis, e.g. capnography, and to, for example, display the result of the analysis, to transmit the result to a peripheral device, and/or to store the result in a storage device suitable for this purpose.

In several embodiments, the nasal applicator may be connected to sensor arrangements and/or evaluation devices by which the position of the user/patient may be detected and stored. The position may detect the patient's positioning, whether they are standing up, have collapsed or fallen, and/or similar.

These sensor arrangements may be provided internally or externally, preferably as set forth herein, for example integrated in smart textiles with integrated sensors. For this purpose, the nasal applicator may comprise further suitable actuators, etc. In this manner, for example a mobility log, etc. may be established.

In some embodiments, the patient monitoring sensor technology or parts thereof may be implanted. In other embodiments, said technology or parts thereof may be integrated in a dental crown or a dental splint or an earring or ear clip.

In several embodiments, the patient monitoring sensor technology, or parts thereof, may detect or make measurable sensitivities or states of the user/patient, such as pain. This may be determined via physical responses, such as skin galvanic, blood pressure change, heart rate change, and/or pupil dilation.

In some embodiments, the nasal applicator comprises a nasal applicator location system. In this, the nasal applicator location system may be configured to locate the nasal applicator, locate the substance, and/or locate the user/patient. This may involve an integrated real-time tracking system, e.g. fleet management or similar, via wireless connectivity (e.g. via Wi-Fi, Bluetooth, WLAN, GPS, GSM, GPRS, Starlink, WPAN, infrared, RFID, NFC, barcode, QR code, ZigBee, Wibree, WiMAX, and/or IrDA, etc.). Advantageously, the nasal applicator tracking system serves for facilitating device management and enhancing patient safety.

In some embodiments, establishing virtual zones or areas can simplify defining or determining the position.

In several embodiments, the nasal applicator comprises an anti-abuse system and/or an anti-theft system.

In some embodiments, the housing of the nasal applicator is designed to be inaccessible, such that access to the, or any, substance is prevented. This may be implemented for example by a single-use housing which would be destroyed if opened.

In several embodiments, the nasal applicator comprises a device for physically securing the nasal applicator in place. This may be designed for example by a Kensington® lock, a security cable or security rope, cable lock, etc.

In some embodiments, common conductors may be provided for data transmission and/or power transmission. Exemplarily mentioned herein is a LAN—cable for data transmission and/or power (POE) with an integrated steel cable.

In some embodiments, the nasal applicator is provided with a predetermined geofence. In these embodiments, the present invention encompasses the possibility to influence the functionality of the nasal applicator outside of a predefined area, for example by preventing a releasing or by neutralizing the agent of the substance. This may be implemented using a software solution and/or wireless connectivity. As discussed herein, peripheral devices may hereby serve as radio cells.

In several embodiments, the nasal applicator may be designed to be childproof. The childproof design may be implemented for example by hardware (e.g. cover, lock and/or safe) and/or by software (e.g. patient authentication etc.).

In some embodiments, peripheral devices of the nasal applicator may be provided with safety closure and/or protection against manipulation, e.g. a wristband with an inserted steel wire, Kevlar, etc.), and in particular, peripheral devices may be continuously adjustable and thus individually adjustable to the user/patient.

In some embodiments, real time location, in particular geofencing, may serve for fleet management and/or locking/unlocking the nasal applicator and/or releasing a neutralization of the agent in the substance.

In some embodiments, the agent in the substance may be neutralized or destroyed, respectively, for example, by a liquid and/or solid and/or gaseous additive, such as a base, an acid, antagonist, gypsum, cement, and/or cold (N₂O≙−89° C. or CO₂≙−78.5° C.), etc.

In several embodiments, a desiccant, for example as disclosed herein, may be provided to be passed through by the substance, wherein the desiccant binds the substance or an ingredient or agent (e.g. a salt) contained herein.

Suitable desiccants may include, for example:
sodium sulfate
calcium sulfate
calcium chloride

In some embodiments, a filter is provided through which the substance may pass, e.g. in the form of a molecular sieve, filter fleece, ion exchanger, or similar.

In several embodiments, the nasal applicator may be configured to be reprocessed, for example between the treatment of two different patients. Reprocessing may be or encompass, e.g. programming or reprogramming, (re)charging or replacing batteries or other energy storage devices as described herein, (re)filling the substance reservoir, and/or inserting a new cartridge. Further, reprocessing may be or encompass a check, for example in the form of a system check, a full or partial disposal of nasal applicator and/or substance, hygiene measures, such as cleaning and/or disinfecting, and/or maintenance, which may additionally include a system update. The control device may optionally be programmed to delete reprocessing steps, e.g. deleting patient data, automatically or upon request, such as after selecting an appropriate menu item in the control menu of the storage device.

In some embodiments, the nasal applicator is a completely single-use product that must be completely disposed of according to applicable regulations after initial use on the patient. In this, the nasal applicator, the substance(s), and/or any possible peripheral device may need to be disposed of individually.

In several embodiments, the nasal applicator is partially a single-use product and comprises, for example reusable electronics, single-use components that have come into contact with the substance and/or the user/patient (e.g. a single-use housing for the electronics, a single-use nasal attachment, a single-use pump, or similar.

In some embodiments, the nasal applicator may be configured to encompass automated and/or logged disposal of the residual amount of the substance. This may be done for example by pumping into hazardous waste or into a special container or separate receptacle for drug disposal, suction and neutralization, etc. In certain embodiments, this may serve to document the drug disposal.

In several embodiments, the nasal applicator comprises a modular, preferably expandable, docking station for the electronic control device and/or other peripheral devices. For example, the docking station may be configured to charge the energy storage device, transfer software updates to the nasal applicator and/or the peripheral devices, pair any two devices encompassed by the nasal applicator, perform hardware checks, implement releases to the nasal applicator/peripheral device, etc.

In some embodiments, the nasal applicator is configured to automatically (re)order the substance(s).

In several embodiments, the protective cap of the nasal applicator includes antibacterial agents in order to keep the nasal attachment clean.

In some embodiments, the nasal applicator includes a rinsing device for the nasal attachment, as otherwise the volume of substance in the nasal attachment would “stagnate” until the next release, which could promote colonization with microorganisms. This may preferably be provided for sensitive agents in the substance.

In several embodiments, a priming process may be provided during which, e.g., the substance is returned into the substance reservoir and automatically collected. In some embodiments, it may be provided that the nasal applicator blocks releasing if the priming process is not completed.

In some embodiments, an acoustic signal may be emitted during the priming process. This signal may be a warning tone that sounds for example while the priming process is not completed. Likewise, only with or after the end of the priming process an acoustic signal, e.g., as set forth herein, may be emitted.

If reference is made herein, and in particular above, to an acoustic signal, this is not intended to be misleading about the fact that a visual (or optical) signal may also be provided, in particular in accordance with the explanations for the acoustic signal. Thus, a visual signal may indicate that the priming process is not yet completed. Alternatively or additionally, a visual signal may indicate that the priming process is now, or only now, considered completed.

In several embodiments, data evaluation via software (e.g., data export, dashboard, reports, e-mails, notifications, screen, app, etc.) may be provided within and/or outside the nasal applicator.

In some embodiments, data evaluation may provide for applying data encryption. For example, this may be, or encompass, encryption key, blockchain, password, passphrase, token, biometrics, and/or dongle, etc.

In several embodiments of the nasal applicator, further functionalities are connectable, for example a dashboard (control station) from which the devices of the nasal applicator can be centrally controlled, monitored, etc., for therapy and/or further devices and or the application of further substances and/or functions for patient management and/or similar.

In some embodiments, the nasal applicator may be provided or programmed to conduct internal and/or external surveys, e.g., with regard to patient satisfaction, side effects, or the like. These surveys may be standardized, for example according to the International Statistical Classification of Diseases and Related Health Problems (ICD-10, EU) or according to Diagnosis Related Groups (DGRG) and/or according to the Quality Improvement in Postoperative Pain Therapy (QUIPS) (Germany) and or according to the Risk Evaluation and Mitigation Strategies (REMS) programs, DEA surveillance programs, or DEA documentation (USA).

In several embodiments, the nasal applicator encompasses a QIUPS interface (questionnaire, informational material, etc.) to determine the acute and chronic pain experience of the user/patient.

In some embodiments, the nasal applicator encompasses patient information (online and/or offline) for training and/or aftercare of the user/patient, or a possibility to do same. This may be provided, for example, by an online video consultation, by a virtual assistant, by an instruction manual, a patient information leaflet or package insert, information on side effects, FAQs, operation and/or product information, forms, surveys, therapy diary, electronic patient record, etc.

In some embodiments, the nasal applicator encompasses a training function (online and/or offline) for physicians and/or caregivers, which is fulfilled, e.g. by reminders, new trainings, studies, device updates, new functionalities, substances, etc.

In several embodiments, customized reports may be created using the present invention in order to meet the individual requirements of hospitals or other facilities and/or regulations. These may include, for example, Food and Drug Administration (FDA) regulations in the U.S., Federal Institute for Drugs and Medical Devices (BfArM) regulations in Germany, or European Medicines Agency (EMA) regulations in Europe, etc.

In some embodiments, the nasal applicator is provided to support IoT (Internet of Things), for example, by healthcare tracking, identification/authentication, data collection/data mining, and/or sensing. Using communication devices suitable for this purpose, in particular without human intervention, data may then be transmitted via the network. For this purpose, it may optionally be provided to supply one or more of the individual devices and/or peripheral devices of the nasal applicator with one-to-one identifiers so that the individual devices and peripheral devices can recognize each other in a network.

In several of the embodiments, one of the peripheral devices is programmed to receive first data from a first of the nasal applicators, process the first data, and based on the result of the processing, create second data and send the latter to a second of the nasal applicators and/or to the first nasal applicator. In this, second data may be, e.g., an algorithm, or results which were obtained when running the algorithm.

In particular, experience gained during the use of the specific, first nasal applicator on a first user, and which may be reflected in the first data, may be used advantageously in this way to improve the later use of this first nasal applicator. It is encompassed by the present invention that for this purpose, based on the data received from the first nasal applicator second data may be generated by one of the peripheral devices after analysis, editing, processing etc. of the first data and may be delivered, e.g., from said peripheral device to the first nasal applicator. Second data may be associations of the specific first user to a group of users, i.e., for example, encompassing data regarding their classification or regarding the classification of their clinical picture or their user behavior. They may be provided and transmitted as formulas, algorithms, or in other forms. They may be different from a pure update, which is done without considering the specific user, their behavior and/or their characterization as a patient, but, e.g., as a medically indistinguishable consumer.

In particular, experience being gained during the use of the specific, first nasal applicator on a first user and which may be reflected in the first data, may advantageously be used in this way to improve the later use of a second nasal applicator. It is encompassed by the present invention that for this purpose, based on the data received from the first nasal applicator second data may be generated by one of the peripheral devices after analysis, editing, processing etc. of the first data and may be delivered, e.g., from said peripheral device to the first nasal applicator. Second data may be realized as set forth above. It may serve the user of the second nasal applicator to advantageously use experience gained, e.g. or inter alia, by the use of the first nasal applicator by the first user, for their future use of their second nasal applicator.

In some embodiments, it is provided that the use of a nasal applicator will initially begin with a “base algorithm”.

The initial algorithm, also referred to herein as the “base algorithm”, optionally begins with a preset model and a set of parameters for the corresponding indication, which may be initially adjusted based on e.g. age, co-medication, significant pre-existing conditions, etc. After the begin of the treatment or use, the “base algorithm” (model and/or parameters) is trained on the basis of the applied doses and/or the resulting concentrations and preferably with the aid of the comparison between theoretically calculated next application points of time and the actual next application points of time, the actuation behavior of the user and/or the input of vital signs in order to characterize the patient. Based thereon, the use by this patient or treatment of this patient may thus be optimized for further use of their nasal applicator.

For this purpose, procedures, methods and technologies of artificial intelligence and/or machine learning (or the special case of machine learning, the deep learning) may be applied in order to train the “base algorithm” (model and/or parameters), which may take place, for example, on the peripheral device such as by recourse to data that may have been obtained on a plurality of uses of different nasal applicators.

The treatment data thus generated (applied doses, application points of time, vital signs, etc.) or the “training evolution” of the “base algorithm” (model and/or parameters) of each individual user may be collected in a structured manner and homogenized at the end of the therapy or in real time in corresponding databases. This database may then be searched with the help of “Big Data” analysis methods (e.g. cluster analysis, data mining, curve analysis, pattern analysis, etc.) and/or procedures, methods and technologies of artificial intelligence and/or machine learning (or the special case of machine learning, deep learning) for similarities, patterns, abnormalities, etc. with the aim of deriving or evaluating meaningful and implementable improvements/insights for the “base algorithm” and implementing them by transmitting second data.

In an optional step, the “own” database may be networked with other databases, such as e.g. long-term patient data, e.g. EHR (electronic health record), EMR (electronic medical record) and/or HIS (healthcare information system) and/or gene databases and/or the combination of genotype with phenotypes and/or clinical studies and/or long-term studies, e.g. QUIPS (quality improvement in postoperative pain therapy), Pain-Out, etc. and/or “wearables & sensors” data and/or further computer models such as in-silico medicine computer simulations and/or other sources. The goal of this networking is to increase or to expand, respectively, the data situation/base (amount) for the “Big Data” analysis methods and/or the AI methods and the resulting results with which the “base algorithm” is then “trained”. The goal is to then use the “trained new base algorithm” for future treatments. It can be used as second data and be transmitted accordingly. Alternatively or additionally, second data based on such a “trained new base algorithm” may be generated on the peripheral device and delivered to a nasal applicator. This process may be customized for any indication.

In several embodiments, the nasal attachment of the nasal applicator may be configured to measure temperature, humidity, airflow, negative pressure, etc.

In some embodiments of the nasal applicator, said nasal applicator may be configured to perform recordings of sleep patterns, and based on this recording, adjust the continuing doses (dosing schedule) before/during/after sleep, etc.

In some embodiments of the nasal applicator, its housing surface may have a beading effect (lotus effect), i.e., it may be provided to be self-cleaning, antibacterial, etc.

In several embodiments of the nasal applicator, the housing thereof may be coolable, e.g., for insulin. A cooling device may be provided, as well as heat insulating materials or provisions.

In certain embodiments of the nasal applicator, the housing thereof may be heatable to prevent freezing, flocculation, or degeneration of the agent. A heating device may be provided, as well as heat insulating materials or provisions.

In some embodiments of the nasal applicator, the housing of the nasal applicator may comprise insulation against cold, heat, moisture, and/or liquid.

In several embodiments of the nasal applicator, additional modules may be clickable thereto. Corresponding holders, devices, etc. may be provided on the nasal applicator.

In some embodiments of the nasal applicator, it may be connectable to a smartphone, particularly the user/patient's smartphone, via the AUX socket or Lightning socket. In these embodiments, the smartphone represents the electronics and the corresponding app represents the required software of the nasal applicator.

Herein applies that in addition to a wire-based signal transmission, as e.g. “wired”, a “cordless” variant, i.e., a wireless variant, is also encompassed by the present invention whenever a signal transmission is disclosed herein.

In certain embodiments, the nasal applicator and/or one of the peripheral devices are programmed to be able to provide a positioning signal, getting onto one's feet-signal, falling signal, etc. Accessories such as smartwatches, stowable and/or bondable sensors, and textiles or smart textiles with integrated sensors, actuators, haptics, etc., mobility protocol, etc., may be provided for this purpose.

The nasal applicator and/or one of the peripheral devices may be provided and configured to perform these functionalities. They may be used to provide a signal to a third party (e.g. to a control center, to the ward room, etc., for instance after a fall of the user), but they may also be used to predetermine the amount of one or several of the subsequent application doses. For example, it may be provided to make the dispensing of an application dose and/or the amount thereof dependent on the user having gotten up in the meantime that is, e.g., having left the bed after all for at least a few steps or some physical activity.

In several embodiments, the nasal applicator is configured to self-mix, particularly automatically, a concentration of the substance and/or to fill the internal substance reservoir and/or to dilute with a substance from a second substance reservoir, e.g., dilute 100 μg/100 μL to 50 μg/100 μL.

In some embodiments, the nasal applicator is suitable for energy harvesting, e.g., by one or several rotation(s) at the bottom of the housing. Thus, a hybrid system between mechanical and electronic systems may be generated wherein the required energy is generated as needed, respectively.

In several embodiments, the control device of the nasal applicator is further programmed to take into account the predetermined target concentration C_{ED} or its minimum value and/or the determined temporal individual concentration course, in particular the determined individual PK curve PK_{Ind}, upon predetermining the amount of the next application dose Dₙ.

In some embodiments, the control device is further programmed to take into account the determined target concentration C_{ED} or its minimum value and/or the determined temporal individual concentration course, in particular the determined individual PK curve PK_{Ind}, upon predetermining the amount of the next application dose Dₙ.

In several embodiments, the control device is further programmed to limit the amount of the next application dose Dₙ and/or the concentration resulting therefrom; to limit the cumulative amount of all application doses D₀, D₁, D₂, . . . , Dₙ₋₁, Dₙ, Dₙ₊₁, . . . , dispensed within a predetermined time period and/or the concentrations resulting therefrom; and/or to limit the cumulative amount of all dispensed application doses D₀, D₁, D₂, . . . , Dₙ₋₁, Dₙ, Dₙ₊₁, . . . and/or the concentration resulting therefrom, wherein the control device for this purpose optionally makes use of data relating to the substance which data is stored in the data storage.

In several embodiments, one of the peripheral devices is programmed in order to receive first data from a first of the nasal applicators, process the first data, and based on the result of the processing, send second data to a second of the nasal applicators.

In several embodiments, one of the peripheral devices is programmed in order to receive first data from a first of the nasal applicators, process the first data, and send second data to the first nasal applicator based on the result of the processing.

One or several of the advantages mentioned herein may be achievable by some embodiments of the present invention, which advantages include the following:

Drug therapy is complicated by the fact that patients respond very differently to the same dose of the same substance. Differences are found in efficacy, as well as in the spectrum and severity of adverse reactions and side effects.

A significant part of the interindividual variability (i.e., differences between different patients) of individual drugs may be attributed to the genetic disposition (pharmacogenetics) of the patient. Identical dosing for different patients leads to very different concentration-time curves (pharmacokinetics) and thus to very different effects (pharmacodynamics), and thus dose individualization is advantageous. In addition to interindividual variability, intraindividual variability, i.e. differences in an individual patient, should be taken into account in a drug therapy. The present invention may contribute to this.

Particularly in the case of multiple administration of substances with narrow therapeutic range, long elimination phase and side effects, the accumulation effect (accumulation or enrichment of the substance) over the administration of several, successive application doses must be taken into account. The present invention may allow a safe application of single or multiple such application doses by the user themselves.

The possibility for the user to carry out the application themselves applies in particular for elderly people, for whom a correct or individually adapted dose plays a decisive role. The present invention may make a contribution to this.

The careful “titration” (which takes place in a determination phase or finding phase) to a substance concentration and the adjustment of the different application doses or the therapeutic effect while avoiding “overshooting”, and the associated avoidance of undesirable side effects can represent a further advantage.

For this purpose, regulating to the target concentration (C_{ED}) may be advantageous. In the case of pain therapy, reaching the target concentration represents a state in which the patient is satisfied with their pain therapy. A concentration exceeding the target concentration no longer contributes to the therapeutic effect, but only increases the side effects to be avoided.

The present invention may allow to solve application-related problems of nasal applicators, such as the different nasal anatomy, the nasal condition, the application angle, how deep the individual user usually inserts the nasal attachment and the like: according to the present invention, a preset amount of the application dose, which effect in the body depends on the aforementioned and other factors, is not necessary. The present invention is suitable for addressing such, above all interindividual, peculiarities in the predetermination of the amount of application doses. Thus, the desired therapeutic effect be realized with certainty.

Disadvantages of other administration forms can be avoided if the substance is applied nasally (i.e. non-invasively), as also encompassed by the present invention. In the case of oral administration, for example, the substance must first be metabolized via the liver (first-pass effect) before it can exert its effect. Other disadvantages are the relatively slow onset of action and the difficulty of administering individual doses. Symptoms such as dysphagia and dry mouth complicate oral administration. For sublingual administration, for example the relatively small absorption surface and accidental swallowing are problematic. The effect is also affected by drinking, eating, or talking too soon after the sublingual application.

Intranasal administration using the nasal applicator according to the present invention is painless, easily self-administered, and has a rapid effect.

The substance-related blocking of further applications during a locking period, which is moreover variable and, according to the present invention, adjustable in begin and duration by the locking device, may allow the target concentration to be found in good time and may advantageously prevent excessive administration of the substance. The locking device ensures that the applied dose can establish its full effect and, thereby, the effect may be evaluated by the user.

According to the present invention, a self-regulating system, amongst others, is thus advantageously proposed which determines or dispenses, after a “titration phase” (which is a determination phase or finding phase) based on application doses that have already been dispensed, the user's actuation behavior and the past time intervals, individualized or personalized application doses, in the case of multiple dosing, which correspond to the needs of the user, or brings about the desired therapeutic effect, respectively.

According to the present invention, the user's body is used as a control loop or controlled system, since only the user's body reflects what the substance does to the body (pharmacodynamics) and the respective application dose is determined and may be applied individually for each user upon request/need.

According to the present invention, it is further possible to categorize the user using the properties of pharmacokinetics and to track the temporal course of the concentration in the body. After an initial “titration phase”, it is possible to assign for the corresponding user whether they tend to the mean value or to “−SD” or to “+SD” within, for example, the group of pharmacokinetics and/or to define their individual concentration-time curve. Since the target concentration, at which the desired therapeutic effect occurs, may also be determined according to the present invention, the present invention advantageously contributes to the further development of personalized medicine.

Interindividual variability (e.g. age, pre-existing conditions, drug consumption) and intraindividual variability (e.g. manipulation, mobilization, circadian progression, improvement in disease state or recovery) may be advantageously taken into account by the control device.

An action by the physician or other authorized personnel is hereby advantageously not required, hence, saving time and resources.

Finally, the present invention may contribute to the fact that advantageously a smaller amount of substance is required to achieve the same effect.

“Interception” of the residual medication, e.g. from the operating room, which may have been delivered as the initial or beginning dose, is also advantageously possible according to the present invention.

In the following, the invention is described based on exemplary embodiments thereof with reference to the accompanying drawing. In the figures, the following applies:

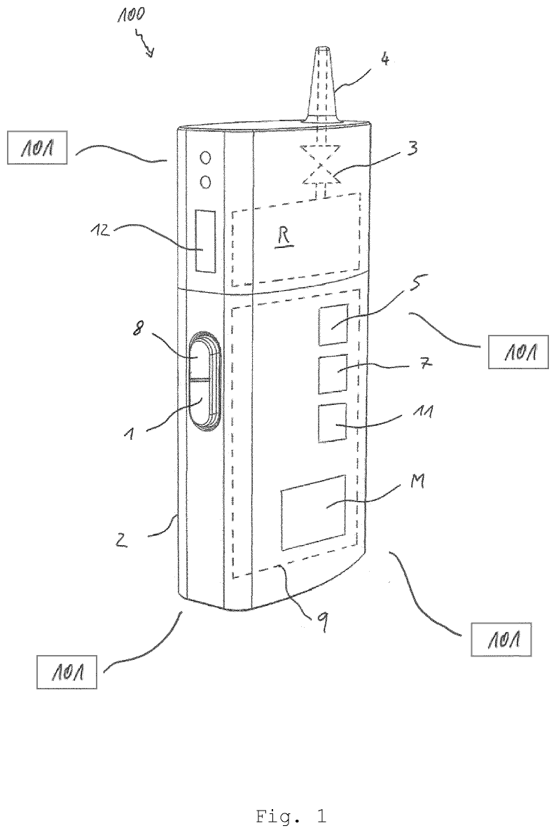

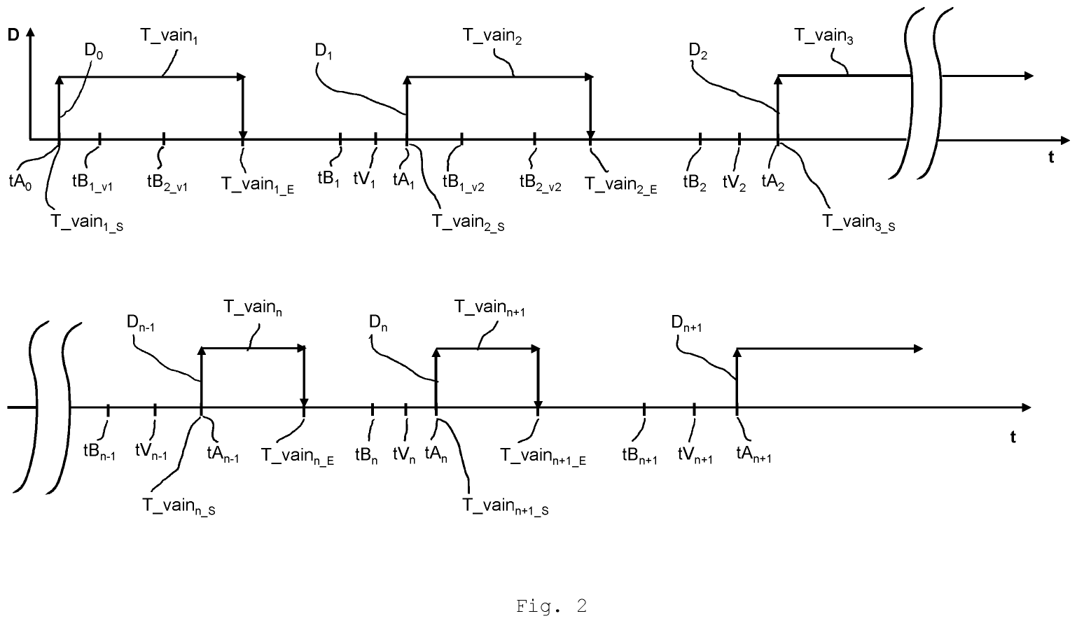

FIG. 1 shows a nasal applicator as an example of a drug dispensing device of a first exemplary embodiment, as well as a system according to the present invention;

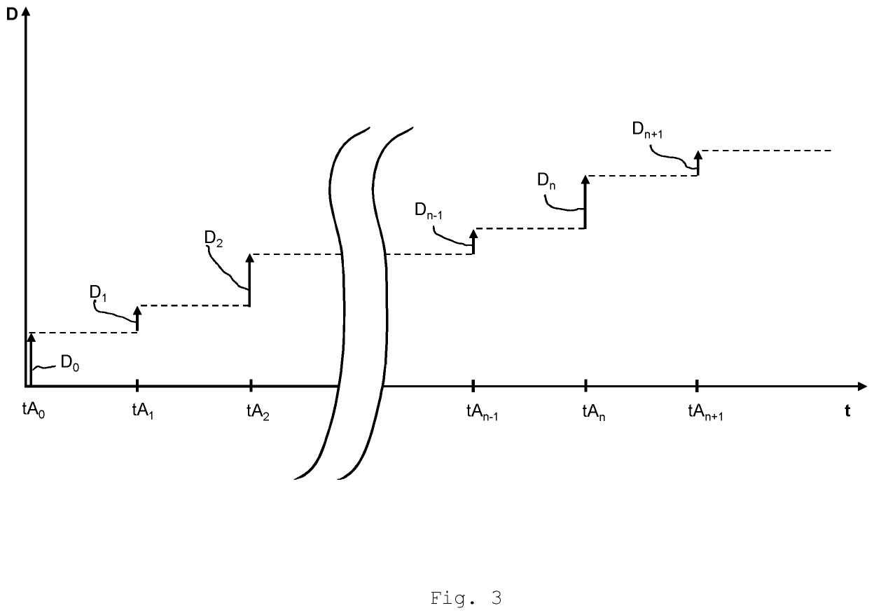

FIG. 2 shows schematically and exemplarily a possible temporal course of a use of the nasal applicator according to the present invention as an example of a drug dispensing device;

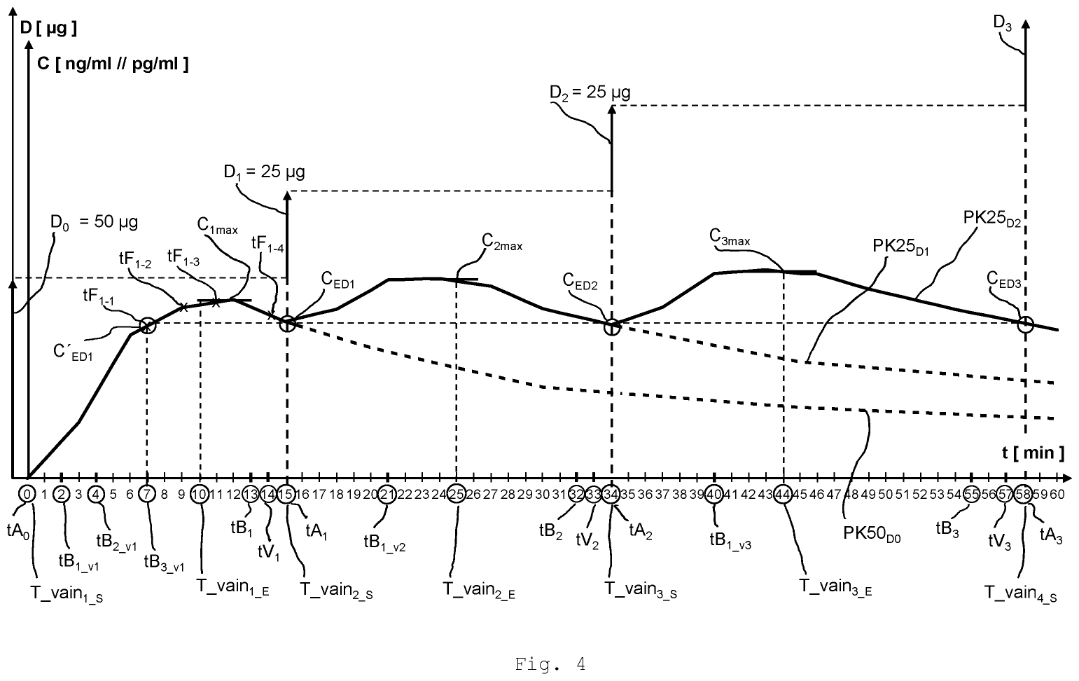

FIG. 3 shows schematically and exemplarily the amount of the cumulative doses of different applications of a substance to the user;

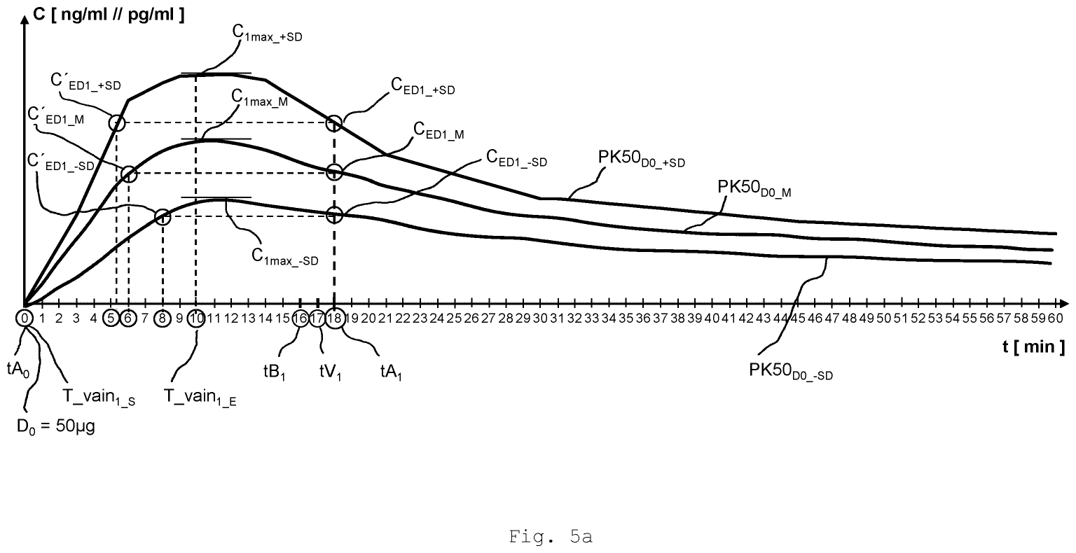

FIG. 4 shows schematically and exemplarily the result of a predetermination of the amount of the different application doses;

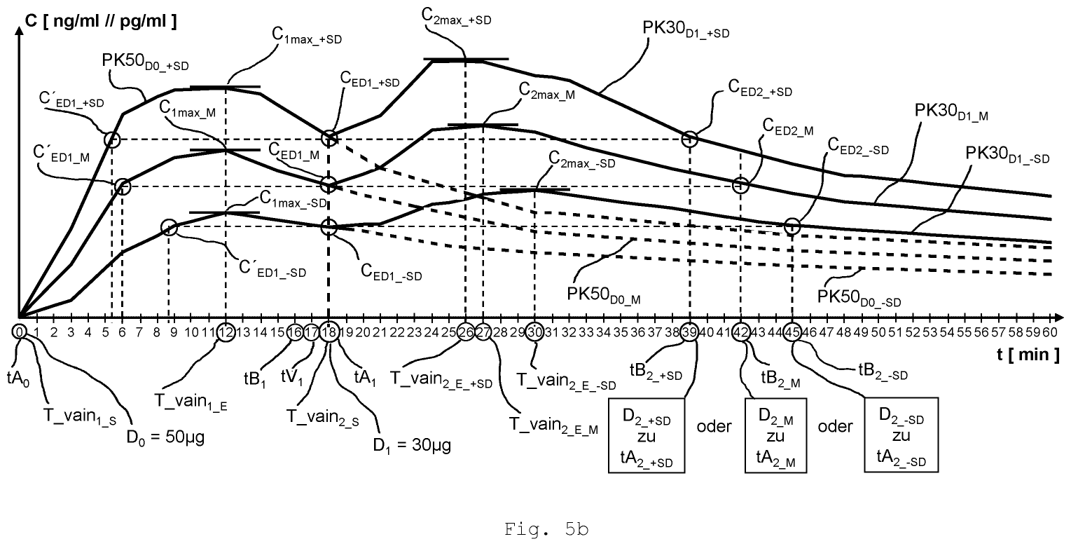

FIG. 5a shows schematically and based on three exemplarily selected temporal concentration courses, the determining of an individual concentration course for a considered user of the nasal applicator according to the present invention

FIG. 5b shows a continuation of the idea of FIG. 5a underlying in some embodiments the present invention; and

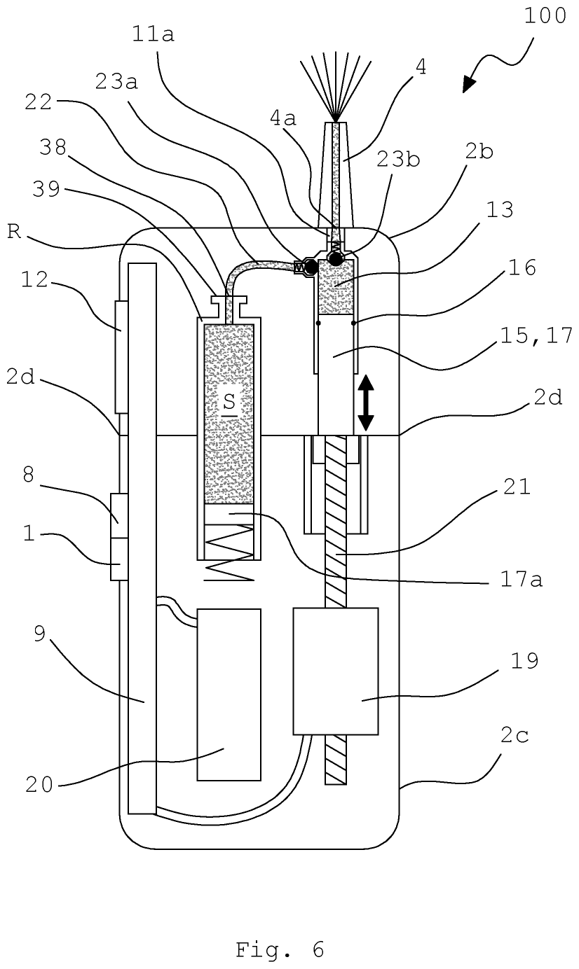

FIG. 6 shows a nasal applicator of a further exemplary embodiment.

FIG. 1 shows a nasal applicator 100 of a first exemplary embodiment in a schematically simplified representation.

As shown in FIG. 1, the nasal applicator 100 comprises a housing 2 in or on which optionally one, all or several of the devices or components mentioned below may be independently arranged. In other embodiments, one or several of these components may in any combination be present externally, i.e., not within the housing 2.

The nasal applicator 100 comprises a requesting device 1, which the user has to actuate in order to be able to apply to themselves a dose D (see FIG. 2 or 3) of a substance S from a substance reservoir R. The substance S may be a medical or non-medical agent, in particular an analgesic. The requesting device 1 may at the same time be, or comprise, an optionally provided feedback device 8. The feedback device 8, if provided, may alternatively be a stand-alone device.

If the user has actuated the requesting device 1, which may be a switch, a button, an insert or similar, and thus triggered an actuation signal, a dispenser 3 may release a quantity of substance S which has been dosed by the dispenser, i.e. determined in terms of quantity or volume. It may flow, be injected, or similar, out of the substance reservoir R via the lumen of a nasal attachment 4 which is to be inserted into a nostril into the user's nose. It may alternatively be applied out of the substance reservoir R via a droplet generator which may be part of the nasal attachment 4 or another or different device of the nasal applicator 100 into the nose.

A display 12, e.g. display, LED, etc., in particular optical or haptic, can be provided.

In addition or supplementary to an optical display, an acoustic indicator, such as a loudspeaker, buzzer, beeper, a haptic indicator, e.g. by vibration, or similar may be provided.

For this purpose, the substance reservoir R may optionally be acted upon by an energy loading mechanism or by another application device, see e.g. FIG. 6. This may work with pressure, force, speed, oscillation, vibration, rotation, electrical/magnetic forces, etc. The latter forces or physical events may, for example, influence the release speed, the force, the pressure, the acceleration, the flow rate, and thus the parameters of the application device, e.g., a droplet generator, i.e., influence the result of the droplet generator. The droplet generator, which may for example be part of the dispenser 3 or the nasal attachment 4, may thus for example be a pressurization mechanism acting on the substance S; for example, a piston pushes into the substance reservoir R, or a pump draws from the substance reservoir R and loads the substance S with pressure, force, speed, etc. and pushes it through the droplet generator.

The optional droplet generator may work mechanically and/or electrically. It may be designed e.g. as a nozzle, vaporizer, piezo element, etc., or comprise similar. It may preferably generate a spray or a cloud of droplets or a cloud of particles.

If such a release by the dispenser 3 is not possible at any points in time and not due to each actuation of the requesting device 1 and/or at any points in time thereof, a locking device 5 may optionally be provided. It is preferably configured to prevent, not allow, etc., preferably time-controlled, a release or a discharge or a delivery by the dispenser 3 during a defined locking period (see, for example, T_vainₙ in FIG. 2) determined according to predetermined criteria with respect to its duration and/or the point of time of its begin and/or end, even if the requesting device 1 should be actuated during the locking period. During the locking period, which in several embodiments is of fixed or invariable duration, while in others it is variable or changeable, an actuation of the requesting device 1 generally or in principle does not lead to an application, whereas this need not to, or does not, apply to an actuation after the locking period has expired.

The amount or volume of that part of the substance S which is delivered and/or has been delivered in individual initial doses or application doses (D₀, D₁, D₂, . . . , Dₙ, . . . in FIG. 2 or in FIG. 3) as explained below at different dispensing points of time tA₀, tA₁, tA₂, . . . , tAₙ, . . . may be detected by an optional dose-detection device 7.

The dose-detection device 7 may be configured to measure, determine, store, sum up, etc., the individual initial doses or application doses D₀, D₁, D₂, . . . , Dₙ, . . . , which have been delivered by the nasal applicator 100 and which are variable, i.e. not fixed dose amounts.

The dose-detection device 7 may, for example, measure a quantity, a volume or another parameter which allows a direct statement about the respective application dose. The dose-detection device 7 may additionally or alternatively measure or determine a parameter which allows an indirect statement about the respective application dose, such as a flow per time, a number of strokes of a pump, a number of revolutions of a pump, or similar.

The dose-detection device 7 or another device, such as the electronic control device 9, may be configured to detect for example the release parameters e.g. the speed, acceleration, force, displacement, etc. Such detected or determined values are optionally stored.

Dose limits and/or concentration limits, which restrict a total dose and/or total concentration limited or predetermined e.g. in relation to a unit of time, can optionally be provided.

The dose-detection device 7 may, if present, additionally or alternatively be programmed to record a point in time at which one or more specific application doses were or are administered by the nasal applicator 100. The dose-detection device 7 may further be additionally or alternatively programmed to detect a time period, for example between two or more dispensing points of time tA₀, tA₁, tA₂, . . . , tAₙ, . . . (see FIG. 2 or FIG. 3) of administered initial doses or application doses D₀, D₁, D₂, . . . , Dₙ, . . . . Further periods of time between any points of time mentioned herein may also be detected (e.g., tA₁−/“minus” T_vain_{1_E}, tA₁− (in particular last) tB_{_v}, tB_{1_v1}−tA₀, tB_{_v} (in particular the last one, i.e. tB_{_v-1})−tA₀, T_vain_{1_E}−last tB_{_v}, T_vain_{1_E}−tB_{1_v1}, whose parameters are explained below).

An electronic control device 9 can optionally be provided in order to control, regulate and/or monitor the function of the nasal applicator 100 or several, any or all of the devices or components mentioned with reference to FIG. 1. It may be provided within the housing 2 or externally, as set forth above. Purely exemplarily, it may be the recipient of information about initial doses or application doses D₀, D₁, D₂, . . . , Dₙ, . . . , which have been predetermined externally. Corresponding wired or wireless signal communications may be provided, uni-directional or bi- or multi-directional, between the control device 9 and the component or components of the nasal applicator 100

An optional detection device 11 may be provided in order to detect whether, that and/or when the user, who may be herein a user, operator or patient, attempted or initiated to apply themselves a further application dose by actuating the requesting device 1.

In each embodiment, the substance reservoir R may optionally be interchangeable, for example, as a single-shot reservoir, a substance reservoir having sufficient substance S for a plurality of application procedures, etc.

The substance reservoir R may be for example pre-filled, self-fillable, integrated, replaceable, designed for single application, and/or designed for multiple application.

The substance reservoir R may be arranged inside the housing 2 or externally thereto.

The substance S may have any state of aggregation (liquid, gaseous, solid, etc.).

An optional data storage M for storing data, or with data stored herein, may be arranged inside the housing 2 or externally thereto.

These data may relate to the previous use of the nasal applicator 100 by the user, as well as time data for the points of time or time periods (synonymously: time intervals) mentioned herein, etc.; they may be the result of an evaluation by the control device 9, etc., or optionally be data for pharmacological, pharmacodynamic, pharmacometric and/or pharmacokinetic data concerning the substance S and/or underlying medical circumstances such as the basic disease, etc. They may be substance-related and/or defined by the physician or other authorized persons, e.g., a tₘₐₓ value of substance S, a physician-corrected, so-called “overruled” tₘₐₓ value, e.g., the point of time or the end of the time interval after application at which or in which the maximum concentration or blood concentration occurs, the half-life, the terminal half-life, a value adjusted by the physician or by other authorized persons, etc.

The data may be objective and/or subjective.

The data may be objective values (sensor values), e.g. blood glucose level, blood pressure, heart rate, respiratory rate, oxygen saturation, etc.

The data may be subjective values (“effect values”), which cannot be measured directly. They are derived based on collected values such as pain score, sedation score, side effects, etc.) and/or measured values (e.g., SpO₂, HR, HRV, etc.).

Data may be, or encompass, recommended concentration, contraindications, side effects, expiration date, and/or package insert, professional or expert information, patient information, Summary of Product Characteristics (SmPC), assembly instructions, disassembly instructions, operating instructions/manuals, etc.

The data may optionally include, in addition to pharmacological and/or pharmacokinetic data, user-related data such as weight, age, gender, BMI, pre-existing conditions, comedications, comorbidities, allergies, etc. Generally, the user history and/or treatment history are encompassed (e.g., data on prior interventions, procedures, diagnoses, medical histories, tests, medications received/prescribed, anesthetics received, muscle relaxants, antiemetics, etc.).

Such and other data may help the optional control device 9 to calculate more accurate dosages, or to increase the safety of the therapy.

Such and other data may relate to release parameters, e.g. those required to obtain a defined droplet size distribution.

Trigger parameters may vary depending on the predetermined dose. It may be set for example by the releasing speed of the application device.

The optional data storage M may be a hardware memory device, a flash memory or an external data storage, for example arranged in a cloud, in a mobile device or the like and/or be in signal communication with further components of the nasal applicator 100 via Wi-Fi/Bluetooth, etc.

The locking device 5 and/or the detection device 11 are preferably components realized or implemented electronically, for example by the control device 9.

A timer or chronometer, such as a clock, may be provided. The individual components, as mentioned above, may make use of it.

By using the chronometer, for example an absolute time measurement (i.e., based on the correct/real/current time) may be optionally carried out or a relative time measurement, in which, preferably when the nasal applicator is initialized or switched on for the first time, a current time stamp is detected, by which the time may be determined back.

It may be provided in some embodiments to inform the user about the expiration of the current locking period, for example visually or acoustically or haptically, such as by vibration.

Since a number of peripheral devices 101 are indicated in FIG. 1, this figure also shows a system according to the present invention.

The peripheral devices 101 may have the same design or configuration, or, at least several of them, may differ from each other.

For example, some of the peripheral devices 101 may be nasal applicators optionally configured as the nasal applicator 100, and/or configured as a docking station, a server, a smart device, etc., preferably as described herein.

Optionally, some or all of the peripheral devices 101 may or may not communicate with or among each other in any combination.

In FIG. 1, it is indicated that they may all also communicate with the nasal applicator 100, but this is also optional. In some embodiments, only some of the peripheral devices 101 can do this.

Further, it may optionally be provided that the nasal applicator 100 and/or the peripheral devices 101 may communicate externally via the nasal applicator 100 and/or the docking station and/or another peripheral device 101.

The substance reservoir R may be connected by, e.g., Luer lock, pin, needle, spike with or without aeration, etc.

A filter (e.g., a 22 μm particle filter), e.g., made of or containing activated carbon, plastic fibers, or similar, or a porous membrane (e.g., a Porex® filter) may be provided. This filter may be provided at the outlet of the dose chamber. More than one filter may be provided.

The substance reservoir R may comprise a plug that can move up. This enables a position-independent release by the nasal applicator 100.

All materials may be inert or partially inert, e.g. made of or with silicone, glass, cyclo olefin polymer (COP), cycloolefin copolymers (COC), etc.

The substance reservoir R may have a pierceable membrane, a screw connection, a push-fit/in connection, a quick connection, etc.

The substance reservoir may comprise a riser tube.

The substance reservoir R may be e.g. a pouch bottle (bag-in-bottle), a “bag” (as in infusions) or another container which may collapse and does not require air to flow into the container. With such solutions, one may release not depending on the position, i.e. in any spatial orientation of the nasal applicator 100.

FIG. 2 shows schematically and exemplarily a possible temporal course of a use of the nasal applicator 100 according to the present invention.

If the user actuates the requesting device 1 in order to apply to themselves, for example, an application dose Dₙ referred to herein as the “next” application dose, then it is determined by the electronic control device 9 or by its detection device 11 whether or not this point of time at which the user wishes to apply to themselves this next application dose Dₙ possibly falls within a locking period T_vainₙ which is still ongoing.

If this time, at which the requesting device 1 is actuated, falls within the locking period T_vainₙ, then no application dose Dₙ is applied to the user at said time in any case. Such points of time are shown in FIG. 2 with reference to an earlier locking period T_vain₁ exemplarily as points of time tB_{1_v1} and tB_{2_v1} with reference to further locking periods, e.g. as tB_{1_v2}, and are optionally included in the predetermination.

If, on the other hand, the point of time of actuation of the requesting device 1 does not fall within the locking period T_vainₙ, but is after the expiration or end T_vain_{n_E} of the locking period T_vainₙ, as shown in the example of FIG. 2 on the basis of the actuation point of time tBₙ, then the next application dose Dₙ is applied to the patient at a point of time designated herein as the next dispensing point of time tAₙ.

Prior to the application of the next application dose Dₙ, its amount is defined or predetermined by the electronic control device 9 at a, herein designated as next, predetermination point of time tVₙ; corresponding calculations may have been performed by the control device 9 itself or it may have accepted results of such calculations.

The predetermination of the amount of the next application dose Dₙ is determined by the nasal applicator 100 or its components mentioned with regard to FIG. 1, such as the control device 9, regardless of whether they are included in the housing 2 of the nasal applicator 100 or are external thereto.

The first predetermination point of time tVₙ, like the actuation point of time tBₙ, may lie at or before the first dispensing time tAₙ; it may therefore coincide with the first dispensing point of time tAₙ, at least within the scope of what is technically possible, so that there is sufficient time remaining between the first predetermination point of time tVₙ and the next dispensing point of time tAₙ for actually predetermining or calculating the amount of the next application dose Dₙ and time for addressing the devices or components, to which the predetermined amount before application must be communicated by signal or in some other way.

Upon predetermining the amount of the next application dose Dₙ, which is an example of the variable dose D, i.e. it is just not or not exclusively preset for the nasal applicator 100 in an unchangeable manner by physician or other authorized persons, manufacturer or similar, then data readable from the data storage M and/or data determined by the components of the nasal applicator 100 mentioned in FIG. 1 may be taken into account.

This data preferably already includes an initial point of time tA₀ prior to the successful actuation point of time tBₙ, at which point of time an initial dose D₀ may possibly have been administered to the user even before delivery of the first application dose D₁, whether by the nasal applicator 100 according to the present invention, by medical personnel, e.g. while still in a post-operative recovery room, etc. Further, this data may include the amount of this optional initial dose D₀.

In the present case, it is assumed that an initial dose D₀ was delivered to the user at an initial point of time tA₀.

This data preferably includes any initial time tA₀, tA₁, tA₂, tAₙ₋₁, prior to the successful actuation point of time tBₙ, at which an initial dose or application dose D₀, D₁, D₂, Dₙ₋₁ had already been administered to the user or at which they had administered a dose to themselves. Alternatively or additionally, the time elapsed since then and preferably also the respective amount of the above-mentioned doses are also included.

When considering the planned delivery of the next application dose Dₙ, these data include, for example, at least the amount of the previous application dose Dₙ₋₁ and, optionally, its dispensing point of time tAₙ₋₁ or the time elapsed since then.

The predetermination, which takes place at the first predetermination point of time tVₙ, thus results in the amount of the next application dose Dₙ. There is determined the amount at the next predetermination point of time tVₙ, which is after the next actuation point of time tBₙ and is not preset by a physician or other authorized persons, manufacturer or similar, which may also apply to all or some of the further application doses mentioned herein. The predetermination at the next predetermination point of time tVₙ is performed taking into account data read from the data storage M.

After the next application dose Dₙ has been applied, a locking period T_vainₙ is set and/or begun again, preferably at the dispensing point of time tAₙ of the next application Dₙ. It may have the same length as previous or subsequent locking periods, or deviate from them. The length of the respective locking period may be predetermined and/or set, for example, by the physician or other authorized persons. When setting the next locking period, a logic or an algorithm may be followed for which it may be calculated at each predetermination point of time tVₙ.

As can be seen from FIG. 2, the procedure described above for this figure may be repeated.

Upon predetermining the amount of an application dose Dₙ₊₁ after the next one, in addition to the amounts and/or the dispensing points of time of the aforementioned application doses and possibly also of the initial dose D₀, the amount and/or the application time t_{An} of the application dose Dₙ designated herein as the next application dose, but then already applied, may now however optionally be taken into account for the first time, wherein the amount and/or the application time t_{An} of the application dose Dₙ may be detected in the meantime as data and likewise kept in the data storage M.

The predetermination of the amount of the application dose Dₙ₊₁ after the next one, which takes place at the predetermination point of time tVₙ₊₁, may therefore now also take into account the length of the time span that lies between the dispensing point of time tAₙ₊₁ after the next one and the next dispensing point of time tAₙ as well as the lengths of the time spans that lie between the dispensing point of time tAₙ₊₁ after the next one and the initial time tA₀ or earlier dispensing points of time tA₁, tA₂.

FIG. 3 shows schematically and exemplarily the applied individual doses D₀, D₁, D₂, . . . , Dₙ₋₁, Dₙ, Dₙ₊₁, . . . and the doses of substance S accumulated over the course of time t, whose amounts and/or dispensing points of time tA₀, tA₁, tA₂, . . . , tAₙ₋₁, tAₙ, tAₙ₊₁, . . . etc. may be included in the predetermination of respective later application doses.

The time axis t as well as its scaling correspond to the one used in FIG. 2 or may be assumed to be identical. The point of time tA₁ and the point of time tA₂ thus correspond to the absolute points of time tA₁ and tA₂ shown in FIG. 2.

FIG. 4 describes schematically and exemplarily the phase of supplying the substance S to the user, also referred to herein as the “titration phase” (which is a finding phase or a determination phase), in which the target concentration prompting the therapeutic effect being desired by the user is determined. The amount of the following application doses may then be determined thereupon and/or an individual, dose- or concentration-based treatment plan may be set up in a time-controlled manner.

Curves such as those shown in FIG. 4 or elsewhere herein may be generated based on, for example, the discussion by Dubois, A., Bertrand, J., & Mentré, F. (2011) in “Mathematical expressions of the pharmacokinetic and pharmacodynamic models implemented in the PFIM software.” UMR738, INSERM, Paris Diderot University. The control device may be programmed to generate such curves and/or to perform the calculations required therefor. The contents of that publication are hereby made by reference the subject-matter of the present disclosure as well.

FIG. 4 shows schematically and by exemplarily the result of a predetermination of the amount of the various application doses D₁, D₂ and further, each of which may be an example of the respective next application dose Dₙ as used herein.

In order to predetermine the respective application dose D₁, D₂, and others, in the embodiment discussed herein, data from the data storage M is used, which comprises pharmacological, pharmacodynamic, pharmacometric and/or pharmacokinetic data. The data may be models, in particular PK models, as well as PK curves. They are preferably patient-specific.

These pharmacological, pharmacodynamic, pharmacometric and/or pharmacokinetic data or models include, in the example of FIG. 4, temporal courses of concentrations of the substance S in the body of the user after the respective applied doses, referred to herein as PK curves. The curves shown reflect the temporal course of the concentration of a specific substance S resulting from the applied dose and the amount of the applied doses. The curves also reflect the processes of absorption, distribution, metabolization and elimination in the body. Such pharmacokinetics models follow statistical distributions, e.g., taking into account those with mean and standard deviation, and are mostly patient-specific, i.e., they may run differently from patient A to patient B, which is why they may also be stored in the data storage M for the different user or user collectives according to different statistical distributions. Since the course of such PK curves depends on the way of administration and the dose, the two PK curves marked with reference numerals in FIG. 4 are designated as PK50 for the intranasal administration of 50 μg fentanyl on the one hand and as PK25 for the intranasal administration of 25 μg fentanyl on the other.

In the example of FIG. 4, pharmacological, pharmacodynamic, pharmacometric and/or pharmacokinetic data, as they may be stored in the data storage M, optionally also include information on the substance S such as tₘₐₓ, which may indicate the point in time at which the highest concentration C(t) is present in the body, for example in the blood, after application of a dose D. The duration of tₘₐₓ may be used as a locking period, at least initially or subsequent to a first dose, in this case the initial dose D₀. Likewise, the locking period may be based on tₘₐₓ in that the locking period is e.g. only 90% of tₘₐₓ, or is determined to be shorter or longer than tₘₐₓ by a number of minutes, etc. Likewise, values above 100% of tₘₐₓ are possible, e.g., 110% of tₘₐₓ, which is after tₘₐₓ, if one wishes to figure in a delay in which the administered substance passes from the blood to the site of action, or if tₘₐₓ is 13 minutes, but the locking period is to be set at e.g. 10 minutes for practical reasons.

The electronic control device 9 may, therefore, be programmed to read out also such data stored in the data storage M relating to the substance S and to take this into account upon predetermining the next application dose Dₙ of substance S.

As shown schematically and exemplarily in FIG. 4, there was initially administered to the user an initial dose D₀ of 50 μg of intranasal fentanyl at the initial point of time tA₀. The concentration floods up to a first maximum C₁ₘₐₓ, which is why the locking period end T_vain_{1_E} of the first locking period T_vain₁ is also defined for this point of time, whose locking period begin T_vain₁_s is at tA₀.

If only the initial dose D₀ were applied to the user, the concentration C(t) would decrease over time and, assuming the PK curve PK50 shown in dashed lines in FIG. 4, would be halved after approximately 30 minutes.

However, since such a halving obviously does not meet the needs of the user who has actuated the requesting device 1 at the actuation point of time tB₁, a first application dose D1 in the amount of, for example, 25 μg fentanyl is administered to them at the first dispensing point of time tA₁. The predetermination of this amount carried out at the predetermination point of time tV₁ has shown that 25 μg fentanyl is sufficient, given the course of the PK50 curve, to achieve a concentration in the interplay between decreasing effect or concentration of the initial dose D₀ and simultaneously increasing effect or concentration of the first application dose D₁, which, on the one hand, does not exceed the initial dose D₀ of 50 μg fentanyl, which was initially considered as sufficient, and, on the other hand, does not fall below a desired therapeutic effect-producing concentration C_{ED} of about 45 μg fentanyl.

The concentration C_{ED} of about 45 μg fentanyl prompting the therapeutic effect desired by the user is recognized by the control device 9 in that it recognizes that the user has obviously missed the desired therapeutic effect from the actuation point of time tB₁, which is only shortly before the first dispensing point of time tA₁, which has caused him to actuate the requesting device 1.

As FIG. 4 shows, the user attempts to request two further applications during the locking period T_vain₁ (at the points of time tB_{1_v1} and tB_{2_v1}), since the desired therapeutic effect has apparently not occurred or has not yet occurred. These applications are refused because the concentration of the initial dose D₀ has not yet reached its maximum, but in any event the locking period T_vain₁ has not yet ended. It will reach its maximum only towards the end of the locking period T_vain_{1_E}. Since it can now be seen that the actuation point of time tB₁ does not occur until some time after the end of the locking period T_vain_{1_E}, the system may use the PK curve to derive a concentration at which the desired therapeutic effect occurs. In this case, an initial dose D₀ of 50 μg fentanyl results in a therapeutic time window of 8 minutes (from minute 7 to minute 15).

It can also be seen in FIG. 4 that the concentration for the desired therapeutic effect is already reached at minute 7. This can be seen from the fact that from this point on, no further futile actuation points of time tBₘᵥn occur during the locking period T_vain₁. As can be seen, however, the maximum concentration of the initial dose D₀ has not yet reached its maximum. One can use the concentration prevailing at the last futile actuation point of time tB_{3_v1} for verification by matching this prevailing concentration with the expected actuation point of time after T_vain_{1_E}.

Knowing the concentration C_{ED} prompting the desired therapeutic effect from the first actuation point of time tB₁, the control device 9 may now independently predetermine the amount of the first application dose D₁. It may predetermine an amount of 25 μg fentanyl, knowing that the decay of the concentration C(t) due to the initial dose D₀ and the simultaneous increase of the concentration C(t) due to the first application dose D₁ will lead to a second maximum C₂ₘₐₓ, which will not be above a therapeutically acceptable maximum.

Using the pharmacokinetic models (PK curves), the control device 9 may optionally calculate a time when the concentration of the dose D₁ will fall below the desired therapeutic effect. In FIG. 4, this takes place after another 19 minutes or 34 minutes since tA₀.

If the concentration C_{ED} prompting the desired therapeutic effect and/or the respective next maximum Cₙₘₐₓ is known from the above considerations, the respective next locking period may be calculated from the knowledge of the point of time at which the concentration C_{ED} or the respective next maximum Cₙₘₐₓ will occur or will be reached. For example, the locking period T_vain₁ may be taken from the data storage M in connection with the amount of the initial dose D₀. The end T_vain₂_E of the following, second locking period T_vain₂, may however be obtained from the knowledge of the time of C₂ₘₐₓ, both may be identical to each other. Alternatively, optionally at the time, tA₁, at which the concentration C_{ED} is achieved, the point in time at which the concentration C(t) will have dropped again to the concentration C_{ED} may already be calculated. Half of the difference between these two points of time, as well as another part of it (e.g., 40%, 35%, etc.), may be set as the duration of the respective next locking period.

Alternative approaches are also included. In general, the calculated time for the next fall below C_{ED} could be selected as the locking period, or shortly before. This would be the case in FIG. 4 at 58 minutes from tA₀. So, the control device 9 could set the locking period to the next target concentration C_{ED} (at minute 58 in FIG. 4) at tA₂, here i.e. tV₂.

The amounts of the further application doses, e.g. D₂ (in this exemplary case 25 μg fentanyl each) would be predetermined following similar considerations as for D₁: they were determined taking into account the amount of previously administered application doses as well as the time elapsed since their application; for the second application dose D₂, a residual concentration attributable to the initial dose D₀ and the first application dose D₁ was taken as a basis; for the third application dose, not shown here, the residual concentration attributable to the initial dose D₀, the first application dose D₁ and the second application dose D₂ would be or have been taken as a basis. A residual dose is thereby co-determined by the amount of the respective previous application as well as the time elapsed since its application.

The amount of the next application dose Dₙ may be predetermined such that the concentration in the body, in particular in the blood, of the user will only drop again to the target concentration C_{ED} after a predetermined time period has elapsed, which may be adjusted e.g. by the physician or other authorized persons, beginning from the dispensing point of time tAₙ of the next application dose Dₙ.

As FIG. 4 shows, the user actuates the requesting device 1 at actuation points of time tB_{1_v1}, tB_{2_v1} and tB_{3_v1}, i.e., during the locking period T_vain₁. However, since these three actuations lie within the locking period T_vain₁, the user cannot obtain an application dose D therewith. Nevertheless, the control device 9 records the three unsuccessful actuation attempts and their actuation points of time. In the example of FIG. 4, they are at minute 2 (tB_{1_v1}) and at minute 4 (tB_{2_v1}) and at minute 7 (tB_{3_v1}), respectively, and may optionally be stored in the data storage M. If the unsuccessful actuation attempts are analyzed for this, such as the time interval between them, when they stop, etc., then, to a first but sufficient approximation, the concentration C_{ED} which is desired by the user and which produces the intended therapeutic effect may be detected or defined.

In FIG. 4, feedback points of time tF_{1_1}, tF_{1_2}, tF_{1_3} and tF_{1_4} are indicated. They stand for moments in which the user actuates a feedback device 8 to indicate using feedback that they are satisfied or not satisfied with the felt effect of the respective concentration present at the time of feedback. Said user may thus signal to the nasal applicator, for example, that they are currently free of pain, that they would not prefer a higher concentration in the body, etc.

In FIG. 4, one can see in the PK50_{D0} curve that there is a “straight line” from about minute 45, i.e. the slope remains constant (pseudo-equilibrium “Lambda”).

The elimination half-time is now the time in which the prevailing concentration at minute 45 is halved.

According to the literature, the elimination half-time of fentanyl ranges from 3 to 12 hours, depending on habituation.

FIG. 5a shows exemplarily and schematically using three exemplary PK-curves PK50_{D0_+SD}, PK50_{D0_M} and PK50_{D0_-SD} the statistical distribution (mean and standard deviation) of the possible concentration courses in the user's body after administration of 50 μg intranasal fentanyl, which were obtained from a sufficiently large population. There may be more than just three curves lying between the thresholds shown, i.e. the corresponding study of the collective/population may have resulted in more than just three PK curves distinguishable from each other. All of them, or the models on which they are based, may be stored in the data storage M and/or taken into account by the control device 9. For the sake of simplicity only three PK curves are shown and discussed here.

In general, equal doses of the substance may lead to different concentrations of C(t) in the body depending on the individual patient. This is influenced by various factors, such as pharmacogenetics, intraindividuality, age, weight, gender, previous medication, previous diseases, etc. Furthermore, different nasal anatomies, the nasal condition, the angle of application, how deep the nasal attachment is inserted and how the manually triggered nasal spray is operated may play a role in the course of the respective PK curve, which influences the concentration C(t) in the body over time.

By using population pharmacokinetic models such as the PK curves shown in FIG. 5a, PK50_{D0_+SD} (mean plus one standard deviation), PK50_{D0_M} (mean), and PK50_{D0_-SD} (mean minus one standard deviation), such differences may be mapped.

As can be seen, the same dose of 50 μg intranasal fentanyl leads to three different concentration maxima C_{1max_-SD}, C_{1max_M} and C_{1max_+SD} for these three exemplarily selected curves. A plurality of other curves could also be displayed. They would in turn have different concentration maxima. The time until the maximum concentration does not differ in the three curves shown and also not in those not shown from the plurality of curves, or if it does, only marginally. The end T_vain₁_E of the locking period T_vain₁, which is oriented to the time at which the concentration maximum C_{1max_-SD}, C_{1max_M} and C_{1max_+SD} is reached, therefore, ends at the same time for each of the three curves of FIG. 5a.

If one assumes that the initial dose D₀(D₀=50 μg) is applied at the point of time tA₀, then the control device 9 may calculate the maximum concentrations C_{1max_-SD}, C_{1max_M} and C_{1max_+SD} (and many more) using the population pharmacokinetic models. However, since the time to reach these maximum concentrations is the same for all variants, the control device 9 sets the first locking period (T_vain₁, see FIG. 5a) for all three PK curves equal to the time until the maximum concentration is reached, which in this case is set to 10 minutes as an example. Reaching the maximum concentration may take place in a range. In the present case of intranasal fentanyl, it is between 10 and 12.8 minutes. Since the increase in concentration in the range of 10 to 12.8 minutes is very small, one rounds the time to a practical measure such as, e.g., 10 minutes in this case. Of course, one could also choose 11, 12 or 13 minutes. Thus, if at tA₀ the initial dose D₀ (D₀=50 g) is applied, the respectively resulting maximum concentration is calculated, the flood time until reaching the maximum concentration C_{1max_-SD}, C_{1max_M} or C_{1max_+SD} is calculated, the length of the locking period T_vain₁ (here 10 min) is calculated and T_vain_{1_S} and T_vain_{1_E} are set.

According to the different courses of the three PK curves PK50_{D0_+SD}, PK50_{D0_M} and PK50_{D0_-SD}, one would, at the first actuation point of time tB₁, at which the concentration falls below the concentration prompting the therapeutic effect desired for the user, at which the patient would receive the first application dose D₁ upon request (points of time concerning actuation by the patient, predetermination etc. are not shown here for reasons of simplification or fall together with the first dispensing point of time, which may actually be the case in practice), calculate (or read off with a view to the PK curves plotted as in FIG. 5a) a different concentration C_{ED1_+SD}, C_{ED1_M}, C_{ED1_-SD} respectively, which respectively prompts the desired therapeutic effect. This concentration is also referred to herein as the target concentration or C_{ED}. Its value is based, among other things, on pharmacodynamics.

It should be noted that the actuation point of time tBₙ may correspond to the predetermination point of time tVₙ and/or the dispensing point of time tA₁. Thus, for example, the following may apply: tB₁=tV₁=tA₁.

FIG. 5a shows that the concentration C_{ED1_+SD}, C_{ED1_M}, C_{ED1_-SD} had already been present before the dispensing point of time t_{A1} as C′_{ED1_+SD}, C′_{ED1_M}, C′_{ED1_-SD} at 5, 6 and 8 min, respectively. This may also be derived by the omitted tB_{_v}'s.

The foregoing is the basis for an embodiment discussed with reference to FIG. 5b, in which the nasal applicator 100 considers, in a specific manner, the user's actuation behavior.

If one draws a line (straight line through two points) in FIG. 5b beginning from tA₀ to the corresponding concentrations C_{1max_-SD}, C_{1max_M} und C_{1max_+SD}, one obtains three straight lines, each with a different slope. If one now looks at the increase in concentration on the basis of the time intervals, one could already make an assessment of which curve the user might be assigned to, because the PK50_{D0_+SD} curve has the greatest increase in concentration in the same time interval and the PK50_{D0_-SD} curve the smallest. Since the desired therapeutic effect depends on the concentration, one can make an initial assessment.

FIG. 5b schematically shows the exemplary procedure for determining, selecting and/or defining an individual PK curve PK_{Ind}, which represents the agent concentration course of 50 μg intranasal fentanyl of a concrete, individual user. It should be noted at this point that the target in determining an individual temporal concentration course, as used herein, need not always be an individual PK curve of the user. According to the present invention, it may also be sufficient if, for example, the statistical mean value curve PK50_{D0_M} is used as an orientation, or if threshold curves such as the “mean plus standard deviation” curve or the “mean minus standard deviation” curve or others are approached and the range in which the user moves is known.

FIG. 5b encompasses the illustration of FIG. 5a, but supplements it from the first dispensing point of time tA₁ with the administration of a first application dose D₁ in the amount of, for example, 30 μg fentanyl.

From the first dispensing point of time tA₁, the PK curves PK30_{D1_+SD}, PK30_{D1_M} and PK30_{D1_-SD} assumed before this time are applied respectively. Based on the target concentrations C_{ED1_+SD}, C_{ED1_M}, C_{ED1_-SD} determined in FIG. 5b, the control device 9 or the algorithm determines a plurality of possible doses and concentrations C(t) derived therefrom, which allow an assessment/categorization, i.e. when the expected points of time of the next actuation for requesting a further application dose would have to occur. In this case, the system has defined 30 μg, and the system cumulates the previous curves and, based on the determined target concentrations C_{ED1_+SD}, C_{ED1_M}, C_{ED1_-SD}, projects the respective curve courses into the future. It is determined at which point of time the target concentrations C_{ED2_+SD}, C_{ED2_M}, C_{ED2_-SD} should be mathematically undercut fall below. In the example of FIG. 5b, they are marked tB_{2_+SD}, tB_{2_M} and tB_{2_-SD}. Based on this check, the control device 9 may determine in which range or on which curve the user is present. The user may be better classified via these and other iterations.

Three possible cases are considered below:

Case 1: If the population pharmacokinetics curve PK30_{D1_M} (mean value) applies to the concentration change in the user's body, the user is a good 24 minutes above the desired therapeutic effect concentration, namely from minute 18 to minute 42. Said user would not perform an actuation until minute 42 at time tB_{2_M}, which, after a predetermination, finally leads at the application point of time tA_{2_M} to a re-dispensing of a (second) application dose D_{2_M} requested by them.

Case 2: If the population pharmacokinetics curve PK30_{D1_+SD} (mean plus one standard deviation) applies to the concentration change in the user's body, the user would be 21 minutes above the desired therapeutic effect concentration, namely from minute 18 to minute 39. Said user would not make an actuation until minute 39 at the point of time tB_{2_+SD}, which after a predetermination finally leads at the application point of time tA_{2_+SD} to a re-dispensing of a (second) application dose D_{2_+SD} requested by them.

Case 3: If the population pharmacokinetics curve PK30_{D1_-SD} (mean minus one standard deviation) applies to the concentration change in the user's body, the user would be a good 27 minutes above the desired therapeutic effect concentration, namely from minute 18 to minute 45. Said user would not dispense an actuation until minute 45 at point of time tB_{2_-SD}, which, after a predetermination, would eventually lead at the application point of time tA_{2_-SD} to a re-dispensing of a (second) application dose D_{2_-SD} requested by them.

On the basis of the actuation points of time tB_{2_+SD}, tB_{2_M}, or respectively tB_{2_-SD}, the control device 9 thus recognizes whether the user's individual PK curve is now the PK curve PK30_{D1_+SD}, the PK curve PK30_{D1_M}, or the PK curve PK30_{D1_-SD} (or one of the countless further PK curves which the control device 9 may also consider). If the requesting time is at tA_{2_+SD}, the PK curve PK30_{D1_+SD} applies. If the requesting time is at tA_{2_M}, the PK curve PK30_{D1_M} applies. If the requesting time is at tA_{2_-SD}, the PK curve PK30_{D1_-SD} applies.

If an initial direct association between a temporal course and the considered user is not possible, the control device 9, nevertheless, approaches the target using a process of elimination and categorizes/determines for the specific user on the basis of their actuation behavior which temporal concentration course or which curve reflects them best. After the “titration phase” (which is a determination phase or finding phase), which is shown in FIG. 5b and may possibly extend beyond the times shown in FIG. 5b, a categorization or classification of the user may take place. Subsequently, the amount of the following application doses may be determined and/or an individual, dose-based or concentration-based treatment plan can be set up in a time-controlled manner.

If one follows the above procedure, then it is possible, by postulating one or any number of PK curves as being possible, individual PK curves of the user, a priori or as a plurality of hypotheses (such as: the PK curve PK30_{D1_+SD} applies to the user, or the PK curve PK30_{D1_M} applies to the user, or the PK curve PK30_{D1_-SD} applies to the user), to generally check the correctness of the applicable or raised hypotheses already following a first initial dose or application dose D₀, D₁, D₂, etc., or a next application dose Dₙ. This results in an individual pharmacokinetic model for the user, which may be used as a basis for future application doses. In the example of FIG. 5b, the PK curve PK30_{D1_+SD} is defined at the actuation point of time tB_{2_+SD} as the PK curve PK_{Ind} and used as the basis for the necessary calculations in future predeterminations of application doses.

A check whether the individual pharmacokinetic model determined in this way does actually apply, may optionally be carried out at any time in the course of further treatment and in connection with further application doses Dₙ, Dₙ₊₁, etc.

FIG. 6 shows a nasal applicator 100 of a further exemplary embodiment. The nasal applicator 100 is shown in FIG. 6 in a sectional illustration or in a—partially indicated—partial section.

Using the requesting device 1, the user may forward a next application dose Dₙ of the substance S.

In the following, there will discussed those components which have not already been explained with reference to FIG. 1.

Thus, the nasal applicator 100 comprises a dispenser 3 which conducts a dosing of the application dose to be dispensed, i.e. determines the quantity. The dispenser 3 is exemplarily designed here as a loading device 15, which defines the amount of the application dose to be delivered by determining how large this amount will be, respectively. In order to segregate the desired application dose, the loading device 15 may optionally be pulled back further or less far—seen from a nasal attachment 4—along the double arrow, for instance automatically or by hand.

An application device 17 for—here: actively—applying the respectively requested application dose via the connection site 4a or the nasal attachment 4 is exemplarily provided in FIG. 6 as a combination consisting of, or having, a motor, here e.g. an electric motor 19, and a spindle 21.

Electric motors, as referred to herein, may be, for example, brushless or have a brush. They may be designed, for example, as internal rotor motors, external rotor motors and/or slotless motors. An encoder may be integrated, such an encoder may be arranged externally, the same applies to a controller of the motor.

The motor, in some embodiments, may be a stepper motor such as a hybrid stepper motor, a flat motor, a hollow-shaft motor, a servo motor, an asynchronous motor, a synchronous motor, or a traveling wave motor, or the like.

In several embodiments, the motor may be controlled using open control and/or closed-loop control, and/or using an encoder and/or a controller. Encoders may be, for example, optical transmitted light encoders or reflective encoders, magnetic encoders or the like.

The motor may be or is controlled via the current consumption. If for example the motor approaches or moves to an end position (up or down), the current consumption of the motor increases e.g. from 0.5 A to 1 A. This means that an end position or stop has been reached. This measurement value may, therefore, also be used to control the motor, i.e. to signal (to it) when e.g. it should switch off.

In order to control the nasal applicator or its motor, a displacement sensor, a flow sensor, limit switch (microswitch) may optionally be used. Additionally or alternatively, a timed control (e.g. energization for a predetermined time period) is also encompassed by the present invention.

The motor, or the spindle combination, may in certain embodiments comprise a brake, e.g. a magnetic brake, eddy current, spring pressure brake, a limit switch at top and/or bottom dead center or limit point of the stroke, etc.

The motor or motor-spindle combination may alternatively or additionally include a damper to eliminate or improve, e.g., resonance and noise problems.

The nasal applicator 100 of FIG. 6 optionally comprises a dose chamber 13 distinguishable from the substance reservoir R. It serves to receive the application dose of the substance S stored in the substance reservoir R, for which in particular any embodiment disclosed herein may be provided, in order to subsequently dispense it.

In order to be able to dispense the application dose, the dose chamber 13 must first be filled with it. In the embodiment of FIG. 6, this task is performed by the loading device 15.

The dose chamber 13 may be actuated to execute a sequence of a suction stroke and a dispensing stroke, as described below.

Deaeration of the system works identically, except that air is a compressible medium and substance S is a non-compressible medium. When or by deaerating, the system will travel the full stroke path and may optionally aim for different parameter values for speed, force, pressure and/or acceleration of or for deaeration as after deaeration, and in particular for the delivery of the substance S.

When the loading device 15 and/or the application device 17 is moved away from the upper dead center/end point/beginning point/initial point (on the side of the one-way valve 23b) with respect to the orientation in FIG. 6, a negative pressure is created in the dose chamber 13. This negative pressure allows substance S to flow out of the substance reservoir R into the dose chamber 13. The plug 17a in the substance reservoir R is also trailed by this negative pressure and thus a collapsible container is created (suction stroke). In this, the one-way valve 23b closes, and the one-way valve 23a opens. This may be done mechanically, pneumatically, hydraulically, electrically, etc.

As an alternative to controlling the valves 23a, 23b by negative pressure, the valves 23a, 23b may be controlled electrically. For this purpose, the position of the loading device 15 is reverted to.

The valves 23a, 23b may also be designed or operated in any combination of the aforementioned modes of operation.

In order to apply the substance S (dispensing stroke) out of the dose chamber 13, the direction of the loading device 15 is reversed, and it thereby becomes the application device 17, which travels with a predetermined speed, force, pressure, acceleration in the direction towards the one-way valve 23b. When the path is reversed, a positive pressure is created in the dose chamber 13, which closes the one-way valve 23a and opens the one-way valve 23b. This allows the substance S to be forced through the nasal attachment 4 via the connection site 4a. The valve control may be as described above.

Optionally, different values for speed, force, pressure and/or acceleration are applied or aimed for in the suction stroke than in the dispensing stroke.

The dose chamber 13 may be manufactured with high precision to provoke minimal variability in the delivery quantity, which may also apply to the loading device 15 and the application device 17.

This can be achieved by using a plastic with low shrinkage or low water absorption. Alternatively or additionally, e.g. in FIG. 6, a sleeve may be pressed-in which preferably has very close tolerances in diameter. This allows better control of the application volume. The application volume is given by the formula:

volume=area*stroke. The area is that of a circle A=d²*π/4.

Plastics with low shrinkage or low water absorption can be called “dimensionally stable plastics”.

Dimensional stability refers to the ability of polymers to maintain their size under various environmental conditions. A dimensionally stable plastic, therefore, tends less to moisture absorption and displays less thermal expansion.

Dimensional stable plastics include polymers such as PEEK, PPS, PSU, PPSU, PEI and PET.

Moisture absorption may lead not only to a change in size, but also to a change in material properties. This may have an effect on, among others, the mechanical strength and/or electrical properties, such as electrical conductivity and the dielectric loss factor.

A polymer without water absorption is, for example, PTFE. The following polymers are plastics with very low water absorption: PEEK, PPS, PSU, PPSU, PEI, PVDF, PET, PPE, PP and PE. Low water absorption is also recorded for POM, PA12, PC and ABS.

The dose chamber 13, the application device 17, and/or the loading device 15 may be self-sealing and/or comprise a seal such as a sealing ring 16, piston rings, O-rings, sealing lips, etc.

The dose chamber 13, and/or any elements that come into contact with the substance such as the application device 17, the loading device 15 and/or others, may be coated. A possible coating may be e.g. parylene, and/or may be of inert materials such as glass, and/or may be of technical plastics such as cycloolefin copolymers (COC), cyclo-olefin polymers (COP), etc.

Here, as in any other embodiments, the dose chamber 13, the application device 17, and/or the loading device 15 may consist of or comprise materials that do not require lubrication, e.g., POM homopolymer, Teflon, etc. Lubricants, like e.g. silicone oil, may also be used, or coatings with which friction is reduced. In some embodiments, additives may also be added to the plastics to improve sliding properties such as, e.g., CORDULEN, montan wax, high molecular weight polyethylene wax (PE-LD, PE-HD), fluoroelastomers, etc.

In certain embodiments, the dose chamber 13, the application device 17, and/or the loading device 15 may consist of or comprise other materials such as plastic.

As can be seen from the foregoing explanations, the dose chamber 13 is variable in that the loading device 15 separates sometimes more and sometimes less amount of substance S from the substance reservoir R, wherein the space in which the thus separated application dose is present before it is delivered may be defined as the dose chamber 13.

The substance reservoir R may be or will be clamped, rotated, inserted, fixed in a guide or may be or will be guided in the housing.

The substance reservoir R may be connected to the loading device 15, the application device 17, the nasal attachment 4, the dispenser 3, or the connection site 4a. The connection may be established by a flexible or rigid line 22. The line 22 may be stationary, or may be movable with the application device 17 and/or the loading device 15.

The substance reservoir R may be connected to the dose chamber 13 by, e.g., a Luer lock, pin, needle, spike with or without a vent, etc.

To establish a fluid connection between the dose chamber 13 and the substance reservoir R, e.g. a needle 38 may be provided, which pierces e.g. a septum 39 of the vial.

A filter, e.g. a (e.g. 22 μm) particle filter or a porous membrane (e.g. a Porex®—filter), may be interposed. In some embodiments, this filter may be disposed at the outlet of the dose chamber 13, and a second filter may additionally be provided there.

As will become clear from the following elaborations, the loading device 15 may be identical to the application device 17, but it may also be provided separately therefrom.

Although the application device 17 and/or the loading device 15 may have an energy storage for mechanical energy or spring energy and/or hydraulic and pneumatic energy as an energy application mechanism, this is not the case in the embodiment of FIG. 6.

The electrical energy storage 20 may be integrated in the application device 17 and/or the loading device 15 or connected to them by an external connection.

The integrated energy storage 20 may be charged for a release and may be charged after each application by a charging device, e.g. a Qi charging pad, or in a cradle by a PIN connection.

The electrical energy storage device 20 may be a capacitor or the like.

Power may be supplied via power cable, power supply, etc., or via Ethernet (LAN), Power over Ethernet (PoE). The latter refers to a method of supplying power to network-enabled devices via the eight-wire Ethernet cable. Such a supply may be provided herein. In some embodiments, solar cells or mechanisms for energy harvesting may alternatively be provided for this purpose.

In FIG. 6, the application device 17 and/or the loading device 15 are designed as an electric drive, herein with electric motor 19 having a spindle 21, which are coupled.

In other embodiments, this coupling may also take place via a transmission or be designed with a transmission.

With reference to FIG. 6, it should be noted that in several embodiments, such as that of FIG. 6, the application device 17 and the loading device 15 may be implemented or realized by a common mechanism, wherein the mechanism, for example, when it travels in a first direction (for example away from the connection site 4a), fills the dose chamber 13 as the loading device 15, and when it travels in a second direction, which may be opposite to the first (for example towards the connection site 4a), discharges the contents of the dose chamber 13 as the application device 17, for example, via the nasal piece. A counter-rotation of spindle 21 and/or motor 19 during suction on the one hand and application on the other hand may be provided, as has already been described elsewhere herein. Alternatively, rotation in the same direction is possible for both filling and emptying the dose chamber 13, as explained above, for example, with reference to the crankshaft gear. Likewise, filling and emptying are possible with mutual rotation when the crankshaft gear is used, as described above.

The application device 17 and/or the loading device 15 are releasably or non-releasably connected to the spindle 21 and/or the motor 19.

The substance reservoir R, the application device 17 and/or the loading device 15 may be designed as disposables.

The application device 17, the loading device 15 may be designed as a reusable product.

Both optionally provided housing shells may be designed as disposables.

The electronic control device 9, motor 19, spindle 21, and/or other components may be reusable.

The nasal applicator 100 of FIG. 6 further comprises an optional mechanism for changing the capacity volume of the dose chamber 13. By or with this mechanism, the capacity volume of the dose chamber 13 may be varied in order to have or receive a partial amount of the substance S, making it possible to apply application doses of different sizes.

In some embodiments, this mechanism is part of the dispenser 3, but in others such as the present one, it is not.

The electronic control device 9, see FIG. 1, if provided, may be programmed to move and/or cause the application device 17 to apply at a variety of speeds, forces, pressures, accelerations, flow rates.

A motor-driven, nasal device may be used to solve the problem of each user applying a different speed, force, pressure, acceleration when releasing manually through pressing. It is not guaranteed that each user will generate the same speed, e.g. release speed or ideal release speed. Since the release speed has the greatest influence on the droplet size distribution, the spray pattern and the plume geometry, a release that is always identical between users is of particular advantage, since this always ensures a consistent spray pattern, in particular the droplet size distribution, the spray pattern and the plume geometry and application volume, compared to a manual release.

In this way, it can be advantageously ensured that when the next application dose is applied, the stroke path required for this, i.e. the complete stroke distance, is always covered. If e.g. for a 150 μL dose, a distance of 5 mm has to be covered, then it can be ensured as described herein that not only e.g. 4 mm are covered, which could be possible with a manual solution based on the pressure strength or pressure depth, but, due to the options described herein, always the complete required stroke path is covered.

Additionally or alternatively, the electronic control device 9 may be programmed in order to act on the mechanism for changing the capacity volume of the dose chamber 13. This may also be done manually and the release may still be automatic.

The droplet size distribution, the spray pattern, the plume geometry may preferably be adjusted, and optionally adjusted differently for each application amount.

Depending on the agent and its properties, such as, e.g., viscosity, surface tension, etc., a different speed, a different force, a different pressure, a different acceleration, a different flow rate may be required. The device may, in some embodiments, run at a separate speed, force, acceleration, flow rate, or pressure for each dose amount. This is set or may be set, for example, in advance.

In certain embodiments, a sensor may detect the position or release position, e.g. vertical or horizontal (and everything in between), and then adjust the speed, force, pressure, acceleration to that position, respectively.

In the embodiment of FIG. 6, the loading device 15 for loading the dose chamber 13 with the application dose of substance S, comprises at least one first one-way valve 23a or check valve and/or the dose chamber 13 is (de)limited by at least one first one-way valve 23a or check valve. This valve may be arranged, e.g., in a piston itself or at any position, e.g. at any position in the inlet line from the substance reservoir R to the dose chamber 13.

In the embodiment of FIG. 6, the loading device 15 comprises at least one second one-way valve 23b or check valve.

Alternatively, e.g., the first valve 23a is designed as a switchable three-way valve and/or designed differently. Thus, the first valve 23a may also perform the function of two valves as a singular element.

In this, the dose chamber 13 is exemplarily limited by at least the second one-way valve 23b or check valve.

Optionally, the second check valve 23b comprises relative to the dose chamber 13 an opening direction opposite to that of the first check valve 23a.

Optionally, the function of the first one-way valve 23a and the function of the second one-way valve 23b may be combined into a common component, like e.g. a valve element.

The dose chamber 13 may optionally be in communication with, or brought into communication with, the atmosphere via a deaeration valve, not shown herein. It may be provided to use such a deaeration valve to operate the initial deaeration of the system and/or to eliminate air bubbles at a later time. A filter may be provided at a suitable position.

The device optionally comprises two housing parts, namely an upper housing part 2b and a lower housing part 2c, which are separated by a housing splitline 2d in FIG. 6.

In the schematic representation of FIG. 6, the housing upper part 2b contains the substance reservoir R, the dose chamber 13, the application device 17 and/or the loading device 15. Valves, filters, deaeration, the connection site 4a for the nasal attachment 4, etc. may also be provided in the housing upper part 2b.

The housing lower part 2c comprises the electronic control device 9, the motor 19, the spindle 21, the optional encoder, the controller, the brake, the damper, the energy storage 20, the requesting device 1, etc.

Components mentioned above are arranged only exemplarily as shown in FIG. 6. They may be arranged in any combination.

One or both of the housing shells or housing parts 2b, 2c may be disposables. The nasal applicator 100 may be present completely assembled, sterilized, packaged or may be shipped or delivered completely assembled, sterilized, packaged. Alternatively, it is first completed (or assembled) on site, at the patient's bedside, by the patient, by the physician, etc. For example, the substance reservoir R may still have to be inserted into the housing upper shell and the latter then assembled with the housing lower shell.

The electronic control device 9, the motor 19, the spindle 21, the optional encoder, the controller, the brake, the damper, the energy storage 20 and/or the requesting device 1, may, in any combination with each other, form a unit. They can be, or are, inserted into a housing lower part 2c, which then forms the housing 2 together with the housing upper part 2b. Optionally, upon disassembly, the housing shells (upper and lower parts) 2b, 2c could be opened and disposed of. The electronics may be removed, without being touched, from the lower shell or housing lower part 2c.

The housing parts 2b, 2c, alone or together, may for example also be intentionally destroyed after completion of the user's treatment in order to prevent further use of the nasal applicator. To this end, it may be provided to break off e.g. an optional snap hook or to irreversibly damage, break off, displace, etc. another form-fit connection. For example, one deforms the snap hook, displaces a connecting member or lock, etc.

The nasal applicator 100 may be automatically and/or manually emptied after treatment and discarded as a whole. Alternatively, it may be disassembled into parts and only partially discarded or reprocessed or reused, in particular, for example, the electronic control device 9, the motor 19, the spindle 21, etc. are suitable for this purpose.

The nasal applicator 100 may comprise a lid which may be opened for inserting the substance reservoir R and/or the electronic control device 9.

A sealing ring 16 may be provided to ensure tightness between the loading device 15 and/or application device 17, on the one hand, and the surrounding sleeve or shaft, on the other hand.

A flow sensor 11a may be provided. It may be part of or constitute or represent the detection device.

An optional plug 17a is provided to limit the volume of the substance reservoir R, it may be arranged in a shiftable manner.

An optional spring may be provided in this, or any other, embodiment, which presses directly or indirectly against the plug 17a to move it, upon release of the nasal applicator 100, out of a position it may have assumed over an extended time period due to storage reasons.

The valve 23a shown in FIG. 6, may in any embodiment be disposed optionally, at, on, and/or within the application device 17 and/or the loading device 15 or in close proximity thereto.

Optionally, the line 22 through which the substance S is delivered to the dose chamber 13 may in arbitrary embodiments extend through the loading device 15 and/or the application device 17.

Optionally, the loading device 15 and/or the application device 17, may in arbitrary embodiments have a connection for the line 22.

Optionally, the loading device 15 and/or the application device 17, may in arbitrary embodiments be hollow or unobstructed.

Optionally, the line 22, may in arbitrary embodiments move together with the loading device 15 and/or together with the application device 17. In some other embodiments, the line 22 does not move with the loading device 15 and/or the application device 17.

Optionally, the substance reservoir R may in arbitrary embodiments be installed to be rotated 180 degrees relative to the embodiment of FIG. 6.

Optionally, all components may be positioned as desired in arbitrary embodiments to avoid blind installation, i.e. installation without visibility, reduce the risk of injury from needle 38, and that the substance reservoir R, which may comprise the septum 39 or other connection, can be more easily installed.

### LIST OF REFERENCE NUMERALS

100 nasal applicator
101 peripheral device
1 requesting device
2 housing
2b upper housing part
2c lower housing part
2d housing splitline
3 dispenser
4 nasal attachment
4a connection site
locking device
7 dose-detection device
8 feedback device
9 electronic control device
11 detection device
11a flow sensor
12 display
13 dose chamber
15 loading device
16 seal or sealing ring
17 application device
17a plug
19 electric motor
20 energy storage
21 spindle
22 line
23a first one-way valve, check valve
23b second one-way valve, check valve
38 needle
39 septum
C_{ED1} target concentration, concentration required for the desired effect/impact at actuation point of time tB₁
C_{ED2} target concentration, concentration required for the desired effect/impact at actuation point of time tB₂
C_{ED3} target concentration, concentration required for the desired effect/impact at actuation point of time tB₃
C_{ED1_+SD} first set target concentration, concentration required for the desired effect/impact on the PK curve PK50_{D0_+SD} at actuation point of time tB₁
C_{ED1_M} first set target concentration, concentration required for the desired effect/impact on the PK curve PK50_{D0_M} at actuation point of time tB₁
C_{ED1_-SD} first set target concentration, concentration required for the desired effect/impact on the PK curve PK50_{D0_-SD} at actuation point of time tB₁
C_{ED2_+SD} second set or first estimated target concentration, concentration required for the desired effect/impact on the PK curve PK30_{D1_+SD} at actuation point of time tB₂
C_{ED2_M} second set or first estimated target concentration, concentration required for the desired effect/impact on the PK curve PK30_{D1_M} at actuation point of time tB₂
C_{ED2_-SD} second set or first estimated target concentration, concentration required for the desired effect/impact on the PK curve PK30_{D1_-SD} at actuation point of time tB₂
C′_{ED1} first target concentration determined before the dispensing point of time tB₁, concentration required for the desired effect/impact on the PK curve PK50_{D0}
C′_{ED1_+SD} first target concentration determined before the dispensing point of time tB₁, concentration required for the desired effect/impact on the PK curve PK50_{D0_+SD}
C′_{ED1_M} first target concentration determined before the dispensing point of time tB₁, concentration required for the desired effect/impact on the PK curve PK50_{D0_M}
C′_{ED1_-SD} first target concentration determined before the dispensing point of time tB₁, concentration required for the desired effect/impact on the PK curve PK50_{D0_-SD}
C(t) concentration at point of time t
Cₙₘₐₓ concentration maximum
C₁ₘₐₓ first maximum on the PK curve PK50_{D0}
C₂ₘₐₓ second maximum on the PK curve PK25_{D1}
C₃ₘₐₓ third maximum on the PK curve PK25_{D2}
C_{1max_+SD} first concentration maximum on the PK curve PK50_{D0_+SD}
C_{1max_M} first concentration maximum on the PK curve PK50_{D0_M}
C_{1max_-SD} first concentration maximum on the PK curve PK50_{D0_-SD}
C_{2max_+SD} second concentration maximum on the PK curve PK30_{D1_+SD}
C_{2max_M} second concentration maximum on the PK curve PK30_{D1_M}
C_{2max_-SD} second concentration maximum on the PK curve PK30_{D1_-SD}
D dose
D₀ initial dose
D₁ first application dose, preceding application dose
D₂ second application dose, subsequent application dose
D₃ third application dose
Dₙ₋₁ the application dose preceding the next application dose
Dₙ next application dose
Dₙ₊₁ the application dose following the next application dose or the one after the next dose
M data storage
PK25 PK curve for 25 μg fentanyl single dose
PK50 PK curve for 50 μg fentanyl single dose
PK25_{Dn} PK curve for 25 μg fentanyl multiple dose
PK50_{Dn} PK curve for 50 μg fentanyl multiple dose
PK50_{+SD} PK curve for 50 μg fentanyl single dose, mean value over all considered individuals of the collective plus one standard deviation
PK50_{M} PK curve for 50 μg fentanyl single dose, mean value over all considered individuals of the collective
PK50_{-SD} PK curve for 50 μg fentanyl single dose, mean value over all considered individuals of the collective minus one standard deviation
PK50_{Dn_+SD} PK curve for 50 μg fentanyl multiple dose, mean value over all considered individuals of the collective plus one standard deviation
PK50_{Dn_M} PK curve for 50 μg fentanyl multiple dose, mean value over all considered individuals of the collective
PK50_{Dn_-SD} PK curve for 50 μg fentanyl multiple dose, mean value over all considered individuals of the collective minus one standard deviation
PK30_{Dn_+SD} PK curve for 30 μg fentanyl multiple dose, mean value over all considered individuals of the collective plus one standard deviation
PK30_{Dn_M} PK curve for 30 μg fentanyl multiple dose, mean value over all considered individuals of the collective
PK30_{Dn_-SD} PK curve for 30 μg fentanyl multiple dose, mean value over all individuals considered in the collective minus one standard deviation
PK_{Ind} individual PK curve
R substance reservoir
S medical substance
T_vainₙ locking periods
T_vain_{n_S} locking period begin
T_vain_{n_E} locking period end
tA₀ initial or beginning time; dispensing point of time of the initial dose; initial or beginning dose
tA₁, . . . , tAₙ, tAₙ₊₁ dispensing point of time 1 to n+1
tA_{2_+SD} mean value of dispensing point of time over all considered individuals of the collective plus one standard deviation
tA_{2_M} mean value of dispensing point of time over all considered individuals of the collective
tA_{2_-SD} mean value of dispensing point of time over all considered individuals of the collective minus one standard deviation
tB₁, . . . , tBₙ, tBₙ₊₁ actuation point of time 1 to n+1 tB_{2_+SD}, tB_{2_M}, tB_{2_-SD} calculated target concentration time
tB_{1_v1}, tB_{2_v1} futile actuation points of time during the locking period T_vain₁
tB_{1_v2}, tB_{2_v2} futile actuation points of time during the locking period T_vain₂
tB_{m_vn} futile actuation points of time during the locking period T_vainₙ
tF_{n_1} first feedback points of time
tF_{n_2}, tF_{n_3} feedback points of time
tF_{n_4} last feedback point of time
tV₁, . . . , tVₙ, tVₙ₊₁ predetermination point of time 1 to n+1

1. A nasal applicator (100) for the nasal administering of at least one medical substance (S), in particular an analgesic, said nasal applicator (100) comprising a housing (2) which respectively comprises, or is connected to,
a substance reservoir (R) for comprising a quantity of the substance (S);
a requesting device (1) to be actuated by the user with the aim of requesting a next application dose (Dₙ) of the substance (S);
a dispenser (3) for dispensing application doses (D₀, D₁, D₂, . . . , Dₙ₋₁, Dₙ, Dₙ₊₁, . . . ) upon actuation of the requesting device (1) at a respective dispensing point of time (tA₀, tA₁, tA₂, . . . , tAₙ₋₁, tAₙ, tAₙ₊₁, . . . );
a connection site (4a) for a nasal attachment (4), or a nasal attachment (4) or a nasal piece; and
an application device (17) for applying an application dose of the substance (S) through the connection site (4a) or the nasal attachment (4) or the nasal piece and/or out of the nasal applicator (100).

2. The nasal applicator (100) according to claim 1, further comprising
a dose chamber (13) for temporarily receiving the application dose of the substance (S) held in the substance reservoir (R); and
a loading device (15) for loading the dose chamber (13) with the application dose of the substance (S) from the substance reservoir (R).

3. The nasal applicator (100) according to claim 2, wherein the application device (17) is arranged for applying the application dose of the substance (S) present in the dose chamber (13) through the connection site (4a), the nasal attachment (4) or the nasal piece and/or out of the nasal applicator (100).

4. The nasal applicator (100) according to claim 2, wherein the application device (17) and/or the loading device (15) comprises an energy storage for mechanical energy or spring energy.

5. The nasal applicator (100) according to claim 1, wherein the application device (17) and/or the loading device (15) comprises a motor, preferably an electric motor (19).

6. The nasal applicator (100) according to claim 1, wherein the application device (17) and/or the loading device (15) comprises a spindle (21).

7. The nasal applicator (100) according to claim 6, wherein the motor is connected to the spindle (21).

8. The nasal applicator (100) according to claim 1, further comprising
a mechanism for changing the capacity volume of the dose chamber (13) for the application dose of the substance (S).

9. The nasal applicator (100) according to claim 1, further comprising
a second fluid chamber comprising a desiccant or a substance neutralizing agent.

10. The nasal applicator (100) according to claim 1, wherein the application device (17) and the loading device (15) comprise or consist of identical components.

11. The nasal applicator (100) according to claim 1, further comprising
an electronic control device (9).

12. The nasal applicator (100) according to claim 11, wherein the electronic control device (9) is programmed to make the application device (17) move and/or apply at a plurality of speeds.

13. The nasal applicator (100) according to claim 11, wherein the electronic control device (9) is programmed to act on the mechanism for changing the capacity volume of the dose chamber (13).

14. The nasal applicator (100) according to claim 2, wherein the loading device (15) for loading the dose chamber (13) with the application dose of the substance (S) comprises at least one first one-way valve (23a) or check valve, and/or wherein the dose chamber (13) is limited by at least one first one-way valve (23a) or check valve.

15. The nasal applicator (100) according to claim 2, wherein the loading device (15) for loading the dose chamber (13) with the application dose of the substance (S) comprises at least one second one-way valve (23b) or check valve, and/or wherein the dose chamber (13) is limited by at least one second one-way valve (23b) or check valve, wherein the second check valve (23b) preferably comprises an opening direction being, with respect to the dose chamber (13), opposite to that of the first check valve (23a).

16. The nasal applicator (100) according to claim 1, further comprising:
a locking device (5) for temporarily locking the dispenser (3) during a re-begin of locking period (T_vain₁, T_vain₂, . . . , T_vainₙ₋₁, T_vainₙ, T_vainₙ₊₁, . . . ) that begins again at or after the respective dispensing point of time (tA₀, tA₁, tA₂, . . . , tAₙ₋₁, tAₙ, tAₙ₊₁, . . . ) of the dispensed application dose (D₀, D₁, D₂, . . . , Dₙ₋₁, Dₙ, Dₙ₊₁, . . . ), the locking period (T_vain₁, T_vain₂, . . . , T_vainₙ₋₁, T_vainₙ, T_vainₙ₊₁, . . . ) having a locking period begin (T_vain_{1_S}, T_vain_{2_S}, . . . , T_vain_{n-1_S}, T_vain_{n_S}, T_vain_{n+1_S}, . . . ) and a locking period end (T_vain_{1_E}, T_vain_{2_E}, . . . , T_vain_{n-1_E}, T_vain_{n_E}, T_vain_{n+1_E}, . . . )

17. The nasal applicator (100) according to claim 1, further comprising:
a dose-detection device (7) for detecting the amount of the application dose (D₀, D₁, D₂, . . . , Dₙ₋₁, Dₙ, Dₙ₊₁, . . . ) respectively dispensed by the dispenser (3) at the dispensing point of time (tA₀, tA₁, tA₂, . . . , tAₙ₋₁, tAₙ, tAₙ₊₁, . . . ).

18. The nasal applicator (100) according to claim 1, further comprising:
a data storage (M) for storing at least the dispensing points of time (tA₀, tA₁, tA₂, . . . , tAₙ₋₁) of one or more already dispensed application doses (D₀, D₁, D₂, . . . , Dₙ₋₁) and/or the amounts of these application doses (D₀, D₁, D₂, . . . , Dₙ₋₁).

19. The nasal applicator (100) according to claim 1, further comprising:
a detection device (11) at least for detecting an activation behavior of the user at an actuation point of time (tBₙ) relating to the requesting device (1) with the aim of applying a next or further application dose (Dₙ).

20. The nasal applicator (100) according to claim 11, wherein the electronic control device (9) is further programmed to
read data stored in the data storage (M);
evaluate the user's actuation behavior detected by the detection device (11);
predetermine, at a predetermination point of time (tVₙ), the amount of the next or the further application dose (Dₙ) which may be delivered to the user at a next dispensing point of time (tAₙ) in the event that the user actuates the requesting device (1) outside a locking period (T_vainₙ), wherein the predetermination point of time (tVₙ) lies at or after the actuation point of time (tBₙ), and wherein the predetermination takes place considering the data read from the data storage (M), wherein this data encompasses at least a) the dispensing point of time (tA₀, tA₁, tA₂, . . . , tAₙ₋₁) of one or several of the already dispensed application doses (D₀, D₁, D₂, . . . , Dₙ₋₁), or the elapsing time period between the one or the several dispensing point of time(s) (tA₀, tA₁, tA₂, . . . , tAₙ₋₁) of the already dispensed application doses (D₀, D₁, D₂, . . . , Dₙ₋₁) and the dispensing point of time (tAₙ) lying after the next actuation point of time (tBₙ), on the one hand and/or b) the amount of one or of several of the already dispensed application doses (D₀, D₁, D₂, . . . , Dₙ₋₁) on the other hand.

21. The nasal applicator (100) according to claim 20, wherein
the data storage (M) further encompasses pharmacological, pharmacodynamic, pharmacometric and/or pharmacokinetic data concerning the substance (S) or other data associated with the substance, wherein
the electronic control device (9) is further programmed for additional reading of data stored in the data storage (M) concerning the substance (S) or data associated with the substance (S), and wherein
predetermining the next application dose (Dₙ) of the substance (S) takes place with additional consideration of the additionally read data.

22. The nasal applicator (100) according to claim 20, wherein said electronic control device (9) is further programmed to cause or initiate the further steps of:
dispensing the next application dose (Dₙ) in the predetermined amount at the corresponding dispensing point of time (tAₙ) by the dispenser (3); and subsequently
storing the amount, and/or a concentration resulting thereof, of the application dose of the dispensed next application dose (Dₙ) in the data storage (M) and causing the locking device (5) to temporarily lock the dispenser (3) for a locking period (T_vainₙ₊₁), beginning from or after the dispensing point of time (tAₙ) of this next application dose (Dₙ), the locking having a locking period begin (T_vain_{n+1_S}) and locking period end (T_vain_{n+1_E}) and/or storing the dispensing point of time (tAₙ) of the dispensed, next application dose (Dₙ) in the data storage (M).

23. The nasal applicator (100) according to claim 22, wherein the electronic control device (9) is further programmed to carry out or prompt or intitiate the sequence of the further steps of claim 22 also in connection with one or several of the deliveries of further application doses (Dₙ₊₁) subsequent to the next application dose (Dₙ).

24. The nasal applicator (100) according to claim 23, wherein the electronic control device (9) is further programmed to read from the data storage (M) data encompassing e.g. PK curves (PK25, PK50) and/or models, and which map a temporal course between concentration (C(t)) in the body, in particular blood, of the patient, and to calculate a next maximum (C₁ₘₐₓ, C₂ₘₐₓ, Cₙₘₐₓ) of the concentration (C(t)) based thereon.

25. The nasal applicator (100) according to claim 24, wherein the electronic control device (9) is further programmed, upon predetermining the amount of the next application dose (Dₙ) based on data stored in the data storage (M), in particular PK curves (PK25, PK50) or models, and on a therapeutic maximum stored in the data storage (M), to determine the amount of the next application dose (Dₙ) such that the next maximum (C₁ₘₐₓ, C₂ₘₐₓ, Cₙₘₐₓ) will not exceed the specified maximum.

26. The nasal applicator (100) according to claim 20, wherein
evaluating the detected actuation behavior of the user encompasses determining the actuation point of time (tBₙ) for requesting the next application dose (Dₙ), or the time span lying between this actuation point of time (tBₙ) and the locking period end (T_vain_{n_E}) of the last expired locking period (T_vainₙ); wherein
predetermining encompasses considering the determined next actuation point of time (tBₙ) or the determined time span.

27. The nasal applicator (100) according to claim 9, further comprising
a feedback device (8) to be actuated by the user;
wherein the detection device (11) is programmed for detecting actuation of the feedback device (8) at least at one, preferably first, feedback point of time (tF_{n_1});
wherein the control device (9) is programmed for determining the concentration of the substance (S) present in the body or in the blood of the user at the at least one feedback point of time (tF_{n_1}), and for setting a target concentration (C_{ED}) or its maximum value to a value respectively below the determined concentration.

28. The nasal applicator (100) according to claim 27,
wherein the detection device (11) is further programmed for detecting at least one actuation of the requesting device (1) made by the user at actuation points of time (tB₁, tB₂, . . . , tBₙ₋₁, tBₙ, tBₙ₊₁, . . . ) after the locking period (T_vain₁, T_vain₂, . . . , T_vainₙ₋₁, T_vainₙ, T_vainₙ₊₁, . . . ) and/or for detecting a, preferably last, feedback point of time (tF_{n_4}) after from which no further actuations of the feedback device (8) by the user have been detected;
wherein the control device (9) is further programmed
for determining both a concentration (C(t)) in the body, in particular in the blood, associated with the, preferably first, feedback point of time (tF_{n_1}) as well as a concentration (C(t)) in the body, in particular in the blood, associated with the, preferably first, actuation point of time (tB₁, tB₂, . . . , tBₙ₋₁, tBₙ, tBₙ₊₁, . . . ) or with the last feedback point of time (tF_{n_4}), respectively;
for determining a temporal concentration course, or a concentration course over time, from a group of temporal concentration courses, in particular models or PK curves (PK50_{+SD}, PK50_{M} und PK50_{-SD}), associated with the same dose, from a plurality of temporal concentration courses, in particular PK models or PK curves, associated with the same dose and stored in the data storage (M), while considering both the, preferably first, feedback point of time (tF_{n_1}) and a concentration associated with the, preferably first, actuation point of time (tB₁, tB₂, . . . , tBₙ₋₁, tBₙ, tBₙ₊₁, . . . ) or with the last feedback point of time (tF_{n_4}); and
for considering the temporal individual concentration course determined in this way, in particular the determined individual PK curve (PK_{Ind}), upon predetermining the amount of one or several of the subsequent application doses (D₃, Dₙ, Dₙ₊₁, . . . ).

29. The nasal applicator (100) according to claim 27, wherein the control device (9) is further programmed
for considering, upon predetermining the amount of the next application dose (Dₙ), the specified target concentration (C_{ED}) or its maximum value and/or the determined temporal individual concentration course, in particular the determined individual PK curve (PK_{Ind}).

30. The nasal applicator (100) according to claim 9, wherein the control device (9) is further programmed
for determining, after the dispensing of an initial dose (D₀) which was dispensed at a dispensing point of time (tA₀) at or for a first actuation point of time (tB₁) which lies after the dispensing point of time (tA₀) of the initial dose (D₀), respectively, a concentration (C_{ED1_+SD}, C_{ED1_M}, C_{ED1_-SD}) associated with the first actuation point of time (tB₁), and for setting the concentrations (C_{ED1_+SD}, C_{ED1_M}, C_{ED1_-SD}) determined in this way, respectively, as the estimated target concentration of the associated temporal concentration course, wherein said determining is for a group of temporal concentration courses, in particular models or PK curves (PK50_{+SD}, PK50_{M} and PK50_{-SD}) which are associated with the same dose from a plurality of temporal concentration courses, in particular PK models or PK curves, which are stored in the data storage (M) and are associated with the same dose;
for applying the first application dose (D₁) following the initial dose (D₀) in response to the actuation of the requesting device (1) at the first actuation point of time (tB₁);
for determining, for the temporal concentration courses of the group, at which time after the application of the first application dose (D₁) the concentration in the body, in particular in the blood, will, respectively, have dropped again to the estimated target concentration of the associated temporal concentration course, and for setting, respectively, for the temporal concentration courses of the group, the determined time as the calculated target concentration time (tB_{2_+SD}, tB_{2_M}, tB_{2_-SD});
for detecting or determining the second actuation point of time (tB₂) for requesting a second or subsequent application dose (D₂);
for determining a temporal concentration course from the group as an individual temporal concentration course, in particular as an individual PK curve (PK_{Ind}), while considering the respective difference between the calculated target concentration time (tB_{2_+SD}, tB_{2_M}, tB_{2_-SD}) of each temporal concentration course from the group and the second actuation point of time (tB₂), in particular the temporal concentration course which has the smallest difference between its calculated target concentration time (tB_{2_+SD}, tB_{2_M}, tB_{2_-SD}) and the second actuation point of time (tB₂); and
for considering the determined temporal individual concentration course, in particular the determined individual PK curve (PK_{Ind}), upon predetermining the amount of one or several of the subsequent application doses (D₃, Dₙ, Dₙ₊₁, . . . ).

31. The nasal applicator (100) according to claim 9, wherein the control device (9) is further programmed
for determining the concentration of the substance (S) present in the body or blood of the user at least at one considered actuation point of time (tBₙ₋₁) or at least one considered predetermination point of time (tVₙ₋₁), and for setting this concentration as the target concentration (C_{ED});
and wherein the control device (9) is further programmed
for setting, at the next predetermination point of time (tVₙ), the amount of the next application dose (Dₙ) such that the concentration in the body or in the blood of the user decreases again to the target concentration (C_{ED}) only after a predetermined time period has elapsed, beginning from the dispensing point of time (tAₙ) of the next application dose (Dₙ).

32. The nasal applicator (100) according to claim 1, wherein
the evaluation of the actuation behavior of the user detected by the detection device (11) encompasses detecting actuations of the requesting device (1) by the user at actuation points of time (tB_{1_v1}, tB_{2_v1}, tB_{1_v2}, tB_{2_v2}) within one of the locking periods (T_vain₁, T_vain₂, . . . , T_vainₙ₋₁, T_vainₙ, T_vainₙ₊₁, . . . );
the control device (9) is further programmed for determining a concentration (C(t)) in the body, in particular in the blood, associated with the actuation points of time (tB_{1_v1}, tB_{2_v1}, tB_{1_v2}, tB_{2_v2}) within one of the locking periods (T_vain₁, T_vain₂, . . . , T_vainₙ₋₁, T_vainₙ, T_vainₙ₊₁, . . . ), respectively;
setting the target concentration (C_{ED}) or its minimum value to a value respectively above the associated concentration (C(t)).

33. The nasal applicator (100) according to claim 32,
wherein the detection device (11) is further programmed for detecting at least one actuation of the requesting device (1) by the user at actuation points of time (tB₁, tB₂, . . . , tBₙ₋₁, tBₙ, tBₙ₊₁, . . . ) after the locking period (T_vain₁, T_vain₂, . . . , T_vainₙ₋₁, T_vainₙ, T_vainₙ₊₁, . . . );
wherein the control device (9) is further programmed
for determining a concentration (C(t)) in the body, in particular in the blood, associated with the actuation point of time (tB₁, tB₂, . . . , tBₙ₋₁, tBₙ, tBₙ₊₁, . . . ) after the locking period (T_vain₁, T_vain₂, . . . , T_vainₙ₋₁, T_vainₙ, T_vainₙ₊₁, . . . ), respectively;
for determining a temporal concentration course of the group while considering both at least one, preferably the last, actuation point of time (tB_{1_v1}, tB_{2_v1}, tB_{1_v2}, tB_{2_v2}) within the locking period (T_vain₁, T_vain₂, . . . , T_vainₙ₋₁, T_vainₙ, T_vainₙ₊₁, . . . ) as well as at least one, preferably the first, actuation point of time (tB₁, tB₂, . . . , tBₙ₋₁, tBₙ, tBₙ₊₁, . . . ) after the locking period (T_vain₁, T_vain₂, . . . , T_vainₙ₋₁, T_vainₙ, T_vainₙ₊₁, . . . ); and
for considering the determined temporal individual concentration course, in particular the determined individual PK curve (PK_{Ind}), upon predetermining the amount of one or several of the subsequent application doses (D₃, Dₙ, Dₙ₊₁, . . . ).

34. The nasal applicator (100) according to claim 1, wherein
the evaluation of the actuation behavior of the user detected by the detection device (11) encompasses evaluating the actuations of the requesting device (1) by the user at actuation points of time (tB_{1_v1}, tB_{2_v1}, tB_{1_v2}, tB_{2_v2}) within one of the locking periods (T_vain₁, T_vain₂, . . . , T_vainₙ₋₁, T_vainₙ, T_vainₙ₊₁, . . . );
the control device (9) is further programmed for determining an increase in the time intervals between successive actuation points of time (tB_{1_v1}, tB_{2_v1}, tB_{1_v2}, tB_{2_v2}) within one of the locking periods (T_vain₁, T_vain₂, . . . , T_vainₙ₋₁, T_vainₙ, T_vainₙ₊₁, . . . ) or the absence of further actuation points of time (tB_{1_v1}, tB_{2_v1}, tB_{1_v2}, tB_{2_v2}) within the locking period (T_vain₁, T_vain₂, . . . , T_vainₙ₋₁, T_vainₙ, T_vainₙ₊₁, . . . ), and for defining a point of time starting from which the degree of increase or the absence satisfies predetermined criteria;
the control device (9) is further programmed for determining a concentration (C(t)) in the body, in particular in the blood, associated with the defined set point of time;
setting the target concentration (C_{ED}) or its minimum value to a value above the associated concentration (C(t)), respectively.

35. The nasal applicator (100) according to claim 34,
wherein the control device (9) is further programmed
for evaluating at least one detected actuation of the requesting device (1) by the user at actuation points of time (tB₁, tB₂, . . . , tBₙ₋₁, tBₙ, tBₙ₊₁, . . . ) after the locking period (T_vain₁, T_vain₂, . . . , T_vainₙ₋₁, T_vainₙ, T_vainₙ₊₁, . . . );
wherein the control device (9) is further programmed
for determining a concentration (C(t)) in the body, in particular in the blood, associated with the actuation point of time (tB₁, tB₂, . . . , tBₙ₋₁, tBₙ, tBₙ₊₁, . . . ) after the locking period (T_vain₁, T_vain₂, . . . , T_vainₙ₋₁, T_vainₙ, T_vainₙ₊₁, . . . ), respectively;
for determining a temporal concentration course of the group while considering both a, preferably the last, actuation point of time (tB_{1_v1}, tB_{2_v1}, tB_{1_v2}, tB_{2_v2}) within the locking period (T_vain₁, T_vain₂, . . . , T_vainₙ₋₁, T_vainₙ, T_vainₙ₊₁, . . . ) as well as a, preferably the first, actuation point of time (tB₁, tB₂, . . . , tBₙ₋₁, tBₙ, tBₙ₊₁, . . . ) after the locking period (T_vain₁, T_vain₂, . . . , T_vainₙ₋₁, T_vainₙ, T_vainₙ₊₁, . . . ); and
for considering the determined temporal individual concentration course, in particular the determined individual PK curve (PK_{Ind}), upon predetermining the amount of one or several of the subsequent application doses (D₃, Dₙ, Dₙ₊₁, . . . ).

36. A system comprising
one or more nasal applicators (100) according to any one of the preceding claims;
one or more peripheral devices (101);
wherein the one or the several nasal applicators (100) are, or are prepared or are programmed to be in signal communication or in communication connection with one or with several of the peripheral devices (101).
